(19) United States
(12) Patent Application Publication
(10) Pub. No.: US 2025/0082794 A1
(43) Pub. Date: Mar. 13, 2025

(54) ANTIMONY CHELATES FOR TARGETED AUGER THERAPY AND IMAGING DIAGNOSTICS

(71) Applicants: Wisconsin Alumni Research Foundation, Madison, WI (US); UT-Battelle, LLC, Oak Ridge, TN (US)

(72) Inventors: Jonathan W. Engle, Middleton, WI (US); Aeli Olson, Verona, WI (US); Nikki Thiele, Kingston, TN (US); Briana R. Schrage, Knoxville, TN (US); Md Faizul Islam, Knoxville, TN (US)

(21) Appl. No.: 18/826,309

(22) Filed: Sep. 6, 2024

Related U.S. Application Data
(60) Provisional application No. 63/581,021, filed on Sep. 7, 2023.

(51) Int. Cl. A61K 51/04; A61K 51/06; A61K 51/08; A61K 51/10

(52) U.S. Cl. CPC A61K 51/0497; A61K 51/0478; A61K 51/06; A61K 51/088; A61K 51/1096

(57) ABSTRACT

Antimony (Sb) chelates for use in targeted auger therapy and imaging diagnostics are provided. Also provided are methods of treating a subject in need of treatment using the antimony chelates. The chelates comprise a chelating ligand that binds an Sb radionuclide in a +5-oxidation state, such as ¹¹⁹Sb and ¹¹⁷Sb. The chelating ligand renders the Sb radionuclide stable and inert in vivo and enables the conjugation of the chelate to a biological targeting vector.

### CROSS-REFERENCE TO RELATED APPLICATIONS

The present application claims priority to U.S. provisional patent application No. 63/581,021 that was filed Sep. 7, 2023, the entire contents of which are incorporated herein by reference.

### REFERENCE TO GOVERNMENT RIGHTS

This invention was made with government support under DE-AC05-00OR22725 and DE-SC0023012 awarded by the US Department of Energy. The government has certain rights in the invention.

### BACKGROUND

In pursuit of safer and more effective treatment options for cancer, targeted radionuclide therapy (TRT) has risen to the forefront over the last two decades. TRT leverages the cytotoxic properties of particle-emitting radioactive ions coupled with the specificity of tumor-seeking biomolecules to selectively annihilate diseased cells. To date, many efforts have focused on the application of beta (β⁻) and alpha (α) particles in TRT, leading to the recent regulatory approvals of several ¹⁷⁷Lu-based targeted β radiopharmaceuticals and promising clinical trial results with α emitters such as ²²⁵Ac, ²¹²Pb, and ²¹¹At. However, the potential of a third type of particle radiation, low-energy electrons known as Auger and conversion electrons (ACEs), remains largely untapped.

Auger electrons are ejected, potentially in a cascade, from core atomic orbitals of radioactive ions following electron capture or internal conversion, whereas conversion electrons result directly from the latter decay process. Both types of electrons offer several unique advantages in comparison to their α- and β-particle congeners. Specifically, ACEs are characterized by path lengths typically shorter than the diameter of a single cell, rather than several cells (α) or hundreds to thousands of cells (β). Accordingly, ACE emitters can deposit high linear energy transfer (LET) radiation within an extremely localized area. Additionally, ACE emitters decay directly to stable daughter nuclides. These short decay chains alleviate concerns over the release and redistribution of radioactive daughter ions to distant sites, a problem associated with the use of most a emitters. For these reasons, ACE emitters are potentially unrivaled in their capacity to precisely target and destroy micrometastases and single disseminated cancer cells with minimal damage to surrounding healthy tissue. This level of precision would be highly beneficial in treating cancer without adverse side effects.

Antimony-119 (¹¹⁹Sb, half-life (t_{1/2})=38.19 h, electron capture (EC)=100% (Auger electron (AE)) is considered one of the most promising AE emitting radionuclides for TRT applications and has potential applications in theranostic nuclear medicine with its imaging radioisotope congener antimony-117 (¹¹⁷Sb, t_{1/2}=2.8 h, γ=85.9%, 158.6 keV, EC=97.3%, β+=1.8%).

With emission of 23-24 AEs and low photon emission, ¹¹⁹Sb can provide a clean dose distribution profile. (Eckerman, K. F.; Endo, A. MIRD: Radionuclide Data and Decay Schemes, 2nd ed.; Society of Nuclear Medicine, 2008.) However, an outstanding challenge that hinders ¹¹⁹Sb advancement into clinical studies is the lack of suitable bifunctional chelators for this radiometalloid, which are critical for stably retaining the radionuclide to a targeting vector in vivo. The identification of suitable chelators for Sb is particularly challenging because of its metalloid character, multiple stable oxidation states, and tendency to hydrolyze in aqueous solution.

Of the few efforts reported to date towards developing chelators for targeted Auger therapy with ¹¹⁹Sb, only one chelator, a trithiol ligand, has been demonstrated to form a stable complex with Sb(III) in vitro. (Chen, C. et al., RSC Adv 2022, 12 (10), 5772-5781; Tóth-Molnar, E. et al., Inorg Chem 2021, No. Iii; and Olson, A. P. et al., Inorg Chem 2021, 60 (20), 15223-15232.) No Sb(V) chelator has been reported for radioantimony application. Thus, there remains a critical need for effective Sb-chelating agents for ¹¹⁹Sb in TRT therapy and diagnostic applications.

### SUMMARY

Antimony (Sb) chelates for use in targeted Auger therapy and imaging diagnostics and methods of treating a subject in need of treatment using the antimony chelates are provided.

One embodiment of an antimony chelate includes an antimony (Sb(V)) radionuclide bound to a chelating ligand having the chemical structure:

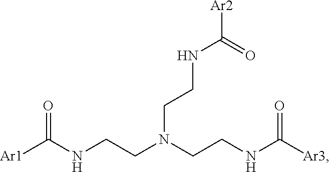

or a pharmaceutically acceptable salt thereof, where Ar1, Ar2, and Ar3 are independently selected from substituted or unsubstituted catechol groups and substituted or unsubstituted hydroxypyridinone (HOPO) groups.

One embodiment of a method of treating a subject in need of treatment includes the step of administering a therapeutically effective amount of an antimony chelate of a type described herein to the subject.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings.

FIG. 1A shows the chemical structures of Sb(V) chelating ligands having catechol groups, 1,2-HOPO groups, or a combination thereof.

FIG. 1B shows the chemical structures of Sb(V) chelating ligands having catechol groups, methyl (Me)-3,2-HOPO groups, 1,2,3-HOPO groups, or a combination thereof.

FIG. 1C shows the chelating ligand TRENCAM binding Sb(V).

FIG. 2 shows a reaction scheme for the bioconjugation of a carboxylic acid functionalized chelating ligand to Vipivotide tetraxetan, a prostate-specific membrane antigen (PSMA)-targeting peptide

FIG. 3. Representative concentration-dependent radio-TLC traces from [^{120m/122/124}Sb]Sb⁵⁺ radiolabeling of TRENCAM, as described in the Example. Reaction conditions: 0.5 M NH₄OAc (pH 4), 80° C., 60 min, 0.57 μCi of [^{120m/122/124}Sb]Sb⁵⁺, 1×10⁻³-1×10⁻⁸ M TRENCAM, Vₜₒₜ=150 μL. Oxalic acid (0.3 M, 10 μL) was added to the reaction and heated at 80° C. for 10 min before spotting on TLC plates.

FIG. 4. Stability of the ^{1XX}Sb complex of TREN-CAM in human serum at 37° C., formed from radiolabeling TREN-CAM (1 mM or 0.1 mM) at 80° C. in NH₄OAc (pH 4), as described in the Examples.

FIG. 5. Radio-HPLC assessment of [^{1XX}Sb]Sb-TREN-CAM stability in human serum showing that the complex remains intact for up to 72 h. Chromatograms from top to bottom: UV chromatogram (254 nm) of TREN-CAM; UV chromatogram (254 nm) of ^{nat}Sb-TREN-CAM complex; radio chromatogram of TREN-CAM radiolabeled with ^{1XX}Sb; radio chromatogram of [^{1XX}Sb]Sb-TREN-CAM after 24 h in human serum; radio chromatogram of [^{1XX}Sb]Sb-TREN-CAM after 72 h in human serum; radio chromatogram of ^{1XX}Sb control.

FIG. 6. Maximum intensity projection μSPECT/CT fused images (top row) and grayscale μSPECT images (bottom row) of [¹¹⁷Sb]Sb-TRENCAM (left four images) and [¹¹⁷Sb]Sb(OH)₆ (right four images) from 20 min frames collected 90- and 240-min post-tail vein injection of the tracers, as described in the Examples.

FIG. 7. Maximum intensity projection μPET/CT fused images (top two images) and grayscale μPET images (bottom two images) of [¹¹⁷Sb]Sb-TRENCAM (left two images) and [¹¹⁷Sb]Sb(OH)₆ (right two images) from 20 min frames collected 150 min post-tail vein injection of the tracers, as described in the Examples.

FIGS. 8A-8C. Comparison of organ biodistributions of [¹¹⁷Sb]Sb-TREN-CAM and [¹¹⁷Sb]Sb(OH)₆⁻ over time following intravenous injection in mice. μSPECT and μPET image-derived temporal in vivo biodistribution of [¹¹⁷Sb]Sb(OH)₆⁻ (FIG. 8A) and [¹¹⁷Sb]Sb-TREN-CAM (FIG. 8B). Ex vivo biodistribution of both radiotracers (FIG. 8C).

FIG. 9. Preliminary screening of the chelators DOTA, DFO, and TREN-CAM by radio-HPLC. Radiolabeling reactions were carried out at pH 4 (0.5 M NH₄OAc) and 80° C. for 60 min using 21 kBq (0.57 μCi) of ^{1XX}Sb and 1×10⁻³ M of chelator. The final volume of each sample was 150 μL. The control sample (^{1XX}Sb only) received water instead of chelator. After 60 min, the reactions were analyzed by radio-HPLC.

FIGS. 10A-10B. Radiolabeling of TREN-CAM with ^{1XX}Sb. (FIG. 10A) Radiochemical yields as a function of time and temperature. Each data point represents an individual sample. Reaction conditions: 21 kBq ^{1XX}Sb, 10⁻³ M chelator, pH 4 NH₄OAc (0.5 M), V_{T}=150 μL. (FIG. 10B) Radiochemical yields as a function of ligand concentration and solution pH (n=3 per data point). Reaction conditions: 21 kBq ^{1XX}Sb, 0.5 M NH₄OAc (pH 4, 6) or 0.01 M HCl (pH˜2), V_{T}=150 μL, 60 min, 80° C. All samples received oxalic acid (3 μmol) at the appropriate timepoint, after which they were heated for 10 min at 80° C. before being spotted on TLC plates.

FIGS. 11A-11D. Structural characterization of the ^{nat}Sb-TREN-CAM complex in aqueous solution by X-ray spectroscopy (XAS) and density functional theory (DFT) calculations. (FIG. 11A) Normalized Sb K-edge XANES spectra from aqueous solutions of [Sb(TREN-CAM)]⁻ and KSb(OH)₆ at pH 9 versus solid Sb₂O₅ and Sb₂O₃ references. (FIG. 11B) K-edge EXAFS spectrum of [Sb(TREN-CAM)]⁻ at pH 9. (FIG. 11C) Magnitude of the Fourier transform EXAFS (black circles) and the real component of the FT (empty circles). The fit represents model scattering paths associated with the [Sb(TREN-CAM)]⁻ structure. Spectra are not phase-shift corrected. (FIG. 11D) DFT-optimized structure of [Sb(TREN-CAM)]⁻ with the representative Sb—O bonding NBO, which is predominantly localized on the oxygen atom and includes about 17% Sb character. The 0.06 amplitude cut-off was used for the orbital visualization. Hydrogen atoms on the carbon skeleton are omitted for clarity.

FIG. 12. HRMS of TREN-CAM-PEG₁-PSMA.

FIG. 13. HRMS of TREN-CAM-PEG₁-DUPA.

FIG. 14. HRMS of TREN-CAM-PEG₃-PSMA.

### DETAILED DESCRIPTION

Antimony (Sb) chelates for use in targeted Auger therapy and imaging diagnostics are provided. Also provided are methods of treating a subject in need of treatment using the antimony chelates. The chelates comprise a chelating ligand that binds an Sb radionuclide in a +5-oxidation state (Sb(V)), such as ¹¹⁹Sb and ¹¹⁷Sb. The chelating ligand renders the Sb radionuclide stable and inert in vivo and enables the conjugation of the chelate to a biological targeting vector.

### Sb Chelates.

The Sb chelates are compounds that include an Sb radionuclide bound by a chelating ligand having the chemical structure:

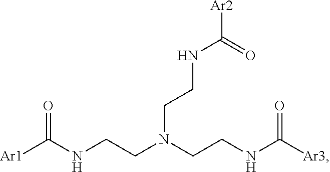

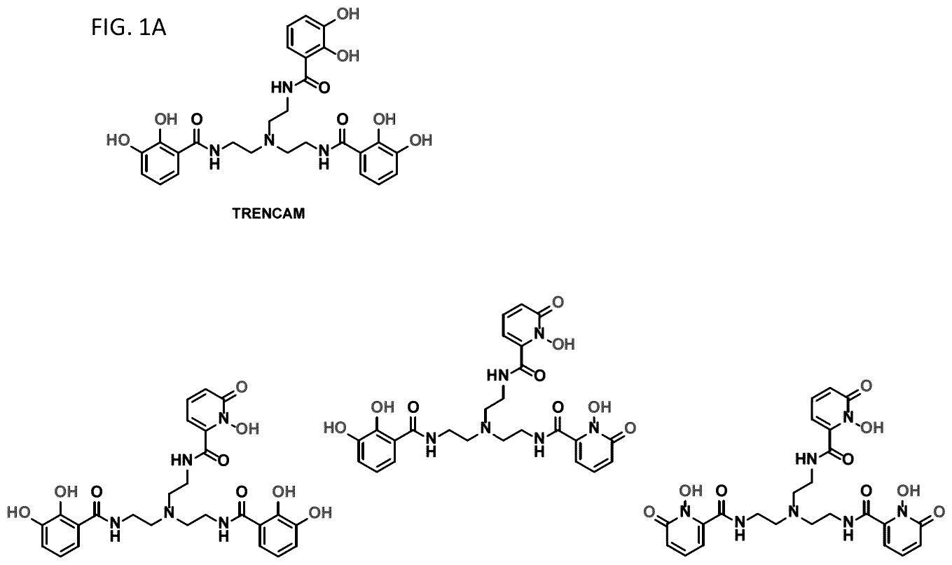

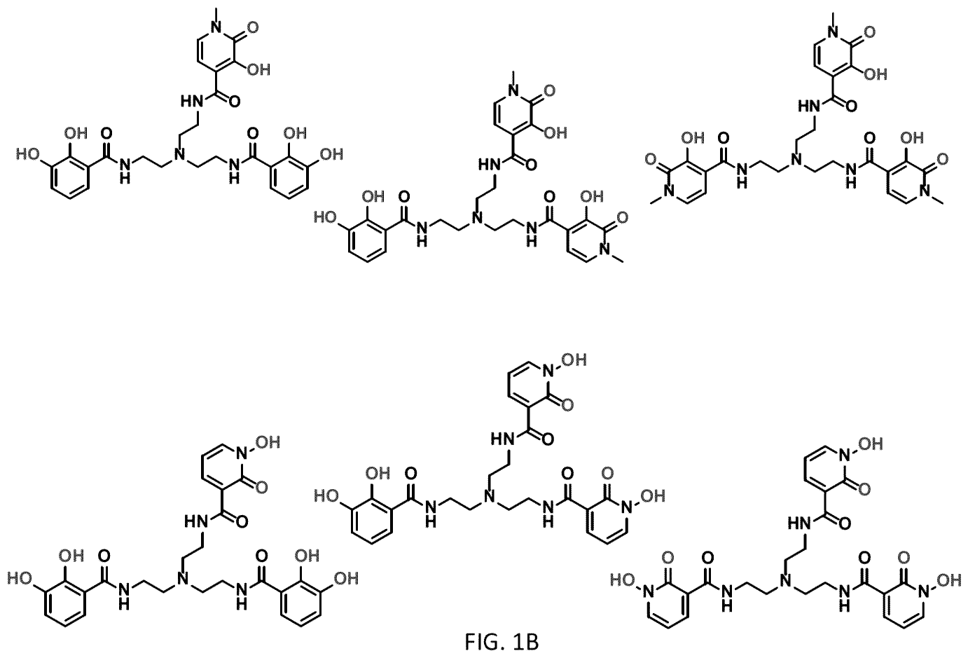

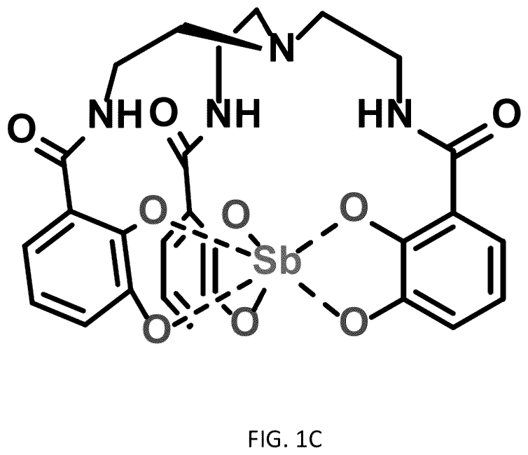

or a pharmaceutically acceptable salt thereof, where Ar1, Ar2, and Ar3 are independently selected from substituted or unsubstituted catechol groups and substituted or unsubstituted hydroxypyridinone (HOPO) groups. Examples of HOPO groups include 1,2-HOPO groups, methyl (Me)-3,2-HOPO groups, and 1,2,3-HOPO groups. For purposes of illustration, FIGS. 1A and 1B show embodiments of chelating ligands having three catechol groups (“TRENCAM”) (FIG. 1A, upper panel), having a combination of catechol and 1,2-HOPO groups or three 1,2-HOPO groups (FIG. 1A, lower panels), having a combination of catechol and Me-3,2-HOPO groups or three Me-3,2-HOPO groups (FIG. 1B, upper panels), and having a combination of catechol and 1,2,3-HOPO groups or three 1,2,3-HOPO groups (FIG. 1B, lower panels). As illustrated in FIG. 1C, oxygen substituents on the phenyl rings of the catechol groups or on the pyridyl rings of the HOPO groups act as Sb coordinating groups in the Sb chelates. It should be noted that, while each of the hydroxyl groups in the Sb chelate shown in FIG. 1C is deprotonated, all or some of the hydroxyl groups of the ligands in the Sb chelates described herein may be in their protonated form.

Pharmaceutically acceptable salts of the Sb chelate compounds described herein include salts which retain the desired therapeutic and/or diagnostic abilities of the Sb chelate compounds.

While the Sb chelates have useful applications in dosimetry and other studies even without an attached biological targeting vector, for use in TRT and targeted diagnostic applications it is desirable for the chelating ligand to be conjugated to a biological targeting vector to facilitate the delivery of the Sb radionuclide to a desired location in the subject being treated. Therefore, the Sb chelates are desirably bifunctional chelates that include one or more reactive ring substituents on their catechol and/or HOPO groups and/or on the backbone of the tris(2-aminoethyl)amine (TREN) scaffold. These reactive functional groups can form a covalent linkage to a biological targeting vector, either through the formation of a direct covalent bond to the biological targeting vector or through the formation of a covalent bond to a molecular linker attached to the biological targeting molecule.

Optionally, the chelating ligands may include one or more electron withdrawing ring substituents, electron donating ring substituents, and/or solubilizing ring substituents on the phenyl ring of a catechol group and/or on a pyridyl ring of a HOPO group. The electron withdrawing groups are ring substituents that decrease the electron density on the ring, relative to the electron density on the ring in the absence of a substituent at that ring position. Examples of electron withdrawing groups include NO₂ groups, halo groups, sulfonyl groups, cyano groups, trihalomethyl groups, and trifluoromethylsulfonyl groups. The electron donating groups are ring substituents and increase the electron density on the ring, relative to the electron density on the ring in the absence of a substituent at that ring position. Examples of electron donating groups include alkoxy groups, such as methoxy groups, hydroxy groups, amino groups, phenyl groups, alkyl groups, acyloxy groups, and oxido groups. The solubilizing groups are ring substituents that increase the solubility of the chelating ligands in water, relative to the water solubility of the chelating agent in the absence of a substituent at that ring position. Examples of solubilizing groups include sulfonic acid groups (—SO₃H), polyethylene glycol (PEG) groups, alcohol groups, amine groups, and carboxylic acid groups.

The catechol groups (i.e., Ar1, Ar2, and/or Ar3) of the chelating ligands can be represented by the structure:

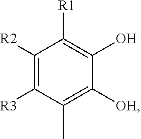

where R1, R2, and R3 are independently selected from H atoms, ring substituents comprising a functional group capable of forming a covalent bond to a biological targeting vector, ring substituents comprising a biological targeting vector, electron withdrawing ring substituents, electron donating ring substituents, and solubilizing ring substituents, including those described herein.

The 1,2-HOPO groups (i.e., Ar1, Ar2, and/or Ar3) of the chelating ligands can be represented by the structure:

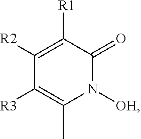

where R1, R2, and R3 are independently selected from H atoms, ring substituents comprising a functional group capable of forming a covalent bond to a biological targeting vector, ring substituents comprising a biological targeting vector, electron withdrawing ring substituents, electron donating ring substituents, and solubilizing ring substituents, including those described herein.

The Me-3,2-HOPO groups (i.e., Ar1, Ar2, and/or Ar3) of the chelating ligands can be represented by the structure:

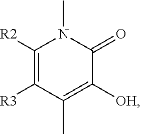

where R2 and R3 are independently selected from H atoms, ring substituents comprising a functional group capable of forming a covalent bond to a biological targeting vector, ring substituents comprising a biological targeting vector, electron withdrawing ring substituents, electron donating ring substituents, and solubilizing ring substituents, including those described herein.

The 1,2,3-HOPO groups (i.e., Ar1, Ar2, and/or Ar3) of the chelating ligands can be represented by the structure:

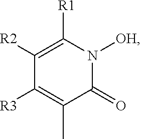

where R1, R2, and R3 are independently selected from H atoms, ring substituents comprising a functional group capable of forming a covalent bond to a biological targeting vector, ring substituents comprising a biological targeting vector, electron withdrawing ring substituents, electron donating ring substituents, and solubilizing ring substituents, including those described herein.

### Sb Chelate Synthesis.

The Sb radionuclides can be produced using known methods, such as charged particle bombardment of tin or the production of ¹¹⁹Te followed by development of ¹¹⁹Te/¹¹⁹Sb generator. Once formed, the Sb radionuclides can be purified via radiochemical separation by solvent extraction, metal complexation followed by solvent extraction, precipitation, or ion-exchange. (See, for example, Randhawa, Parmissa, et al. Current radiopharmaceuticals 14.4 (2021): 394.)

The chelating ligands can also be synthesized using known procedures. (See, for example, Joaqui-Joaqui, et al., Inorg Chem 2020, 59 (17), 12025-12038; Xu, Jide, et al. Inorganic chemistry 43.18 (2004): 5492-5494; Xu, Jide, et al. Journal of the American Chemical Society 117.27 (1995): 7245-7246; and Nelson, Joshua J M, et al. Communications Chemistry 3.1 (2020): 7.)

The Sb radionuclides can be combined in solution with the chelating ligands to form the Sb chelates, as illustrated in the Examples.

### Bifunctional Chelating Ligands.

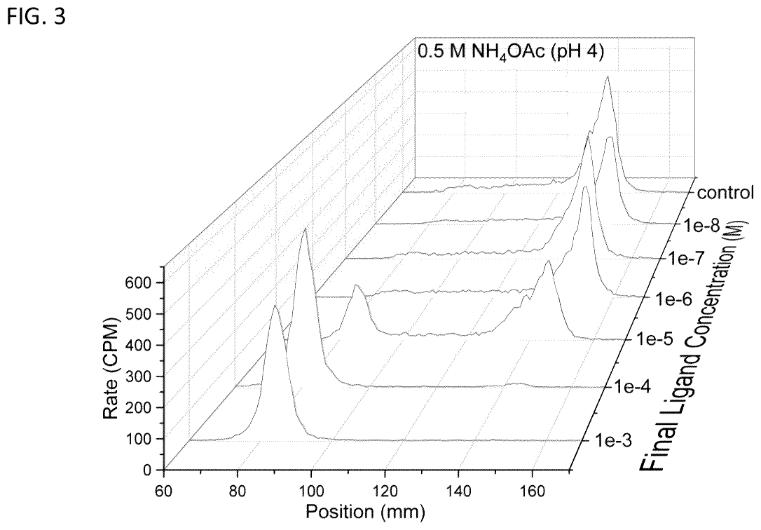

Examples of reactive groups with which the catechol groups or the HOPO groups of a chelating ligand can be functionalized to form bifunctional chelating ligands include amine (—NH₂) and carboxylic acid substituents (—C(O)OH). An illustrative example of a chelating ligand having a carboxylic acid substituent is shown in FIG. 3 (inside dashed box). Other reactive functional groups for binding biological target vectors include, but are not limited to, —NCS, acyl chlorides, —NCO, alkynes, azides, tetrazines, maleimides, and activated esters, such as tetrafluorophenyl and N-hydroxysuccinimide. The reactive groups may themselves be bound to a catechol or HOPO group via an organic linker chain. In some examples of the bifunctional chelates, a terminal amine group or a terminal thiocyanate group is bonded to a catechol group or a HOPO group via an organic linker chain that includes, or consists of only, an ether chain (—R—O—R—, where each R represents an alkyl chain of two or more (e.g., from 2 to 10) carbon atoms), wherein the organic linker chain is covalently bonded to the aromatic ring of the catechol or HOPO group via an amide bond (—C(O)—NH—). The ether chain may be a polyether polyol chain (—[R—O]ₙ—), where n represents the number of repeat units; n may be, for example, in the range from 2 to 10). Examples of polyether polyol chains include polyethylene glycol chains.

Methods of synthesizing bifunctional chelates having substituents with terminal amine groups are illustrated in Example 2. In these methods, an alkylene glycol diamine having one tert-butyloxycarbonyl (Boc)-protected amine group reacts with the carboxyl group of 2,3-bis(benzyloxy)-4-((benzyloxy)carbonyl)benzoic acid to form a benzyl 2,3-bis(benzyloxy)-4-((2-(2-((tert-butoxycarbonyl)amino)alkoxy)alkyl)carbamoyl)benzoate. The benzyl 2,3-bis(benzyloxy)-4-((2-(2-((tert-butoxycarbonyl)amino)alkoxy)alkyl)carbamoyl)benzoate can then be converted into its corresponding benzoic acid. The carbonyl group of said benzoic acid is then reacted with the amine group of N,N′-(((2-aminoethyl)azanediyl)bis(ethane-2,1-diyl))bis(2,3-bis(benzyloxy) benzamide), followed by the conversion of the benzyloxy (OBn) groups into hydroxyl groups (—OH) to provide a chelating ligand with a reactive terminal amine group.

The alkylene glycol diamines, may be polyalkylene glycol diamines, such as polyethylene glycol diamines having the structure (shown below in an unprotected form):

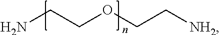

where n represents the number of repeat units. The value of n ranges from 1 to 10 in some embodiments of the methods. However, higher n values are also suitable. It should be noted that polyethylene glycol diamines are only illustrative examples of polyalkylene glycol diamines.

Once the reactive amine substituents have been installed on the chelating ligands, they can be converted into other reactive groups, including other reactive groups described herein, using known chemistries. For example, amine terminated substituents can be converted into terminal isothiocyanate groups by reacting the amine group with an aliphatic or aryl diisothiocyanate, such as p-phenylene diisothiocyanate.

### Chelating Ligands Conjugated to a Biological Targeting Vector (“Targeted Conjugates”).

The biological targeting vectors are vectors that specifically bind to a particular type of cell, such as a cancer cell, and direct the chelates to a region of the body to be treated and/or imaged using the Sb radionuclides. For example, in ¹¹⁹Sb-based TRT or ¹¹⁷Sb-based diagnostics, the biological targeting vectors may have a specific affinity to a cellular disease marker, such as a receptor protein overexpressed in cancer, to deliver the ^{119/117}Sb chelate to the location of the cancer, where it emits short range, cytotoxic Auger electrons to diseased cells or emits a detectable gamma-ray or positron signal.

Biological targeting vectors include hormones, signaling molecules, binding moieties, antibodies, antibody fragments (e.g., an antigen-binding fragment), binding proteins, binding peptides, binding polypeptides (such as a selective targeting oligopeptide containing up to 50 amino acids), binding proteins, enzymes, nucleobase-containing moieties (such as oligonucleotide, DNA or RNA vectors, or aptamers), or lectins.

Illustrative examples of targeting vector antibodies include belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, Etaracizumab, and fragments thereof.

Illustrative examples of targeting vector peptides include a prostate specific membrane antigen (“PSMA”) binding peptide, a somatostatin receptor agonist, a bombesin receptor agonist, and a seprase (fibroblast activation protein) binding compound.

Illustrative antigens that can be targeted include CD20, CD147, CD22, CD37, HLA-DR, CD38, HK2, PSMA, CD46, CD33, CD45, CEA, PD-L1, A33, HER2, Ep-CAM, TAG-72, CSA-p, HK2, EGFR, PSCA, CEA/HSG, CEA/hapten, SSTR, GRPR, Integrins, GRPR/PSMA, Integrin/GRPR, PSMA/GRPR, GnRH-R, NK-1R, VEGF/Integrins, MMP-1, CCK2R, CXCR4, Neurotensin, Upar, MC1R, and GLP-1R, description of which can be found in Zhang, Taotao, et al. “Carrier systems of radiopharmaceuticals and the application in cancer therapy.” Cell Death Discovery 10.1 (2024): 16.

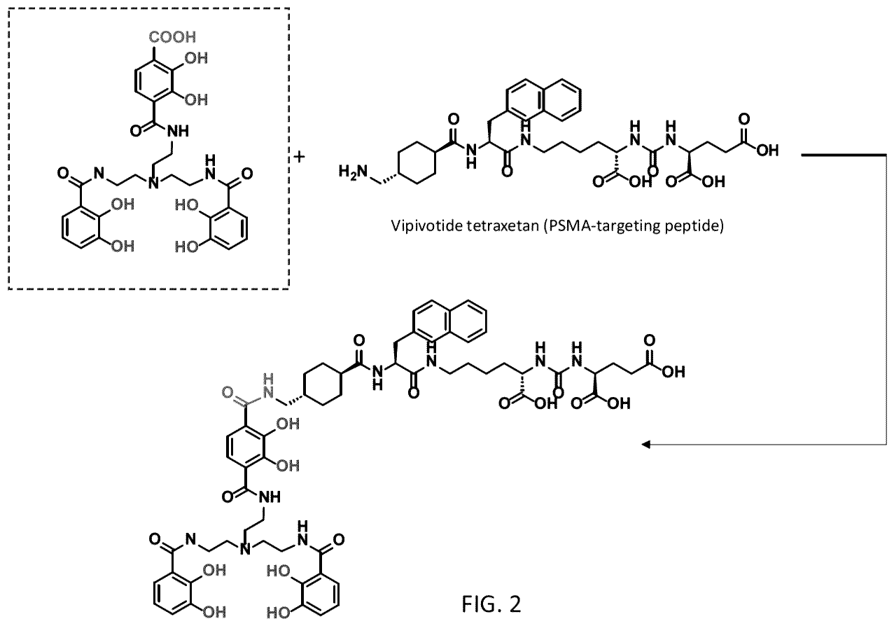

Chemistries for conjugating biological targeting vectors to the chelating ligands using reactive functional groups are known. (See, for example, Price, Eric W. et al., Chemical Society Reviews 43.1 (2014): 260-290.) Suitable bioconjugation reactions include: peptide coupling reactions between a carboxylic acid group and a primary amine with a coupling reagent; peptide coupling reactions between activated esters of tetrafluorophenyl or N-hydroxsuccinimide and a primary amine; thiourea bond formation between an isothiocyanate and a primary amine; thioether bond formation between a maleimide and a thiol; Cu(I) catalyzed Huisgen 1,3-dipolar cycloaddition between an azide and an alkyne; and strain-promoted Diels-Alder reaction between a tetrazine and transcyclooctene. By way of illustration, FIG. 2 shows a reaction scheme for the bioconjugation of a carboxylic acid functionalized chelating ligand to Vipivotide tetraxetan, a prostate-specific membrane antigen (PSMA)-targeting peptide.

Targeted conjugates can be synthesized by reacting the reactive substituent on a bifunctional chelating ligand with a reactive group on a biological targeting vector to form a covalent bond. The biological targeting vectors may inherently have groups, such as carboxyl groups, amine groups, thiol groups, or isothiocyanate groups, that react with reactive groups on the substituents of the bifunctional chelating ligands to form covalent bonds. However, if the biological targeting vectors lack such groups, they can be functionalized with such groups.

By way of illustration, reacting an amine group of a bifunctional chelating ligand with a carboxyl group on biological targeting vector (or vice versa) can form an amide bond. Alternatively, reacting an isothiocyanate group of a bifunctional chelating ligand with an amine group on biological targeting vector (or vice versa) can form a thiourea bond (—HN—C(S)—NH—). In other embodiments, a bifunctional chelating ligand with a maleimide group or an oxadiazolyl methyl sulfone group can react with a thiol group on biological targeting vector (or vice versa) can form a covalent bond.

Examples of biological targeting vectors to which the bifunctional chelating ligands can be conjugated include vipivotide tetraxetan and 2-[3-(1,3-dicarboxypropyl)ureido]pentanedioic acid (DUPA). DUPA is a glutamate urea that is used as a targeting vector to selectively deliver cytotoxic agents to prostate cancer cells.

Illustrative reaction schemes that can be used to conjugate bifunctional chelating ligands to biological vectors are provided in Example 2. DUPA and vipivotide tetraxetan are used as representative targeting vectors in Example 2. Other targeting vectors functionalized with these groups can also be used in the reactions.

### TRT and Diagnostics.

The ¹¹⁹Sb chelates can be used to treat a subject, such as a human, in need of Auger electron radionuclide therapy and the ¹¹⁷Sb chelates can be used to treat a subject in need of diagnostic imaging by administering a therapeutically effective amount of the ¹¹⁷Sb chelate or the ¹¹⁹Sb chelate to the subject. The ¹¹⁹Sb and ¹¹⁷Sb chelates may be administered as a therapeutic and diagnostic (“theranostic”) radionuclide pair. The subject being treated or imaged may be, for example, a mammal, such as a human, a veterinary animal (e.g., a pet), an animal kept as livestock, or a research animal.

### Auger Electron Radionuclide Therapy.

In Auger electron radionuclide therapy, ¹¹⁹Sb decays via electron capture to emit Auger electrons. These Auger electrons are well suited for radiotherapy application due to their low energies and short path lengths, which result in a high linear energy transfer (LET) and thus a highly selective therapeutic radiation dose at the cellular level. ¹¹⁹Sb is particularly suited for use as an Auger electron-emitting radionuclide due to its high Auger electron yields, a half-life that is compatible with the biological half-lives of common delivery systems used in radiopharmaceuticals, and the absence of the emission of dosimetrically problematic gamma-rays. The Auger electrons emitted by the ¹¹⁹Sb chelates are useful in cancer therapy because these electrons are highly cytotoxic when deposited close to cellular DNA, while their short path-length and high LET produce minimal toxicity to surrounding healthy tissue.

For the purposes of this disclosure, a therapeutically effective amount of a ¹¹⁹Sb chelate refers to an amount that is effective to prevent, inhibit, treat, and/or study a particular disease, such as a cancer, or to alleviate a symptom thereof. A therapeutically effective amount may vary depending on the size, age, and/or gender of the subject, and/or a desired therapeutic outcome. Guidance for determining the dosage for a therapeutically effective amount of ¹¹⁹Sb can be obtained from, for example, dosimetric calculations that assess the targeted radiotherapeutic capacity of ¹¹⁹Sb. (See, for example: Thisgaard H; Jensen M Med. Rhys, 2008, 35(9), 3839-3846; Hsiao Y Y et al., Int. J. Radiat. Biol, 2014, 90(5), 392-400; and Falzone N et al., J. Nucl. Med, 2015, 56(9), 1441-1446.)

Subjects in need of Auger electron radionuclide therapy include living beings (e.g., humans) that have been diagnosed with, or that are suspected of having, a disease characterized by the proliferation of unwanted cells, including various types of cancer. Such cancers include, for example, breast cancer, colorectal cancer, cervical cancer, ovarian cancer, liver cancer, bladder cancer, hepatoma, hepatocellular carcinoma, brain cancer, lung cancer, gastric or stomach cancer, pancreatic cancer, thyroid cancer, kidney or renal cancer, prostate cancer, melanoma, sarcomas, and carcinomas.

Possible therapeutic outcomes of the Auger electron radionuclide therapy include decreasing the number of cancer cells, increasing the life-expectancy of the subject, inhibiting cancer cell proliferation or tumor growth, prevent or reducing metastases, reducing pain associated with cancer, and/or reducing the re-occurrence of cancer following the therapy.

### Imaging Diagnostics.

The ¹¹⁷Sb chelates can be used in in vivo diagnostic medical imaging applications, including single-photon emission computed tomography (SPECT) or positron emission tomography (PET). In the methods of diagnostic imaging a therapeutically effective amount of ¹¹⁷Sb chelate is administered to a subject. A gamma ray signal (SPECT) or a positron signal (PET) is then generated by the ¹¹⁷Sb chelate in vivo. This signal is detected and converted into an image. When an ¹¹⁷Sb chelate is used with a therapeutic radionuclide, such as a ¹¹⁹Sb chelate, as part of a theranostic pair, the ¹¹⁷Sb chelate may be administered before and/or after the administration of the therapeutic radionuclide.

The ¹¹⁷Sb chelates are suited for SPECT imaging based on their gamma-ray emissions (e.g., 158.6 keV gamma rays) and are also suitable for PET imaging based on their emission of positrons with a branching ratio of 1.8%.

A therapeutically effective amount of a ¹¹⁷Sb chelate refers to an amount that is effective to generate a gamma-ray signal or a positron signal of sufficient intensity to generate a SPECT image or a PET image of sufficient quality for its intended purpose. A therapeutically effective amount may vary depending on the size, age, and/or gender of the subject, and/or the required signal intensity. Guidance for determining the dosage for a therapeutically effective amount of ¹¹⁷Sb is provided in the Examples.

Subjects in need of imaging include subjects in need of imaging to: aid in a disease diagnosis, such as a cancer diagnosis; aid in the staging of a disease; measure target expression; enable dosiometry planning and estimation for the subsequent administration of a therapeutic radionuclide; locate a position within the subject for a therapeutic treatment; assess the efficacy of a therapeutic treatment on a disease; assess the functioning of a body part; and/or to assess a change in the severity of a disease, such as cancer. Thus, subjects in need of imaging also include living beings (e.g., humans) that have been diagnosed with, or that are suspected of having, a disease characterized by the proliferation of unwanted cells, including various types of cancer.

### Administration.

The chelates may be administered to a subject by direct injection of the chelate into the bloodstream or a tumor. However, other forms of administration, including, intra-muscular, oral, subcutaneous, intrarectal, inhalation, or topical may be used. For administration via injection the Sb chelates may be formulated as an aqueous solution or suspension. For other means of administration, such as oral administration, the Sb chelates may be formulated a solid powder, a tablet, a pill, a capsule, a gel, or a syrup.

In addition to the Sb chelates, the formulations may include pharmaceutically acceptable carriers, where pharmaceutically acceptable indicates approval by a regulatory agency of the Federal or a state government or listed in the U.S. Pharmacopeia or other generally recognized pharmacopeia for use in animals. For purposes of illustration, pharmaceutically acceptable carriers include diluents, absorption delaying agents, antioxidants, binders, buffering agents, bulking agents or fillers, coatings, dispersants, emulsifiers, isotonic agents, lubricants, preservatives, binders, salts, solvents stabilizers, and surfactants.

### EXAMPLES

### Example 1

This Example demonstrates stable complexation of no-carrier-added, high specific activity radioantimony by the tris-catechol chelator TRENCAM (Scheme 1) and in vivo complex stability assessment through SPECT and PET imaging. To the inventors' knowledge, this is the first reported study of in vivo biological stability and distribution assessment of a radioantimony complex via ¹¹⁷Sb imaging.

Scheme 1: Structures of Chelators Used in this Report.

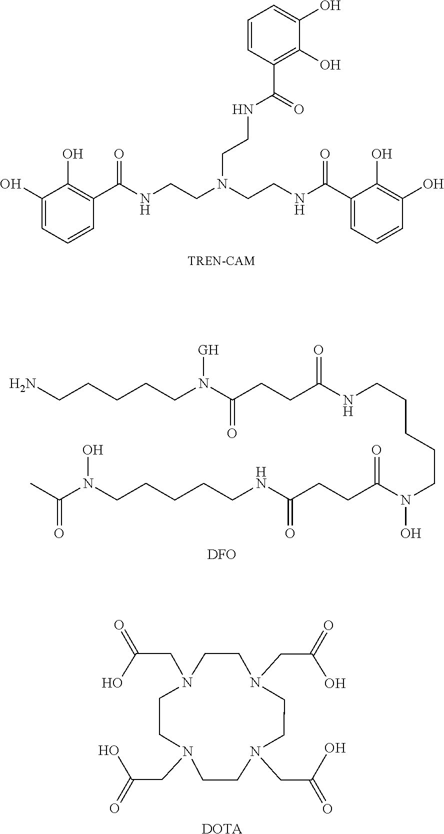

### Methods

TABLE 1
Radioantimony isotopes produced via proton and deuteron bombardment
of natural tin with notable emissions and relevant half-lives

| | Half- | | |
|---|---|---|---|
| | live | | |
| Radioantimony | (h) | Decay | Emission (I_{B}) |
| ¹¹⁷Sb | 2.8 | EC and β⁺ = 100% | γ = 158.56 keV (0.859) |
| | | | β⁺ = 262.4 keV (0.0181) |
| ^{118m}Sb | 5.0 | EC and β⁺ = 100% | γ = 253.6 keV (0.996) |
| | | | β⁺ = 146.8 keV (0.0016) |
| ¹¹⁹Sb | 38.19 | IC = 100% | γ = 23.87 keV (0.165) |
| ^{120m}Sb | 138.24 | EC = 100% | γ = 197.3 keV (0.87) |
| | | | 1171.7 keV (1.0) |
| ¹²²Sb | 65.37 | β⁻ = 97.59% | β⁻ = 523.6 keV (0.667) |
| | | EC and β⁺ = 2.41% | γ = 564.24 keV (0.707) |
| | | | β⁺ = 290.0 (0.00006) |
| ¹²⁴Sb | 1444.8 | β⁻ = 100% | β⁻ = 193.8 keV (0.512) |
| | | | γ = 602.7 keV (0.978) |

Data in Table 1 is from: Symochko, D. M.; Browne, E.; Tuli, J. K. Nuclear Data Sheets for A=119. Nuclear Data Sheets 2009, 110 (11), 2945-3105; Blachot, J. Nuclear Data Sheets for A=117. Nuclear Data Sheets 2002, 95 (3), 679-836; Kitao, K. Nuclear Data Sheets Update for A=118. Nuclear Data Sheets. 1995, pp 99-198; Kitao, K.; Tendow, Y.; Hashizume, A. Nuclear Data Sheets for A=120. Nuclear Data Sheets 2002, 96 (2), 241-390; Tamura, T. Nuclear Data Sheets for A=122. Nuclear Data Sheets 2007, 108 (3), 455-632; and Katakura, J.; Wu, Z. D. Nuclear Data Sheets for A=124. Nuclear Data Sheets 2008, 109 (7), 1655-1877.

### Chelator Screening:

The small ionic radius (6-coordinate=0.60 Å) and high charge of Sb(V) led to the hypothesis that hexadentate or octadentate chelators containing harder oxygen and nitrogen donors may effectively satisfy the coordination preferences of this ion. Based on these criteria, DFO, TREN-CAM, and DOTA were selected for preliminary binding studies.

### Radiolabeling:

### Radiolabeling Optimization

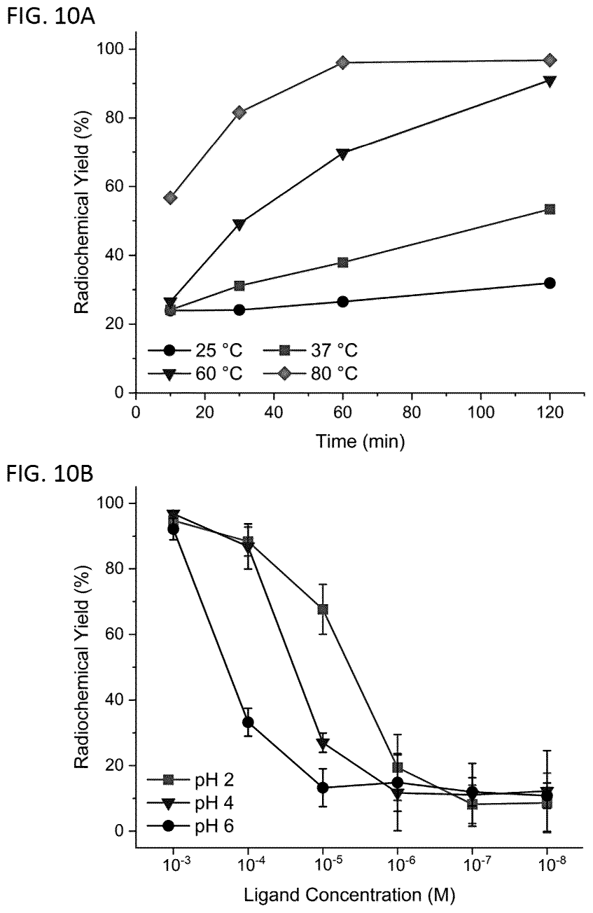

Next, optimal conditions were sought for radiolabeling TREN-CAM with ^{1XX}Sb. Samples were first prepared at a single chelator concentration (1 mM) and pH (4) and spiked with 21 kBq (0.57 μCi) ^{1XX}Sb. After incubating the samples for 10, 30, 60, or 120 min at temperatures of 25, 37, 60, and 80° C., they were analyzed by radio-thin layer chromatography (radio-TLC). Specifically, samples were aliquoted onto reverse-phase C₁₈ aluminum-backed plates and developed with a mobile phase of 0.25 M oxalic acid. Under these conditions, [^{1XX}Sb]Sb-TREN-CAM remained at the baseline (R_{f}=0), whereas “free” ^{1XX}Sb migrated with the solvent front (R_{f}=1). Analysis of control samples, however, revealed that ˜10% of the activity remained bound to the origin in the absence of chelator, which would confound the accurate determination of radiochemical yields (RCYs). Adding a small amount of oxalic acid directly to the reactions and heating them at 80° C. for 10 min was sufficient to ensure migration of all unchelated ^{1XX}Sb in solution, presumably via oxalate complex formation. The radiolabeling results using this method are shown in FIG. 10A. RCYs increased as a function of time, albeit only slightly for complexation reactions conducted at 25° C. Similarly, higher RCYs were achieved at higher reaction temperatures, with complete radiolabeling reached after 60 min at 80° C. or 120 min at 60° C.

As the pH of the reaction solution was increased from 2 to 4 to 6, RCYs declined (FIG. 10B). This trend likely arose from increasing hydroxide competition at higher pH, which will favor the formation of unchelated Sb(V) as Sb(OH)₆⁻. Nevertheless, TREN-CAM was able to quantitatively complex ^{1XX}Sb at a concentration of 10⁻³ M across all pH values studied. Furthermore, nearly complete complexation was observed using 10⁻⁴ M chelator concentration at both pH 2 and 4. Overall, these studies confirm the ability of TREN-CAM to effectively bind ^{1XX}Sb(V) in aqueous solution.

### Solution-State Characterization: NMR and XAS Studies

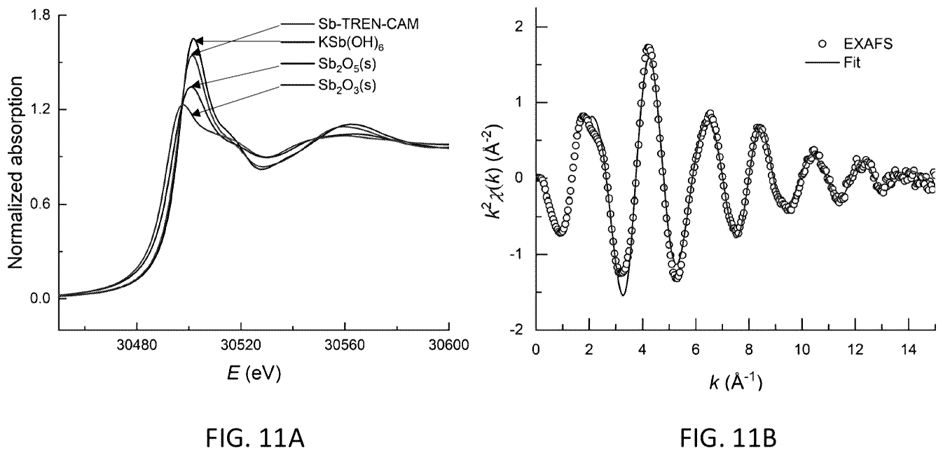

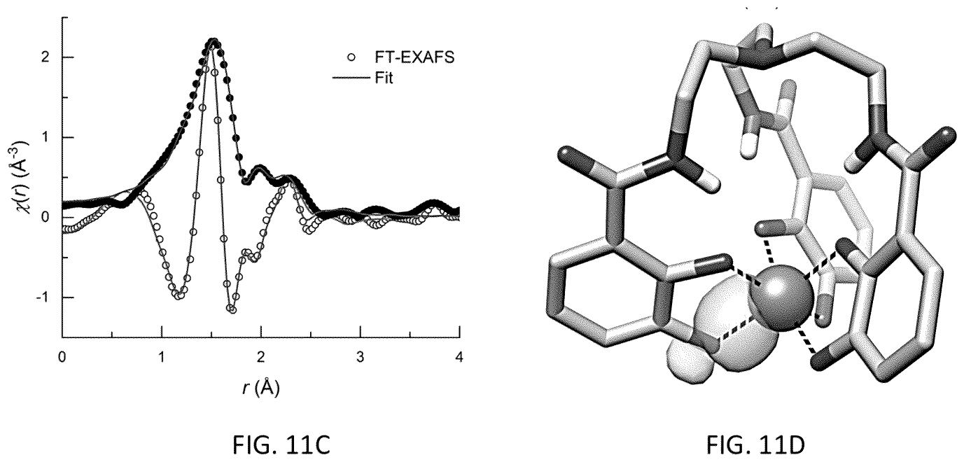

To garner insight into the coordination environment around the Sb center of the TREN-CAM complex in aqueous solution, synchrotron X-ray absorption spectroscopy (XAS) studies were carried out. The sample was prepared in water using KSb(OH)₆ (1 mM) and excess TREN-CAM (14 mM), and the solution pH was adjusted to 9 using NH₄OH. K-edge XAS spectra were acquired at room temperature in fluorescence mode at beamline 8-ID of the National Synchrotron Light Source II. To characterize the oxidation state of [^{nat}Sb(TREN-CAM)]⁻, its X-ray absorption near-edge structure (XANES) spectrum was acquired and compared with those of KSb(OH)₆ (pH 9), Sb₂O₅(s), and Sb₂O₃(s) (FIG. 11A). The absorption edge of [^{nat}Sb(TREN-CAM)]⁻ (30,501 eV) was consistent with the Sb₂O₅ reference and was shifted to a higher energy relative to the Sb₂O₃ reference. These results confirm that Sb was in the +5 oxidation state and did not undergo reduction upon TREN-CAM complexation in aqueous solution. Additionally, the XANES spectrum of [^{nat}Sb(TREN-CAM)]⁻ showed intensities of the white line and post-edge features similar to those of aqueous KSb(OH)₆ in the absence of the ligand, indicating a comparable octahedral environment surrounding Sb(V).

Next, the local coordination of Sb(V) was investigated by examining the extended X-ray absorption fine structure (EXAFS) spectrum of the ^{nat}Sb-TREN-CAM complex. A Fourier transform was applied to the EXAFS data in FIG. 11B, generating real-space functions that provided a tangible depiction of the atomic arrangement around the Sb(V) ion. The Fourier transform EXAFS (FIG. 11C) revealed two intense features at 1.5 Å and 2.3 Å (uncorrected for phase shift), likely originating from the inner-shell Sb—O and nearest-neighbor Sb—C scattering correlations. This interpretation is further supported by good fits of the EXAFS (FIGS. 11B, 11C), which yielded an Sb—O coordination number of 6.7(4) with an average Sb—O bond distance of 1.992(5) Å and an Sb—C coordination number of 4(2) with an average Sb—C distance of 2.79(2) Å. For comparison, EXAFS data of a sample containing catechol (300 mM) and KSb(OH)₆ (7 mM) were also acquired at pH 9 and found to be similar to the data obtained with [Sb(TREN-CAM)]⁻. Notably, for both [Sb(TREN-CAM)]⁻ and [Sb(catechol)₃]⁻, a distinct second shell feature associated with Sb—C scattering paths was observed at approximately 2.3 Å in non-phase corrected real-space FT-EXAFS, confirming complexation of Sb(V) by these organic ligands. By contrast, sharp second-shell features were absent for KSb(OH)₆, reflecting its inorganic coordination environment. Taken together, these results are consistent with hexadentate Sb(V) complexation by the catecholate donors of the TREN-CAM ligand.

### Density Functional Theory Calculations

The binding interactions between TREN-CAM and Sb(V) were further explored by density functional theory (DFT) at the D3/PBE0/def2-TZVPP level of theory. Starting from the analogous [Fe(TREN-CAM]³⁻ crystal structure published previously, the geometry of the Sb(V) complex was first optimized. (Stack, T. D. P. et al., J. Am. Chem. Soc. 1992, 114, 1512-1514.) Key structural parameters of the DFT-optimized complex (FIG. 11D) align well with those determined from EXAFS measurements, supporting the accuracy of the model. Namely, the 6 catecholate oxygen donor atoms of the optimized TREN-CAM structure were arranged in a distorted octahedral configuration about the metal center, giving rise to DFT-predicted Sb—O and Sb—C bond lengths of 1.99 Å and 2.77 Å, respectively. With this model in hand, the Gibbs free energy change was calculated next, ΔG_{aq}, for the displacement of six hydroxides from the Sb(V) ion by TREN-CAM according to the following equation:

Sb(OH)₆⁻+H₆TREN-CAM [symbol] Sb(TREN-CAM)⁻+6H₂O

This calculation gave a negative ΔG_{aq} value of −14.53 kcal/mol, confirming that transchelation of the hydroxide ligands by TREN-CAM is thermodynamically favorable in aqueous solution. Finally, natural bond orbital (NBO) calculations were performed to elucidate the nature of Sb—O bonding responsible for the favorable chelation of Sb(V) by TREN-CAM. Natural population analysis indicates the involvement of the vacant 5s and 5p orbitals of Sb in bonding, as they were substantially populated (0.87 electrons |e| and 1.19|e|, respectively). This degree of charge transfer from the ligand to Sb(V) strengthened the dative σ-type Sb—O bonds (FIG. 11D), as evident by the relatively high calculated Wiberg bond index (0.48), which served as a measure of bond order. Additionally, beyond the Sb—O interactions in the first coordination sphere, DFT calculations also revealed the presence of outer-sphere intramolecular hydrogen bonding between the amide N—H and the ortho-phenolate oxygens of TREN-CAM in the Sb(V) complex. Likewise, hydrogen bonding was also noted in the DFT-optimized free ligand structure. Although weak, these interactions likely played an important role in stabilizing the Sb(V) complex by increasing the preorganization of the catechol units and overall rigidity of the linear chelator scaffold. Collectively, the results of these structural studies support the notion that TREN-CAM is a suitable chelator for the Sb(V) ion.

### Complex Stability and Lipophilicity

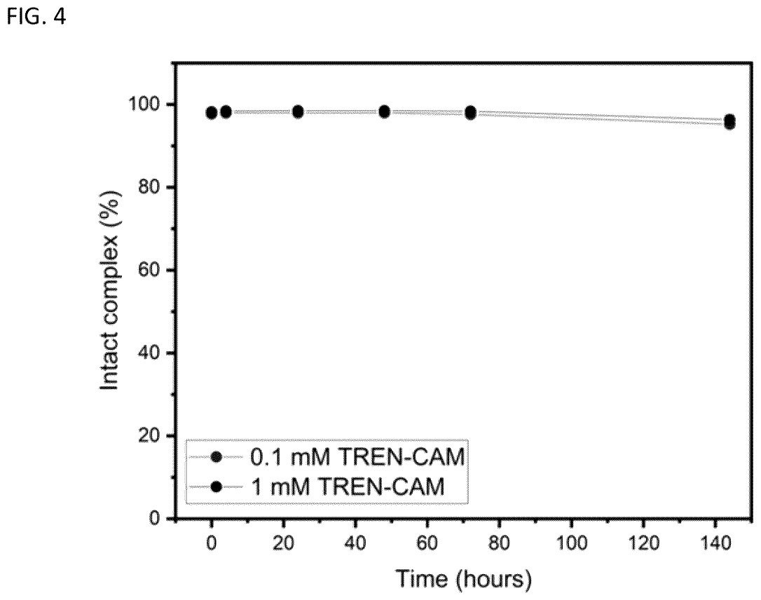

Having established that TREN-CAM can bind ^{1XX}Sb(V) in aqueous solution, the kinetic stability of the resulting complex was evaluated next. This property is extremely important when considering the use of a radioactive metal or metalloid complex for nuclear medicine applications because any release of the radioactive ion in vivo can give rise to off-target toxicity to normal tissues. As an indicator of physiological stability, the [^{1XX}Sb]Sb-TREN-CAM complex was incubated in 75% human serum at pH˜7.4 and its stability was measured over the course of several days. Samples contained a final activity of 0.14 KBq/μL and a chelator concentration of either 0.1 mM or 1 mM, keeping consistent with the radiolabeling studies. At predetermined time points, aliquots of the reaction mixtures were analyzed by radio-TLC using the method described above. Under these conditions, >95% of the ^{1XX}Sb complex remained intact for up to 6 d, well over two half-lives of ¹¹⁹Sb (FIG. 4).

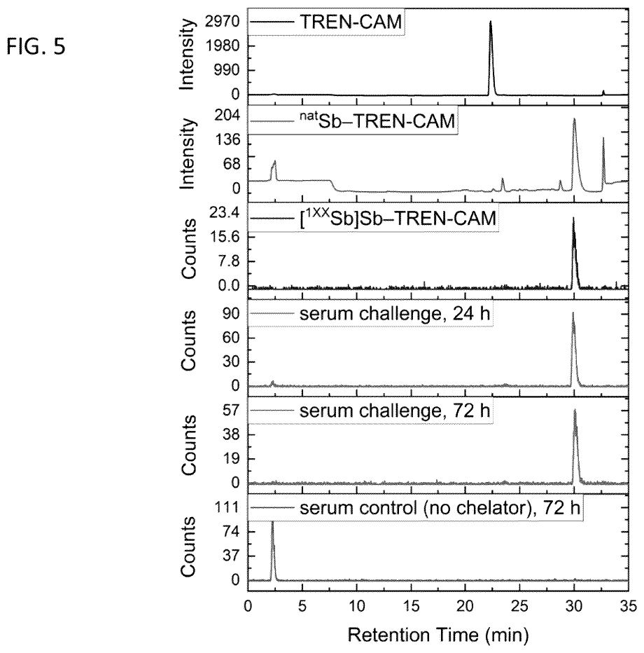

These results were subsequently cross-validated using reverse-phase radio-HPLC (FIG. 5). To overcome the issue of irreversible retention of unchelated ^{1XX}Sb on the C₁₈ column, an HPLC method was optimized in which citric acid (100 mM, pH 4.5, 0-5 min) was used first to elute free ^{1XX}Sb as the Sb(V)-citrate complex. The mobile phase was then switched to H₂O with 0.1% formic acid to wash out the citric acid (5-10 min), followed by a linear gradient to 100% CH₃OH (0.1% formic acid) to elute the Sb-TREN-CAM complex (10-30 min). Shown in FIG. 5, the retention time of radiolabeled [^{1XX}Sb]Sb-TREN-CAM (29.8 min) matched that of a ^{nat}Sb-TREN-CAM standard, confirming radiolabeling prior to adding serum. After 24, 48, and 72 h, human serum samples were aliquoted and diluted with an equal volume of acetonitrile to precipitate out the serum proteins prior to HPLC analysis. Under these conditions, greater than 96% of [^{1XX}Sb]Sb-TREN-CAM remained intact over 72 h in serum. These results are consistent with the radio-TLC analysis and indicate high kinetic stability for [^{1XX}Sb]Sb-TREN-CAM under biologically relevant conditions.

Finally, log D_{7.4} measurements for [^{1XX}Sb]Sb-TREN-CAM were made to assess the complex's lipophilicity, a property which oftentimes influences the biodistribution of radiolabeled constructs. This study was executed by partitioning [^{1XX}Sb]Sb-TREN-CAM between n-octanol and phosphate buffered saline (PBS) for 15 min before analyzing the activity of each phase by high purity germanium (HPGe) gamma spectroscopy. A log D_{7.4} value of 0.299+/−0.049 (n=3) was obtained, revealing that the Sb(V) complex of TREN-CAM is highly lipophilic.

### In Vivo Imaging, Biodistribution, and Metabolite Analysis

Mouse imaging and biodistribution studies were conducted according to IACUC approved animal protocols.

For in vivo imaging and stability studies, ¹¹⁷Sb was produced in 0.5 M NH₄OAc at the University of Wisconsin-Madison and experiments were conducted the same day. 2 mL of ¹¹⁷Sb in 0.5 M NH₄OAc pH 4 solution was combined with 220 μL of 10 mM TRENCAM in 20% DMSO solution and heated to 80° C. for 1 h. Quantitative radiolabeling was confirmed via TLC. A 1:5 dilution of the radiolabeling solution with DI H₂O was performed before passing it through a C8 Sep-Pak® cartridge (Waters, Milford, MA, USA), preconditioned with 5 mL EtOH and 5 mL H₂O) to trap the [¹¹⁷Sb]Sb-TRENCAM complex. The cartridge was rinsed with 1 mL H₂O to remove NH₄OAc buffer and unchelated ^{117/119}Sb, and then the [¹¹⁷Sb]Sb-TRENCAM complex was eluted with EtOH and collected dropwise. Purified [^{117/119}Sb]Sb-TRENCAM was diluted with 800 μL PBS and placed under a stream of N₂ to remove the EtOH. According to thermodynamic predictions based on the hydrolysis constant of 2.72 for Sb(OH)₅, the speciation of Sb(V) at pH 7.4 is likely to be Sb(OH)₆⁻. (Filella, M. et al., Earth-Sci. Rev. 2002, 59, 265-285; Baes, C. F.; Mesmer, R. E. The Hydrolysis of Cations; Wiley: New York, 1976.) Thus, unchelated ¹¹⁷Sb prepared in PBS for injection into control mice was presumably in this form.

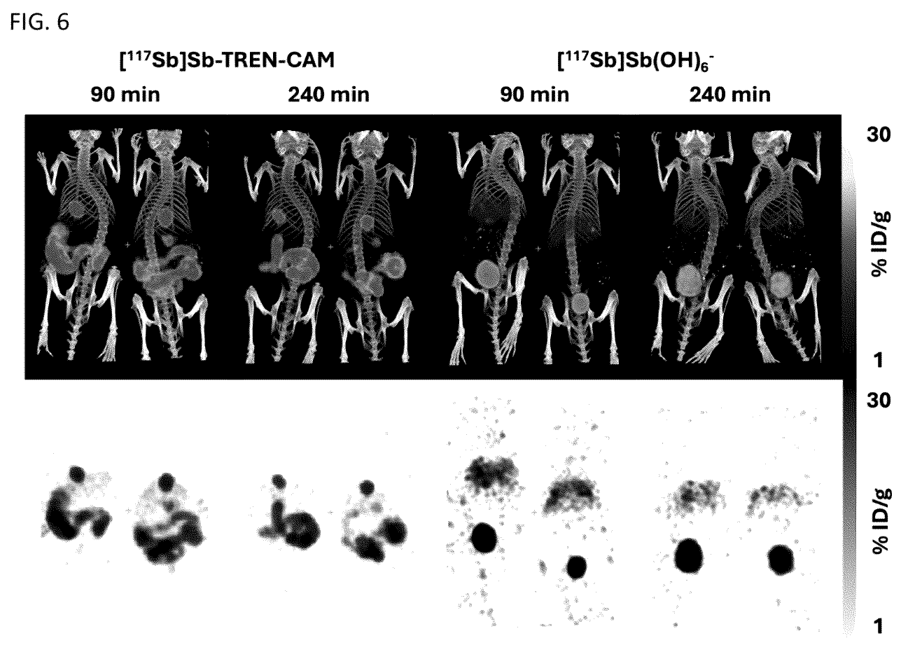

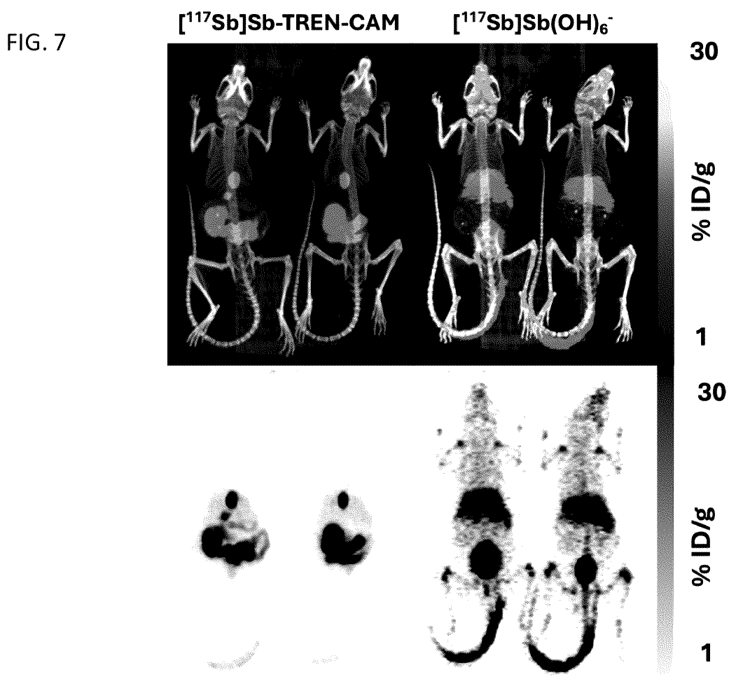

Two groups of healthy 3-month-old male BALB/c mice (n=3) (Jackson River Laboratories) were injected with 150 μL ¹¹⁷Sb solutions. The control, unchelated Sb group received 12.51 MBq±0.04 MBq (338.0 μCi±1.0 μCi) ¹¹⁷Sb, and the [¹¹⁷Sb]Sb-TRENCAM group received 8.22 MBq±0.02 MBq (222.2 μCi±0.5 μCi) injected ¹¹⁷Sb activity. SPECT/CT images were collected for 30 min using a MILabs U-SPECT/CTUHR at t=20 min, 90 min, and 240 min post injection. (FIG. 6). For SPECT imaging, all 3 mice were imaged simultaneously in the prone position under isoflourine anesthesia (3% induction, 1.5% maintenance). SPECT image reconstructions windowed both ¹¹⁷Sb (159 keV) and ¹¹⁹Sb (˜25 keV) emissions. 20 min PET/CT images (time window 3.432 ns; energy window 350-650 keV) of two mice in the prone position were collected 150 min post injection using a Seimens Inveon micro-PET/CT under isoflourine anesthesia. (FIG. 7). In both cases, scanner calibration occurred by imaging vials of ¹¹⁷Sb activity and correlating image signal strength to activity measured via High Purity Germanium (HPGe) gamma spectroscopy. CT images collected after each SPECT or PET image allowed attenuation-correction and anatomical co-registration. 3-D ordered subset expectation maximization/maximum a posteriori algorithm (OSEM3D/MAP) with non-scatter correction allowed reconstruction of CT attenuation-corrected PET images. SPECT images were reconstructed with a similarity regulated OSEM algorithm with 4 iterations, 0.8 mm voxel size, and 1.4 mm FWHM gaussian blurring. Using co-registered CT images, manual volume-of-interest delineation of tissues allowed activity biodistribution measurement, as expressed as percent injected activity per gram of tissue (% IA/g), using either Inveon Research Workstation (Siemens) for PET VOI analysis or Imalytics 3.0 (MILabs) for SPECT VOI analysis.

### Ex Vivo Biodistribution.

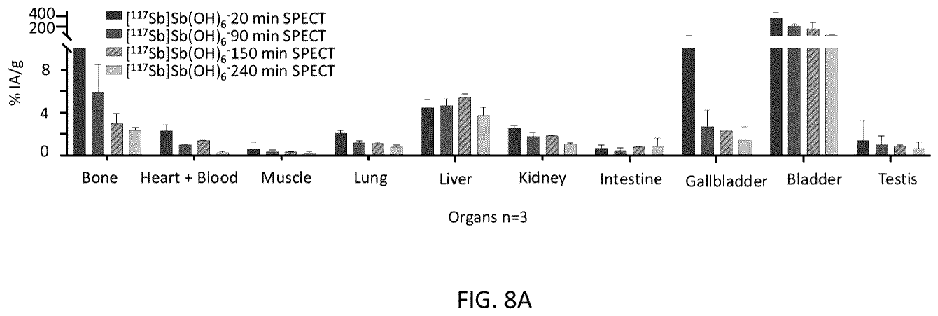

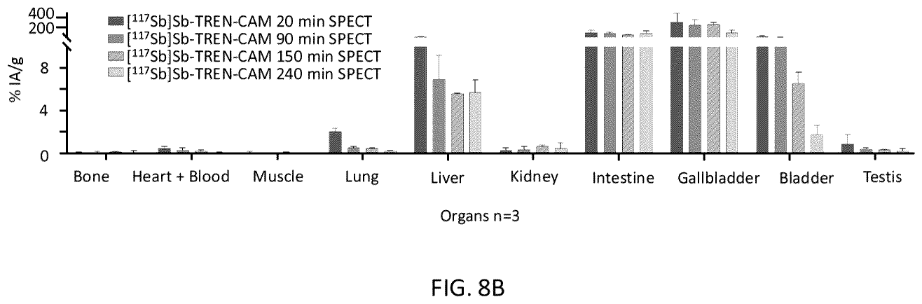

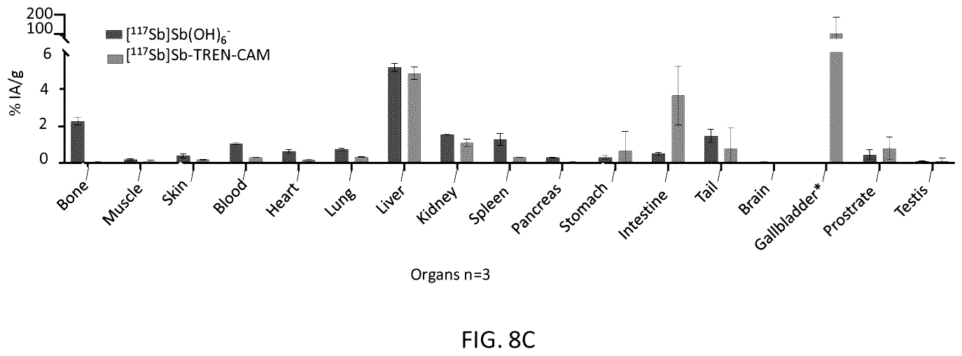

5 h post injection, animals were sacrificed using CO₂ asphyxiation with cardiac exsanguination secondary euthanasia confirmation. Tissues and organs were harvested and weighed, and their ¹¹⁷Sb activity was counted via a PerkinElmer gamma counter (Waltham, MA, USA) for ex vivo biodistribution measurement. (FIGS. 8A-8C).

### Mouse Imaging and Biodistribution.

Both imaging modalities show [¹¹⁷Sb]Sb(OH)₅ clearing through the bladder, depicting renal clearance with liver accumulation. With greater sensitivity, PET images show uptake of [¹¹⁷Sb]Sb(OH)₅ in bone. By contrast, both PET and SPECT show [¹¹⁷Sb]Sb-TRENCAM highest uptake within the gallbladder and intestines, indicative of hepatobiliary clearance.

Ex vivo biodistribution measurements confirm in vivo imaging observations (FIGS. 8A-8C), with lower bone and spleen uptake and higher intestinal and gallbladder uptake for mice administered [¹¹⁷Sb]Sb-TREN-CAM versus [¹¹⁷Sb]Sb(OH)₆⁻. In a separate study, the gallbladders were excised from mice 30 min post-injection of [¹¹⁷Sb]Sb-TREN-CAM, and their contents were collected and analyzed by radio-TLC. Metabolized gallbladder contents migrated with an R_{f} of 0.752±0.045 (n=3, uncertainty expressed as standard deviation of measurements), which is similar to the R_{f} of 0.811±0.005 (n=3) for unmetabolized [¹¹⁷Sb]Sb-TREN-CAM. These results, in conjunction with the markedly different clearance routes of free versus complexed ¹¹⁷Sb revealed by the imaging studies, demonstrate that the [¹¹⁷Sb]Sb-TREN-CAM complex remains intact over the time course of the study. Thus, these findings mark the first observation of a stable radio-Sb complex in vivo. Furthermore, the success of SPECT and PET imaging with ¹¹⁷Sb highlight this radioisotope as a useful diagnostic partner to ¹¹⁹Sb.

### Additional Experimental Details

### 1.1 Materials and Instrumentation

All reagents and solvents were purchased from commercial vendors and used without further purification. All solutions were prepared with 18 MΩ*cm deionized water, unless otherwise noted. 1,4,7,10-Tetraazacyclododecane-N,N′,N″,N″′-tetraacetic acid (DOTA, min. 98%) was purchased from Strem (Newburyport, MA, USA). Deferoxamine mesylate (DFO, 95%) and SbCl₅ (99.999% metals basis, ultra-dry) were purchased from Thermo Scientific Chemicals (Waltham, MA, USA). SbCl₃ (>99.95% trace metals grade) was purchased from Sigma-Aldrich (St. Louis, MO, USA). Citric acid was supplied by either Avantor Sciences (Radnor, PA, USA) or Honeywell (Fluka, TraceSELECT, ≥99.9998%; Charlotte, NC, USA). Phosphate buffered saline (PBS) was supplied by either Lonza (Walkersville, MA, USA) or Thermo Scientific Chemicals (ultrapure, molecular biology grade).

### 1.2 TREN-CAM Synthesis

TREN-CAM was prepared as previously described. (Joaqui-Joaqui, M. A.; Pandey, M. K.; Bansal, A.; Raju, M. V. R.; Armstrong-Pavlik, F.; Dundar, A.; Wong, H. L.; Degrado, T. R.; Pierre, V. C. Catechol-Based Functionalizable Ligands for Gallium-68 Positron Emission Tomography Imaging. Inorg. Chem. 2020, 59 (17), 12025-12038.) The crude TREN-CAM product was further purified by reversed-phase flash chromatography (RediSep Rf Gold C18Aq column) on a CombiFlash Rf+ system (25% to 100% gradient of MeOH in H₂O with 0.1% trifluoroacetic acid, TFA). Pure fractions were identified by analytical high-performance liquid chromatography (HPLC, Shimadzu) using a Restek Ultra Aqueous C₁₈ column (100 Å, 5 μm, 250 mm×4.6 mm). Method: 0-5 min, 10% MeOH/H₂O with 0.1% TFA; 5-25 min, 10% to 100% linear gradient of MeOH/H₂O with 0.1% TFA. These fractions were combined, concentrated, and then lyophilized to give the title compound as a white solid.

TREN-CAM·1TFA·1H₂O: Based on the peak integrations in the ¹H and ¹⁹F NMR spectra of this compound relative to an internal standard of fluorobenzene, in conjunction with elemental analysis, TREN-CAM was isolated as a TFA salt with one water of hydration. ¹H NMR (400 MHz, CD₃OD): δ=7.10-7.07 (dd, 3H, J=8.0 Hz, 4.0 Hz), 6.91-6.89 (dd, 3H, J=8.0 Hz, 4.0 Hz), 6.63-6.61 (t, 3H, J=8.0 Hz), 3.84 (t, 6H, J=6.0 Hz), 3.65 (t, 6H, J=6.0 Hz). ¹³C{¹H} NMR (100 MHz, CD₃OD): δ=172.7, 149.6, 147.1, 120.0, 119.9, 119.6, 116.5, 55.5, 36.0. HPLC t_{R}=18.85 min. HRMS (ESI-TOF, positive mode): m/z calcd for [C₂₇H₃₀N₄O₉+H]⁺, 555.2086; found, 555.2108. Elemental analysis calcd for C₂₇H₃₀N₄O₉·CF₃COOH·H₂O: C, 50.73; H, 4.84; N, 8.16; F, 8.30. Found: C, 50.39; H, 4.88; N, 8.10; F, 8.10.

### 1.3 Target Preparation and Irradiation

The University of Wisconsin-Madison (UWM) Cyclotron research group owns and operates a GE PETtrace (GE Healthcare systems, Uppsala, Sweden) capable of accelerating 16 MeV protons and 8 MeV deuterons, which produce radioantimony (radio-Sb) primarily via (p,n) and (d,n) nuclear reactions upon tin targets. Natural tin was electroplated upon gold target backings using a sulfuric acid-based electrolytic solution as previously reported. (Olson, A. P.; Ma, L.; Feng, Y.; Najafi Khosroshahi, F.; Kelley, S. P.; Aluicio-Sarduy, E.; Barnhart, T. E.; Hennkens, H. M.; Ellison, P. A.; Jurisson, S. S.; Engle, J. W. A Third Generation Potentially Bifunctional Trithiol Chelate, Its Nat, 1XX Sb(III) Complex, and Selective Chelation of Radioantimony (119 Sb) from Its Sn Target. Inorg. Chem. 2021, 60, 15223-15232.) With ten naturally occurring stable tin isotopes, bombardment of naturally enriched tin with different particles or particles at different energies will create a different profile of radio-Sb isotopes. These different production profiles can be advantageously used according to the desired application. Produced radio-Sb isotopes, their half-lives, and primary emissions are recorded in Table 1 in the main text.

To allow for sufficient time to execute radiolabeling and in vitro stability experiments, production of a higher proportion of longer-lived Sb radioisotopes is desired. This production was achieved using 16 MeV proton irradiation of targets with lineal mass densities of 500-600 mg/cm² at a current of 30 μA for 2 h, which initiated ^{nat}Sn(p,n)^{1XX}Sb nuclear reactions. Proton-irradiated targets were allowed to decay for 12 h before removal from the cyclotron to reduce exposure of personnel to radiation arising from short-lived radionuclides.

For in vivo mouse imaging and biodistribution studies, production of the radioisotopic imaging analogue ¹¹⁷Sb in high yield and radionuclidic purity is desired, while limiting the formation of longer-lived radioisotopes. This balance was achieved via 8 MeV deuteron bombardment of 180-200 mg/cm² targets at 35 μA for 1.5 h, which initiated ^{nat}Sn(d,n)^{1XX}Sb reactions. Targets were allowed to decay for 45 min before removal from the cyclotron to decrease exposure of personnel to radiation arising from extremely short-lived by-products (t_{1/2}<5 min).

A comparison between the proportion of radioantimony isotopes produced from proton versus deuteron bombardment can be found in Table 2.

TABLE 2
Structural parameters of Sb(V) coordination environment
obtained from fitting Sb K-edge EXAFS spectra.

| | KSb(OH)₆ | [Sb(TREN-CAM)]⁻ | [Sb(Catechol)₃]⁻ |
|---|---|---|---|
| CN: Sb—O | 6.3 ± 0.3 | 6.7 ± 0.4 | 6.8 ± 0.4 |
| R: Sb—O (Å) | 1.980 ± 0.004 | 1.992 ± 0.005 | 1.993 ± 0.005 |
| σ²: Sb—O (Å²) | 0.0022 ± 4 × 10⁻⁴ | 0.0043 ± 7 × 10⁻⁴ | 0.0040 ± 6 × 10⁻⁴ |
| CN: Sb—C | — | 4 ± 2 | 6 ± 3 |
| R: Sb—C (Å) | — | 2.79 ± 0.02 | 2.78 ± 0.02 |
| σ²: Sb—C (Å²) | — | 0.006 ± 0.005 | 0.007 ± 0.006 |

### 1.4 Sn/Sb Separation Chemistry

Modification to a previously reported liquid-liquid extraction procedure allowed for separation of radio-Sb from bulk tin target material. (Kostelnik, T. I.; Olson, A. P.; Grundmane, A.; Ellison, P. A.; Mynerich, J.; Chen, S.; Marinova, A.; Randhawa, P.; Karaivanov, D.; Aluicio-Sarduy, E.; Barnhart, T. E.; Orvig, C.; Ramogida, C. F.; Hoehr, C.; Filosofov, D.; Engle, J. W.; Radchenko, V. Production and Radiochemistry of Antimony-120m: Efforts toward Auger Electron Therapy with 119Sb. Nucl. Med. Biol. 2023,122-123, 108352.) Briefly, irradiated tin targets were dissolved in glass vials under N₂ gas using c.HCl (2 mL) at 90° C. for 1 h. The resulting solution was removed from heat and allowed to cool at 25° C. for 15 min. Following cooling, c.HCl (200 μL) and 30% H₂O₂ (100 μL) was added to oxidize any Sb(III) to Sb(V). The solution was then contacted with an equal volume of dibutyl ether (DBE), allowing non-polar SbCl₅ to extract into the organic layer. (Bonner, N. A. The Exchange Reaction between Antimony (III) and Antimony (V) in Hydrochloric Acid Solutions. J. Am. Chem. Soc. 1949, 71 (12), 3909-3914.) Organic and aqueous layers were separated, and the organic layer was washed with 1.5 mL c.HCl to remove any co-extracted tin. ^{1XX}Sb(V) was back-extracted into aqueous solution with 2 mL volume of either H₂O, PBS, or 0.5 M NH₄OAc (pH 6) and used in subsequent experiments.

### 1.4 ^{1XX}Sb Oxidation State Studies

^{1XX}Sb was produced and purified at UWM, with back-extraction into H₂O, and then shipped to ORNL. Upon arrival, the oxidation state of the ^{1XX}Sb was analyzed by anion-exchange radio-HPLC (Shimadzu instrument; LabLogic Flow-Ram detector; Hamilton PRP-X100 column, 150×4.1 mm; 5 μm) using a method established in the literature for non radioactive Sb. (Hansen, C.; Schmidt, B.; Larsen, E. H.; Gammelgaard, B.; Stürup, S.; Hansen, H. R. Quantitative HPLC-ICP-MS Analysis of Antimony Redox Speciation in Complex Sample Matrices: New Insights into the Sb-Chemistry Causing Poor Chromatographic Recoveries. Analyst 2011, 136 (5), 996-1002.) Specifically, ^{1XX}Sb (30 μL) was diluted to an activity concentration of 0.059 kBq/μL (0.016 μCi/μL) with 0.5 M NH₄OAc (pH 4, 120 μL). The sample was injected onto the anion exchange column with and without pre-mixing it first with an equal volume of 0.1 M ammonium citrate (pH 4.5). HPLC chromatograms were obtained using an isocratic method of 0.1 M ammonium citrate (pH 4.5, 1 mL/min, 35 min). The fractions were collected and gamma counted (Perkin Wizard2 gamma counter, open window). For both samples, the total count rate summed across fractions was found to be nearly identical to the count rate measured from aliquots that were not subjected to HPLC. These results indicate that full recovery of ^{1XX}Sb from the anion exchange column is achieved using the elution method noted, regardless of whether samples are mixed with ammonium citrate prior to injection. However, a single peak (t_{R}=3 min) was observed for the sample that was mixed with an aliquot of ammonium citrate prior to injection, whereas the sample without ammonium citrate displayed two peaks in the chromatogram (t_{R}=1.5 min and 3 min). The peak at 3 min has been assigned in the literature as an Sb(V)-citrate complex. (Hansen, et al. (2011)) As such, we postulate that the peak at 1.5 min is an Sb(V)-acetate species arising from the use of NH₄OAc buffer.

To further determine whether a change in oxidation state occurs upon heating, the experiment was repeated (0.0038 μCi/μL activity concentration), but one sample was heated at 80° C. for 1 h while the other sample remained at ambient temperature. No ammonium citrate was added to the samples prior to HPLC injection. The chromatograms of these samples were identical, supporting the notion that ^{1XX}Sb remains in the same oxidation state under these experimental conditions.

Finally, as validation that the species eluting at ˜3 min can be unequivocally assigned to an Sb(V) species, we explored the ability of various oxidizing and reducing agents to alter the retention time of ^{1XX}Sb on the anion exchange column. For this experiment, partially oxidized ^{1XX}Sb was used as a control, which displayed peaks at both 3 min and 8 min after dilution with an equal volume of ammonium citrate prior to injection. Samples were prepared as follows:

H₂O₂: 20 μL of ^{1XX}Sb (570 nCi), 90 μL of 0.5 M NH₄OAc (pH 4), 40 μL of H₂O₂ (50 wt %)

Iodobead: 20 μL of ^{1XX}Sb (570 nCi), 130 μL of 0.5 M NH₄OAc (pH 4), 1 iodobead (Pierce iodination bead, Thermo Scientific)

Mercaptoacetic acid: 20 μL of ^{1XX}Sb (570 nCi), 130 μL of 0.5 M NH₄OAc (pH 4), mercaptoacetic acid (10 μL in H₂O, 7.5 μmol)

Samples were mixed for 24 h at 25° C. (unoptimized), and then aliquots were withdrawn, diluted with ammonium citrate, and analyzed by radio-anion exchange HPLC. Under these conditions, nearly all the ^{1XX}Sb eluted at 3 min when treated with H₂O₂ or an iodobead, whereas ^{1XX}Sb eluted at 8 min when treated with mercaptoacetic acid. These results support the assignment of the 3 min peak as ^{1XX}Sb(V) and the 8 min peak as^{1XX}Sb(III), consistent with anion exchange studies carried out with nonradioactive Sb. (Hansen, et al. (2011))

### Analytical HPLC:

ORNL cold HPLC: 14.8 min (Peak 1), 21.3 min (Peak 2)
   Instrument: Shimadzu. Column: Restek Ultra Aqueous C₁₈ column (100 Å, 5 μm, 250 mm×21.2 mm). Flow rate: 1 mL/min. Solvents: A=0.1% FA/H₂O, B=0.1% FA/MeCN. Method: 0-5 min: 10% B; 5-25 min: linear ramp 10-100% B; 25-30 min: 100% B.
ORNL radio-HPLC: 22.8 min (Peak 1), 35.7 min (Peak 2)
   Instrument: Shimadzu coupled to a LabLogic Flow-Ram detector equipped with Laura software. Column: Restek Ultra Aqueous C₁₈ column (100 Å, 5 μm, 250 mm×21.2 mm). Flow rate: 1 mL/min. Solvents: A=0.1 M ammonium citrate (pH 4.5), B=0.1% FA/H₂O, C=0.1% FA/MeCN. Method: 0-5 min: 100% A; 5-10 min: 100% B; 10-35 min: linear ramp to 0% B/100% C; 35-40 min: 100% C; 40-46 min, 100% A.
UWM radio-HPLC: 23.7 min (Peak 1), 30.0 min (Peak 2)

Instrument: Agilent 126011 system (Santa Clara, CA) with Ortec (AMETEK ORTEC, Oak Ridge, TN) detector. Column: 150 mm C18 Jupitor column (Phenomenex, Torrance, CA). Flow rate: 1 mL/min. Solvents: A=0.1 M ammonium citrate (pH 4.5), B=0.1% FA/H₂O, C=0.1% FA/MeOH. Method: 0-5 min: 100% A; 5-10 min: 100% B; 10-30 min: linear ramp to 0% B/100% C; 30-32 min: 100% B; 32-37 min, 100% A.

### NMR/HRMS:

Peak 1: HRMS: m/z 669.0592; calcd for [C₂₇H₂₄N₄O₉Sb]⁻: 669.0587. m/z 705.0802; calcd for [C₂₇H₂₄N₄O₉Sb+2H₂O]⁻: 705.0798.
Peak 2: ¹H NMR (400 MHz, DMSO-d₆) δ 8.61 (t, J=4.9 Hz, 3H), 7.28 (d, J=8.3 Hz, 3H), 7.06 (d, J=7.9 Hz, 3H), 6.80 (t, J=8.1 Hz, 3H), 6.54 (s, 2H), 3.50 (br s, 6H), 2.50 (br s, 6H, overlapped with residual DMSO peak). ¹³C{¹H} NMR (101 MHz, DMSO-d₆) δ 163.8, 145.9, 144.8, 120.5, 119.0, 118.8, 116.0, 34.6. HRMS: m/z 669.0592; calcd for [C₂₇H₂₄N₄O₉Sb]⁻: 669.0587.

### 1.5 X-ray Absorption Spectroscopy (XAS)

### 1.5.1 Sample Preparation

XAFS samples of Sb(V) in the absence of chelator were prepared as 0.7 mM Sb₂O₅ in 1 M HClO₄ (pH 1), 7 mM KSb(OH)₆ in pure water (pH 7), 4 mM KSb(OH)₆ in 0.01 M NaOH+HCl (pH 9), and 4 mM KSb(OH)₆ in 0.01 M NaOH (pH 12). Samples containing Sb(V) (7 mM) and catechol (300 mM) were prepared in water using KSb(OH)₆, as described previously by others. (Tella, M.; Pokrovski, G. S. Antimony (V) Complexing with O-Bearing Organic Ligands in Aqueous Solution: An X-Ray Absorption Fine Structure Spectroscopy and Potentiometric Study. Mineral. Mag. 2008, 72 (1), 205-209 and Tella, M.; Pokrovski, G. S. Stability and Structure of Pentavalent Antimony Complexes with Aqueous Organic Ligands. Chem. Geol. 2012, 292-293, 57-68.) The pH of the samples was adjusted to 9, 4, and 3, respectively, with NH₄OH. Likewise, a sample containing TREN-CAM (14 mM) and KSb(OH)₆ (1 mM) was prepared in water and adjusted to pH 9 with NH₄OH.

### 1.5.2 Measurements

X-ray absorption spectroscopy measurements were performed at the Sb Kedge at beamline 8-ID of the National Synchrotron Light Source II (NSLS II). Solid Sb samples (Sb₂O₃ and Sb₂O₅ standards) were prepared as pellets, whereas Sb solution samples were placed in Kapton capillaries (1.8 mm inner diameter, 0.05 mm thickness, Cole-Parmer) and sealed with epoxy. All data were acquired in the fluorescence geometry. The data were energy-calibrated to the first derivative maximum of an Sb foil defined at 30491 eV. Data normalization was performed using the Athena software package. (Ravel, B.; Newville, M. ATHENA, ARTEMIS, HEPHAESTUS: Data Analysis for X-Ray Absorption Spectroscopy Using IFEFFIT. J. Synchrotron Radiat. 2005, 12 (4), 537-541.) Ejected photoelectrons are defined by their wavenumber (k) in relation to the absorption edge energy (E₀) through Equation S1:

$$k = \sqrt{2 {m}_{e} ( E - {E}_{0} ) / {ℏ}^{2}} \qquad ( S1 )$$

The experimental EXAFS oscillations of each sample, χ(k), are extracted from the normalized XAS data using subtraction of a spline and a cutoff distance (R_{BKG}) of 1.0 Å. For analysis of the EXAFS region, we use the EXAFS relationship given by Equation S2:

$$χ ( k ) = {\sum }_{i} \frac{{F}_{i} ( k ) {S}_{0}^{2} {N}_{i}}{{kR}_{i}^{2}} {e}^{- 2 {k}^{2} {σ}_{i}^{2} {e}^{\frac{- 2 {R}_{i}}{λ ( k )}}} sin ( 2 {kR}_{i} + {δ}_{i} ( k ) - \frac{4}{3} {k}^{3} {C}_{3 , i} ) \qquad ( S2 )$$

where the index, i, is considered the path index and the χ(k) is calculated as the summation over all paths. For fitting of the EXAFS spectra, FEFF6 within the Artemis software package. was used with data weighted by k². In eq. S2, Fᵢ(k), δᵢ(k), and λ(k) represent the effective scattering amplitude, total phase shift, and mean-free-path of the photoelectron and each are derived from FEFF6. The many-body amplitude-reduction factor, S₀², is fixed to 0.95. Therefore, the parameters still to be fit include, Nᵢ, the degeneracy of the path (and therefore the coordination number for single scattering paths); Rᵢ, the half-path length; σ²ᵢ, the Debye-Waller factor; and C_{3,i}, the asymmetry of the distribution. Variation of C_{3,i} was found to provide negligible improvements on the single scattering paths and thus was not included in the fitting process. Additionally, a single non-structural parameter for all paths, ΔE₀, is varied to align the k=0 point of the experimental data and theory. All scattering paths were generated utilizing the DFT-optimized Sb-TREN-CAM model structure (see Section 1.6) and included the first and second scatter shells surrounding the Sb atom (O and C paths respectively). Multiple scattering paths (Sb—O—C—Sb & Sb—C—O— Sb) are also included into the fitting procedure and were found to give a slight improvement in the fitting statistics. This approach allowed the number of variables (7) per fit to stay below the number of independent data points (14).

### 1.6 Quantum Chemical Calculations

Electronic structure calculations were performed with the Gaussian 16 C.01 software. We adopted the density functional theory (DFT) approach for our calculations using the PBE0 density functional with the def2-TZVPP basis set and the associated effective core potential for the Sb atom. (Adamo, C.; Barone, V. Toward Reliable Density Functional Methods without Adjustable Parameters: The PBE0 Model. J. Chem. Phys. 1999,110 (13), 6158-6170 and Weigend, F.; Ahlrichs, R. Balanced Basis Sets of Split Valence, Triple Zeta Valence and Quadruple Zeta Valence Quality for H to Rn: Design and Assessment of Accuracy. Phys. Chem. Chem. Phys. 2005, 7, 3297-3305.) The DFT-D3 approach of Grimme was used to account for the dispersion interactions. (Grimme, S.; Antony, J.; Ehrlich, S.; Krieg, H. A Consistent and Accurate Ab Initio Parametrization of Density Functional Dispersion Correction (DFT-D) for the 94 Elements H-Pu. J. Chem. Phys. 2010, 132 (15), 154104.) Frequency calculations were performed at the same level of theory to ensure that the optimized geometries were minima and to compute zero-point energies and thermal corrections. Using the gas-phase geometries, implicit solvent corrections were obtained at 298.15 K with the SMD solvation model. (Marenich, A. V.; Cramer, C. J.; Truhlar, D. G. Universal Solvation Model Based on Solute Electron Density and on a Continuum Model of the Solvent Defined by the Bulk Dielectric Constant and Atomic Surface Tensions. J. Phys. Chem. B 2009, 113 (18), 6378-6396.) The geometry for the Sb(TREN-CAM)⁻ complex was optimized starting from the single crystal structure. For the TREN-CAM ligand, different conformers were considered, and the results are reported using the lowest energy configuration identified at the DFT-D3/PBE0/def2-TZVPP level.

Complexation free energy in aqueous solution, ΔG_{aq}, was calculated using the thermodynamic cycle methodology described previously. (Lashley, M. A.; Ivanov, A. S.; Bryantsev, V. S.; Dai, S.; Hancock, R. D. Highly Preorganized Ligand 1,10-Phenanthroline-2,9-Dicarboxylic Acid for the Selective Recovery of Uranium from Seawater in the Presence of Competing Vanadium Species. Inorg. Chem. 2016, 55 (20), 10818-10829 and Ivanov, A. S.; Bryantsev, V. S. Assessing Ligand Selectivity for Uranium over Vanadium Ions to Aid in the Discovery of Superior Adsorbents for Extraction of UO₂²⁺ from Seawater. Dalton Trans. 2016, 45 (26), 10744-10751.) Standard state correction terms were introduced, which are defined using different standard state conventions. In particular, the free energy change for the conversion of 1 mol of solute from the gas phase at a standard state of 1 atm (24.46 L/mol) to the aqueous phase at a standard state of 1 mol/L at 298.15 K is given by ΔG^{o→}*=1.89 kcal/mol. Likewise, RT ln([H₂O])=2.38 kcal/mol at 298.15 K, is the free energy change for the conversion of 1 mol of solvent from the aqueous phase at 1 mol/L to pure water at a standard state of 55.34 mol/L.

The chemical bonding analysis in the optimized Sb(TREN-CAM)⁻ complex was carried out at the DFT-D3/PBE0/def2-TZVPP level of theory using the NBO methodology, (Glendening, E. D.; Landis, C. R.; Weinhold, F. Natural Bond Orbital Methods. WIREs Comput. Mol. Sci. 2012, 2, 1-42) as implemented in the NBO7 program. (Glendening, E. G.; Badenhoop, J. K.; Reed, A. E.; Carpenter, J. E.; Bohmann, J. A.; Morales, C. M.; Karafiloglou, P.; Landis, C. R.; Weinhold, F. NBO 7.0, 2018 and Glendening, E. D.; Landis, C. R.; Weinhold, F. NBO 7.0: New Vistas in Localized and Delocalized Chemical Bonding Theory. J. Comput. Chem. 2019, 40 (25), 2234-2241.)

### 1.7 Radiolabeling, Serum Stability, and Log D_{7.4} Studies

### 1.7.1 General

For radiolabeling studies, end-of-bombardment (EOB) decay corrected activities of 7.1 MBq±3.4 MBq (190 μCi±93 μCi) ¹²⁰Sb, 11.7 MBq±7.7 MBq (318 μCi±208 μCi) ¹²²Sb, and 0.3 MBq±0.2 MBq (8.0 μCi±7.0 μCi)¹²⁴Sb were produced in Wisconsin and shipped to ORNL (n=5, back-extracted into H₂O), unless otherwise noted. Sub-stock solutions of ^{1XX}Sb were prepared in 0.1 M HCl, unless otherwise noted. Stock solutions of DFO and DOTA were prepared in H₂O. Stock solutions of TREN-CAM were prepared in 20% DMSO/80% H₂O for radiolabeling reactions in HCl or NH₄OAc. The concentrations of DFO and DOTA were verified by potentiometric titration. The concentration of TREN-CAM was verified by quantitative NMR (qNMR) using a known concentration of potassium hydrogen phthalate as an internal standard. Subsequently, these stocks were serially diluted with H₂O to afford a series of concentrations. NH₄OAc buffers (0.5 M, pH 4, 6) were prepared using solid NH₄OAc (99.999% trace metals basis, Sigma-Aldrich) and glacial HOAc (≥99%, Sigma-Aldrich), and were rendered metal-free by contact with Chelex (5 g/L) overnight via the batch method. Each buffer was subsequently filtered through a polypropylene column equipped with a 20 μm polyethylene frit and stored in an acid-washed plastic bottle. Human serum from male AB plasma (USA origin, sterile-filtered) was purchased from Sigma-Aldrich and used as is. Normal human serum as a lyophilized powder was purchased from Jackson ImmunoResearch (Ely, UK) and reconstituted at 2× concentration in H₂O.

### 1.7.2 Preliminary Chelator Screening

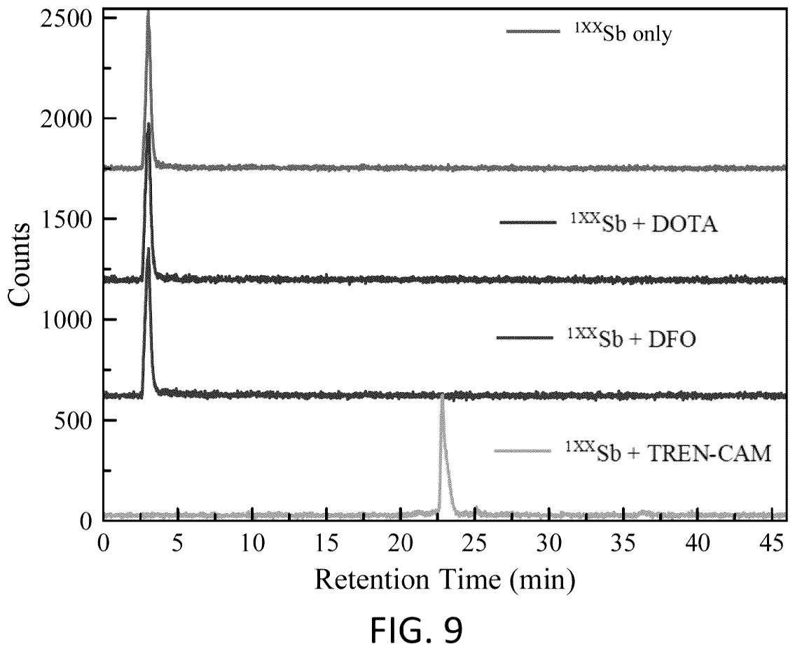

Three chelators were initially screened for their ability to complex ^{1XX}Sb: TREN-CAM, DFO, and DOTA. Radiolabeling reactions were carried out at pH 4 (0.5 M NH₄OAc) and 80° C. using 21 kBq (0.57 μCi) of ^{1XX}Sb (0.1 M HCl) and 1×10⁻³ M of chelator. The final volume of each sample was 150 μL. Control samples received water instead of chelator. After 60 min, the reactions were analyzed by radio-HPLC on a Shimadzu instrument coupled to a LabLogic Flow-Ram detector equipped with Laura software. Column: Restek Ultra Aqueous C₁₈, 1 mL/min. When using a traditional MeOH/H₂O or MeCN/H₂O gradient method with 0.1% FA, we observed a tendency for unchelated ^{1XX}Sb(V) to be retained irreversibly on the column. These results prompted us to develop the following method: A=0.1 M ammonium citrate (pH 4.5), B=0.1% FA/H₂O, C=0.1% FA/MeCN. Time program: 0-5 min: 100% A; 5-10 min: 100% B; 10-35 min: linear ramp to 0% B/100% C; 35-40 min: 100% C; 40-46 min, 100% A. Using this method, any unchelated ^{1XX}Sb elutes completely in the first 5 min. (FIG. 9)

1.7.3 Concentration-, Time-, and pH-Dependent Radiolabeling

Radio-TLC. Radiolabeling experiments were performed in triplicate at each TREN-CAM concentration by addition of chelator sub-stock (10 μL) and radionuclide (21 kBq, 0.57 μCi total activity from ^{120m}Sb, ¹²²Sb, and ¹²⁴Sb in 30 μL of 0.1 M HCl) to polypropylene screw-capped tubes containing 110 μL of either HCl (10 mM, pH˜2) or NH₄OAc buffer (pH 4 or pH 6). The final chelator concentrations ranged from 10⁻⁸ to 10⁻³ M. Control samples were also prepared in which chelator was substituted with H₂O. The samples were shaken to ensure complete mixing and then heated to 80° C. for 60 minutes using a heating block. After 60 min, 10 μL of 0.3 M oxalic acid was added to each sample. Heating at 80° C. was resumed for 10 min, after which time an aliquot (5 μL) was removed from each sample and spotted onto a TLC strip (aluminum-backed silica gel 60 RP-18, F₂₅₄, Merck). The TLC strips were developed using an oxalic acid mobile phase (0.25 M). Under these conditions, complexed radionuclide remained at the baseline and free radionuclide migrated with the solvent front. (FIG. 3) TLC imaging was performed using an AR-2000 scanner system equipped with P-10 gas and WinScan 3 imaging software (Eckert & Ziegler Radiopharma Inc). Radiochemical yields (RCYs) were calculated by dividing the counts associated with complexed radionuclide by the total counts integrated along the length of the TLC plate. The pH of representative samples was confirmed by spotting an aliquot (1 μL) onto a pH indicator strip. Separate experiments were carried out in an analogous manner to assess the effects of time (10, 30, 60, 120 min) and temperature (25, 37, 60, 80° C.) on TREN-CAM radiolabeling at a single concentration (1×10⁻³ M) and pH 4. One sample was prepared for each timepoint/temperature pair. At the designated timepoints, 10 μL of 0.3 M oxalic acid was added, and each sample was heated at 80° C. for 10 min before spotting on a TLC strip for analysis.

### 1.7.4 Serum Stability Challenges

Radio-TLC. TREN-CAM (4×10⁻³ M and 4×10⁻⁴ M) was radiolabeled in triplicate for 60 min at 80° C. with 84 kBq (2.28 μCi) combined total of ¹²⁰Sb, ¹²²Sb, ¹²⁴Sb in pH 4.0 NH₄OAc buffer (150 μL final volume). These two concentrations of TREN-CAM were selected based on the two lowest concentrations that gave quantitative or nearly quantitative RCYs in the radiolabeling experiments described above in NH₄OAc, multiplied by a factor of 4 to account for subsequent dilution by serum. Next, 10 uL of 0.3 M oxalic acid was added to all samples, and the reaction solutions were heated to 80° C. for 10 minutes. RCYs of >97% were confirmed by radio-TLC for all radiolabeling reactions. The samples at pH 4 were adjusted to pH 7.3-7.5 using 10 M NH₄OH. Next, an aliquot of human serum (450 μL) was added to each sample, yielding a final reaction volume of 610 μL and final chelator concentrations of 1×10⁻³ M or 1×10⁻⁴ M. Control samples were also prepared in which chelator was substituted with H₂O and diluted with 450 μL of serum. Samples were rotated end-over-end at 37° C. and aliquoted at 4 h, 1 d, 2 d, 3 d, and 6 d. The percentage of intact complex was determined by radio-TLC. The final pH of representative samples was determined to be 7.3-7.5 by spotting an aliquot (1 μL) of reaction solution onto a pH indicator strip.

### Radio-HPLC

For this experiment, ^{1XX}Sb (84 kBq, 2.28 μCi) was back-extracted into 0.5 M NH₄OAc (pH 6, 1 mL), which resulted in a solution with a pH of 4. To this solution was added TREN-CAM (100 μL of 10 mM stock) in 20% DMSO/H₂O. The reaction was heated for 60 min at 80° C., diluted to ˜10 mL with H₂O, and loaded onto a Waters C8 Sep-Pak cartridge pre conditioned with 5 mL of EtOH and 5 mL of H₂O. The cartridge was dried using air, and then EtOH was passed through the cartridge. The first nine drops were discarded, and the following 12 drops were collected and counted to confirm elution of >60% of the original activity. After collecting the eluate, PBS (200 μL) was added to the solution. The EtOH was removed from the solution using a stream of N₂ until no further change in mass was detected. The radiolabeled TREN-CAM solution was analyzed by radio-HPLC (UWM instrument/method) to confirm complexation and purity before adding it to an equal volume of 2× human serum. For control samples, ^{1XX}Sb was back-extracted directly into PBS. The pH was re-adjusted to 7.5 and combined with an equal volume of 2× human serum. The samples were incubated at 37° C. At t=1, 2, and 3 d (n=1 per timepoint), aliquots of human serum [^{1XX}Sb]Sb-TREN-CAM challenge solutions were diluted with an equal volume of MeCN and centrifuged, crashing and pelleting serum proteins out of the solution. The supernatant was diluted 1.5-fold with citric acid (pH 4.5, 100 mM) and then analyzed by radio-HPLC to determine the percent of intact complex remaining. Furthermore, activity in the supernatant, precipitate, and vial for each sample was measured by gamma counting.

### 1.7.5 Log D_{7.4} Measurements

TREN-CAM (1 mM) was quantitatively radiolabeled (assessed via radio-HPLC) with 450 kBq±0.8% (12 μCi) ¹²⁰Sb (uncertainty expressed as counting uncertainty) after 1 h at pH 4.0 (NH₄OAc buffer) and 80° C. (n=1). The solution was diluted 1:10 with DI H₂O and passed through a preconditioned (10 mL EtOH, 10 mL DI H₂O) C8 Sep-Pak® (Waters, Milford, MA), trapping [^{1XX}Sb]Sb-TREN-CAM. DI H₂O (1 mL) was passed through the loaded C8 cartridge to remove any residual salts, followed by EtOH (1 mL) to elute purified [^{1XX}Sb]Sb-TREN-CAM from the cartridge. 200 μL PBS was added to the purified [^{1XX}Sb]Sb-TREN-CAM eluent, and a stream of N₂ was used to remove the EtOH. In triplicate, 50 μL of purified [^{1XX}Sb]Sb-TREN-CAM in PBS was added to 950 μL PBS and 1 mL n-octanol. The samples were vortexed for 15 min, allowing [^{1XX}Sb]Sb-TREN-CAM to partition between the organic and aqueous phases. After vortexing, the extraction solution was allowed to settle and 0.5 mL aliquots from each phase were collected and analyzed for ^{120m}Sb activity by HPGe gamma spectroscopy. Log D_{7.4} was calculated according to Equation S3.

$$log {D}_{7.4} = {log}_{10} ( \frac{Activity in Octanol}{Activity in PBS} ) \qquad ( S3 )$$

### 1.8 In Vivo Studies

### 1.8.1 Mouse Imaging and PET Vs SPECT Imaging Characterization

Mouse imaging and biodistribution studies were conducted according to IACUC approved animal protocols.

For mouse imaging, PET vs. SPECT imaging characterization, in vivo stability, and biodistribution measurements, ¹¹⁷Sb was prepared with two different solvent-extraction methods. To measure biodistribution of unchelated ¹¹⁷Sb, prepare ¹¹⁷Sb for Derenzo phantom filling, and provide a control for in vivo stability measurements, back-extraction directly into PBS aqueous phase was used, likely resulting in [¹¹⁷Sb]Sb(OH)₆⁻. (Filella, M.; Belzile, N.; Chen, Y. W. Antimony in the Environment: A Review Focused on Natural Waters II. Relevant Solution Chemistry. Earth-Sci. Rev. 2002, 59 (1-4), 265-285.) After separation of the two phases, any residual DBE was removed by heating to 50° C. and blowing argon over the sample. For preparation of [¹¹⁷Sb]Sb-TREN-CAM, radioantimony was back-extracted into 0.5 M NH₄OAc (pH 4). When radiolabeling, 2 mL ^{1XX}Sb in NH₄OAc solution was combined with 220 μL of TREN-CAM (as a 10 mM solution in 20% DMSO/H₂O) and heated to 80° C. for 1 h. Quantitative radiolabeling was confirmed via normal-phase TLC (Si plates with MeOH mobile phase). A 1:5 dilution of radiolabeling solution was passed through a preconditioned (5 mL EtOH, 5 mL H₂O) C8 Sep-Pak® cartridge (Waters, Milford, MA, USA), trapping [¹¹⁷Sb]Sb-TREN-CAM. The cartridge was rinsed with 1 mL H₂O, and [¹¹⁷Sb]Sb-TREN-CAM was eluted with EtOH, collecting dropwise. Purified [¹¹⁷Sb]Sb-TREN-CAM was diluted with PBS (800 μL), and the EtOH was evaporated from the solution using a gentle N₂ flow. For imaging studies, 46.5 MBq±0.1% (1.3 mCi) [¹¹⁷Sb]Sb-TREN-CAM was produced and purified at a molar activity of 1.1 μCi/μmol TREN-CAM (uncertainty is expressed as statistical counting uncertainty). For comparison with unchelated ^{1XX}Sb, the studies used 62.5 MBq±0.1% (1.7 mCi) of [¹¹⁷Sb]Sb(OH)₆⁻ produced in PBS.

Two groups of healthy 3-month-old male BALB/c mice (N=3) (Jackson River Laboratories) were injected with 150 μL ¹¹⁷Sb solutions. The control, unchelated Sb group received 12.51 MBq±0.04 MBq (338.0 μCi±1.0 μCi) ¹¹⁷Sb, and the [¹¹⁷Sb]Sb-TREN-CAM group received 8.22 MBq±0.02 MBq (222.2 μCi±0.5 μCi) injected ¹¹⁷Sb activity. Thirty-minute SPECT/CT images were collected using a MILabs U-SPECT/CTUHR at 20, 90, and 240 min timepoints post injection. For SPECT imaging, all 3 mice were imaged simultaneously in the prone position under isoflourine anesthesia (3% induction, 1.5% maintenance). SPECT image reconstructions windowed both ¹¹⁷Sb (159 keV) and ¹¹⁹Sb (˜25 keV) emissions. Twenty-minute PET/CT images (time window 3.432 ns; energy window 350-650 keV) of two mice in the prone position were collected 150 min post injection using a Siemens Inveon micro-PET/CT under isoflourine anesthesia as noted above. In both cases, the scanner was calibrated by imaging vials of ¹¹⁷Sb activity and calibrating image-derived volumetric activity concentrations to activity measured via High Purity Germanium (HPGe) gamma spectroscopy. CT images collected after each SPECT or PET image were used to attenuation-correct and anatomically co-register datasets. 3-D ordered subset expectation maximization/maximum a posteriori (OSEM3D/MAP) without scatter correction was used to reconstruct CT attenuation-corrected PET images. SPECT images (energy window 147.1-170.9 keV) were reconstructed with attenuation-correction and a pixel-based, accelerated similarity-regulated ordered subsets expectation maximization (SROSEM) algorithm with 128 subsets, 5 iterations, 0.4 mm voxel size, and 1.4 mm gaussian blurring. Using co-registered CT images, manual volume-of-interest delineation of tissues allowed activity biodistribution measurement, as expressed as percent injected activity per gram of tissue (% IA/g) using either Inveon Research Workstation (Siemens) for PET VOI analysis or Imalytics 3.0 (MiLabs) for SPECT VOI analysis.

For ¹¹⁷Sb PET and SPECT characterization and comparison, a linear channel Derenzo D271020 phantom (Phantech, Madison, WI) with rod sizes O 1.0, 1.2, 1.4, 1.6, 1.8, 2.0 mm filled with ˜5.5 MBq (˜150 uCi) ¹¹⁷Sb was imaged using the aforementioned PET and SPECT imaging conditions. After image reconstruction, central axial slices were exported, and line profiles of rod signal intensity created using the open-source software ImageJ (NIH) to draw lines intersecting the center of the central most rod with the center of the outermost rod for each rod size cluster. The spatial resolutions of the systems were expressed as full width half max (FWHM) of the central most rod with contrasts (C_{rod}) of the systems calculated according to Equation 2, where Rₘₐₓ is the maximum and Rₘᵢₙ the minimum rod signal intensity values determined from the line profiles.

$${C}_{rod} = ( \frac{{R}_{max} - {R}_{min}}{{R}_{max} + {R}_{min}} )$$

### 1.8.2 Ex Vivo Biodistribution

5 h post injection, animals were sacrificed using CO₂ asphyxiation with cardiac exsanguination and secondary euthanasia confirmation. Tissues and organs were harvested and weighed, and the ¹¹⁷Sb activity within these tissues was counted using a PerkinElmer Gamma Counter (Waltham, MA, USA) for ex vivo biodistribution measurements.

### 1.8.3 Ex Vivo Metabolite Analysis

Three healthy 3-month-old male BALB/c mice (Jackson River Laboratories) were injected with 0.47 MBq±0.17 MBq (12.7 μCi±4.5 μCi) [¹¹⁷Sb]Sb-TREN-CAM in 150 μL PBS. Here, the uncertainty is expressed as the standard deviation of replicates. Thirty minutes post injection, animals were sacrificed, and intact gallbladders were harvested. Gallbladders were lanced, and their contents washed with MeOH (50 μL). Aliquots of the supernatant (5 μL) were spotted on Al-backed Si TLC plates next to triplicate [¹¹⁷Sb]Sb-TREN-CAM controls. The TLC plates were developed using a MeOH mobile phase. Under these conditions, TREN-CAM-complexed radio-Sb migrates near the solvent front, whereas unchelated radio-Sb remains near the baseline. Radio-TLC visualization with a Packard Cyclone phosphor plate reader allowed activity distribution and measurement of R_{f} for both metabolized [¹¹⁷Sb]Sb-TREN-CAM and controls.

### Example 2

This Example illustrates the synthesis of bifunctional derivatives of TREN-CAM and the conjugation of biological targeting vectors using the bifunctional derivatives.

### Examples of Bifunctional Derivatives of TREN-CAM:

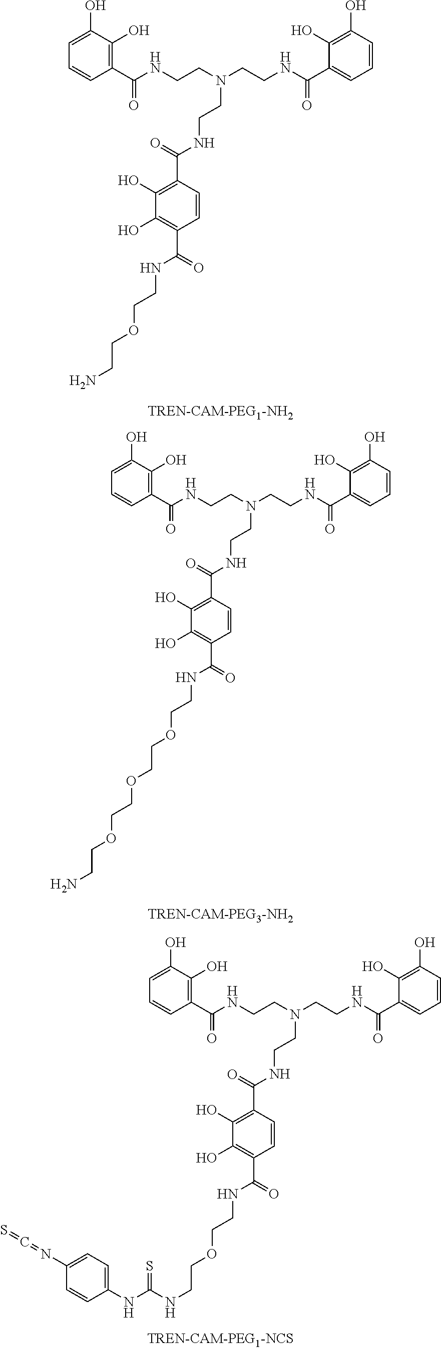 [SMILES: NCCOCCNC(=O)c1ccc(C(=O)NCCN(CCNC(=O)c2cccc(O)c2O)CCNC(=O)c2cccc(O)c2O)c(O)c1O ; NCCOCCOCCOCCNC(=O)c1ccc(C(=O)NCCN(CCNC(=O)c2cccc(O)c2O)CCNC(=O)c2cccc(O)c2O)c(O)c1O ; O=C(NCCN(CCNC(=O)c1cccc(O)c1O)CCNC(=O)c1ccc(C(=O)NCCOCCNC(=S)Nc2ccc(N=C=S)cc2)c(O)c1O)c1cccc(O)c1O]

### Examples of Targeted Conjugates of TREN-CAM:

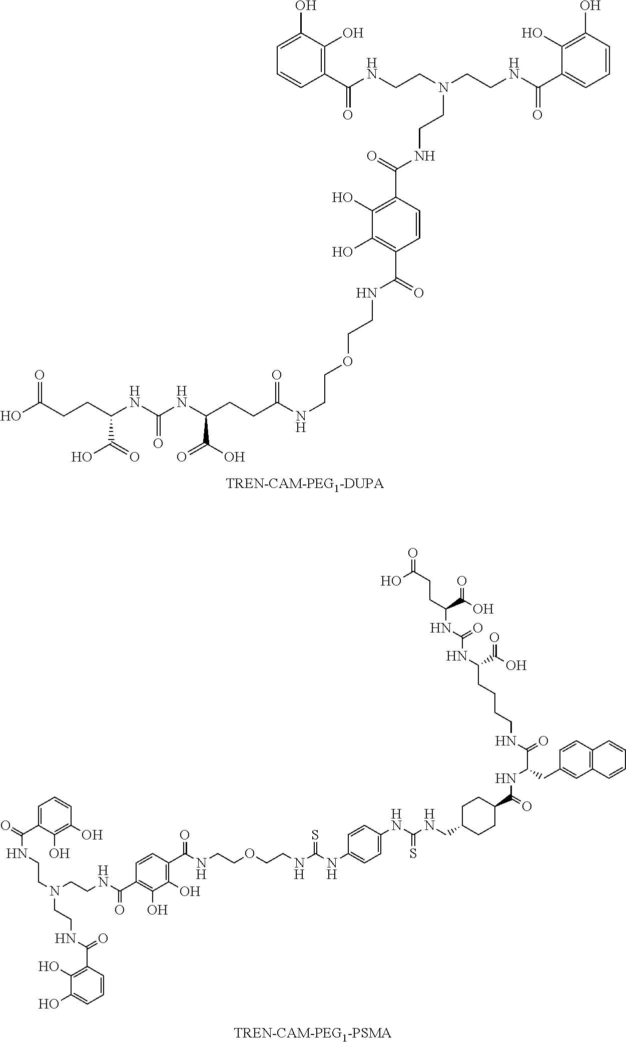 [SMILES: O=C(O)CC[C@H](NC(=O)N[C@@H](CCC(=O)NCCOCCNC(=O)c1ccc(C(=O)NCCN(CCNC(=O)c2cccc(O)c2O)CCNC(=O)c2cccc(O)c2O)c(O)c1O)C(=O)O)C(=O)O ; O=C(O)CC[C@H](NC(=O)N[C@@H](CCCCNC(=O)[C@H](Cc1ccc2ccccc2c1)NC(=O)[C@H]1CC[C@H](CNC(=S)Nc2ccc(NC(=S)NCCOCCNC(=O)c3ccc(C(=O)NCCN(CCNC(=O)c4cccc(O)c4O)CCNC(=O)c4cccc(O)c4O)c(O)c3O)cc2)CC1)C(=O)O)C(=O)O]

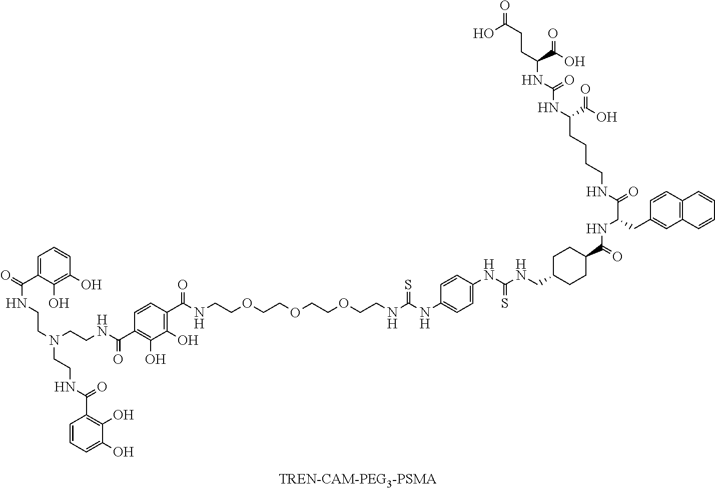 [SMILES: O=C(O)CC[C@H](NC(=O)N[C@@H](CCCCNC(=O)[C@H](Cc1ccc2ccccc2c1)NC(=O)[C@H]1CC[C@H](CNC(=S)Nc2ccc(NC(=S)NCCOCCOCCOCCNC(=O)c3ccc(C(=O)NCCN(CCNC(=O)c4cccc(O)c4O)CCNC(=O)c4cccc(O)c4O)c(O)c3O)cc2)CC1)C(=O)O)C(=O)O]

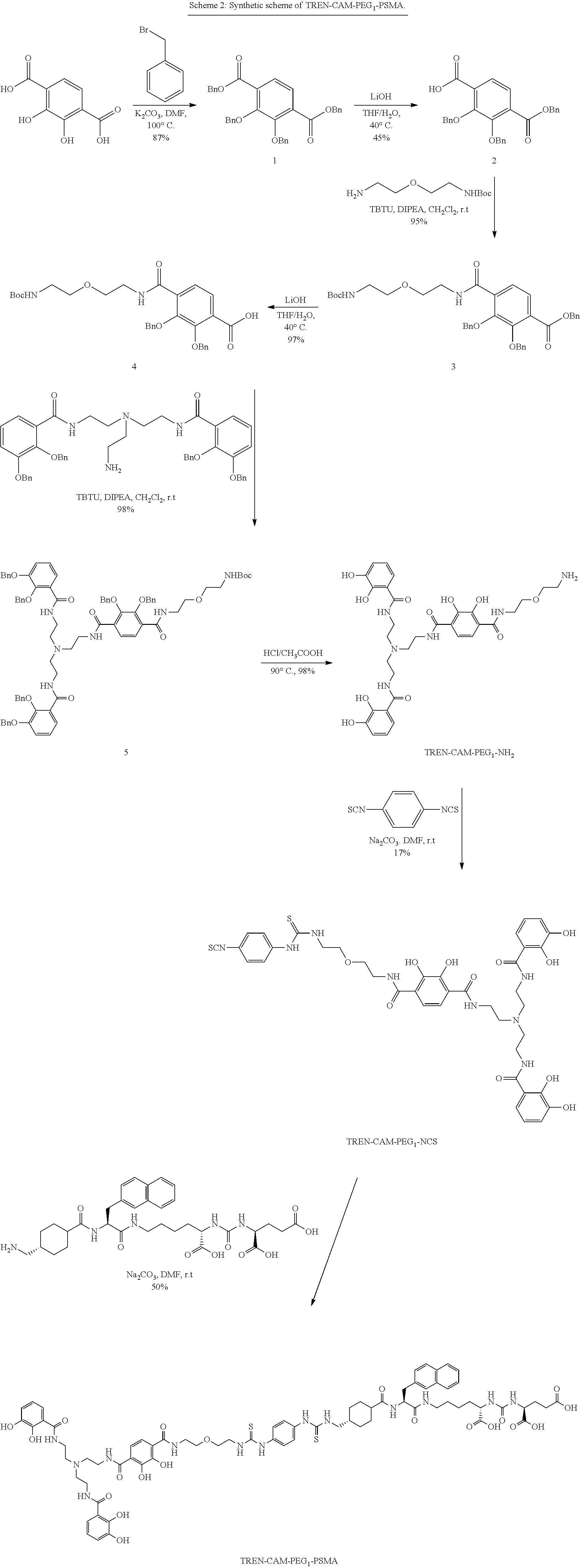

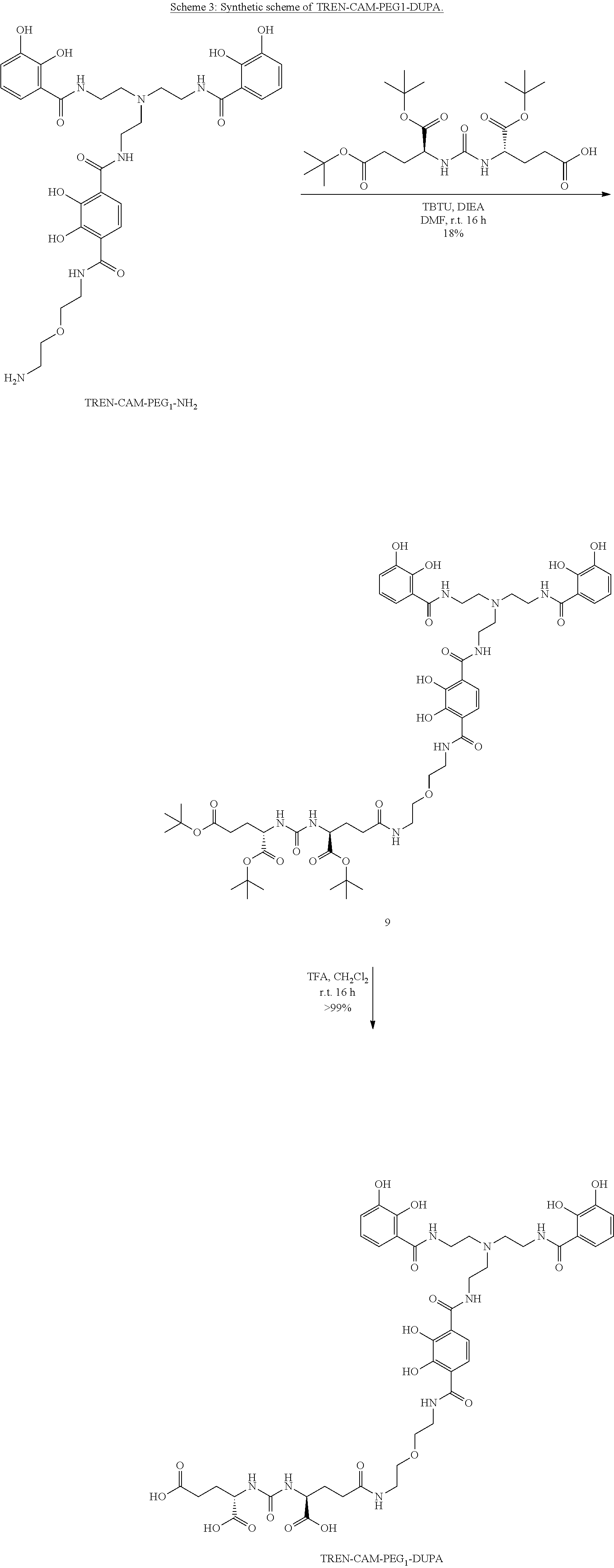 [SMILES: NCCOCCNC(=O)c1ccc(C(=O)NCCN(CCNC(=O)c2cccc(O)c2O)CCNC(=O)c2cccc(O)c2O)c(O)c1O ; CC(C)(C)OC(=O)CC[C@H](NC(=O)N[C@@H](CCC(=O)O)C(=O)OC(C)(C)C)C(=O)OC(C)(C)C ; CC(C)(C)OC(=O)CC[C@H](NC(=O)N[C@@H](CCC(=O)NCCOCCNC(=O)c1ccc(C(=O)NCCN(CCNC(=O)c2cccc(O)c2O)CCNC(=O)c2cccc(O)c2O)c(O)c1O)C(=O)OC(C)(C)C)C(=O)OC(C)(C)C ; O=C(O)CC[C@H](NC(=O)N[C@@H](CCC(=O)NCCOCCNC(=O)c1ccc(C(=O)NCCN(CCNC(=O)c2cccc(O)c2O)CCNC(=O)c2cccc(O)c2O)c(O)c1O)C(=O)O)C(=O)O]

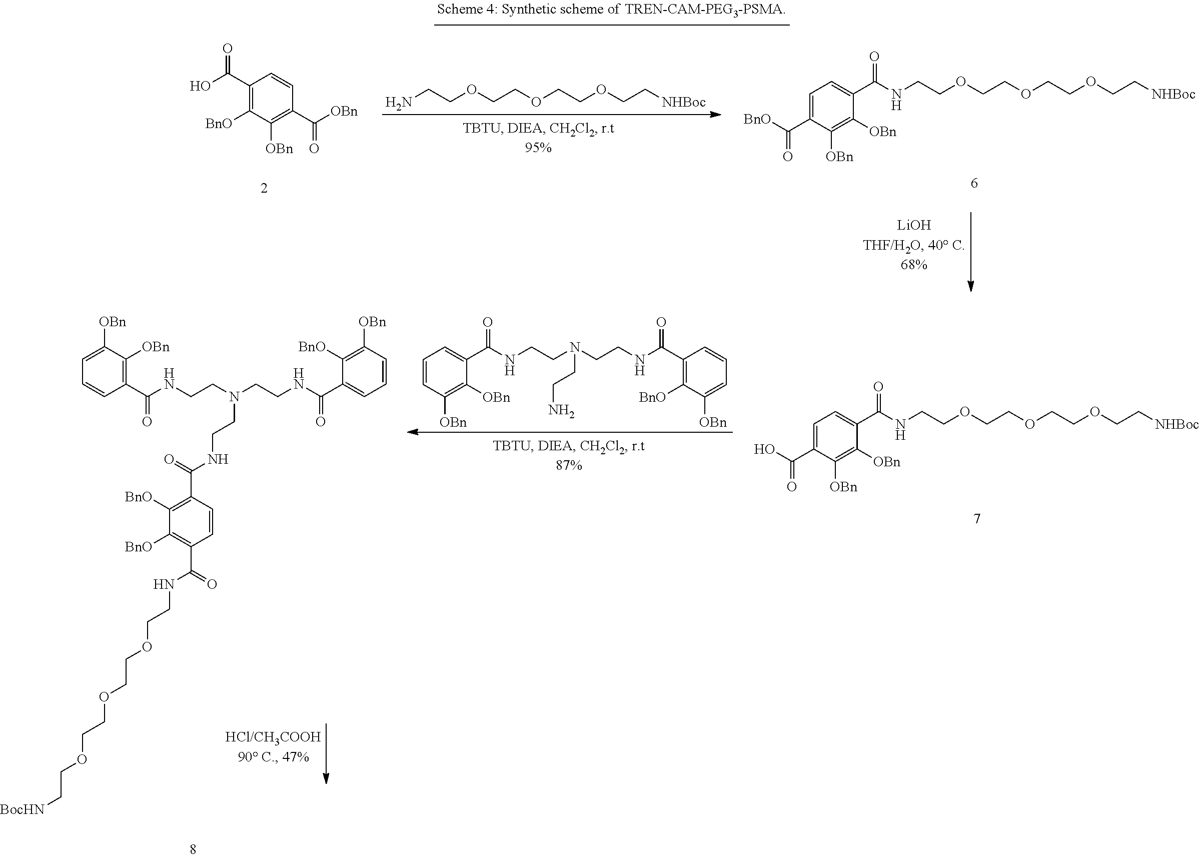 [SMILES: O=C(O)c1ccc(C(=O)OCc2ccccc2)c(OCc2ccccc2)c1OCc1ccccc1 ; CC(C)(C)OC(=O)NCCOCCOCCOCCN ; CC(C)(C)OC(=O)NCCOCCOCCOCCNC(=O)c1ccc(C(=O)OCc2ccccc2)c(OCc2ccccc2)c1OCc1ccccc1 ; CC(C)(C)OC(=O)NCCOCCOCCOCCNC(=O)c1ccc(C(=O)NCCN(CCNC(=O)c2cccc(OCc3ccccc3)c2OCc2ccccc2)CCNC(=O)c2cccc(OCc3ccccc3)c2OCc2ccccc2)c(OCc2ccccc2)c1OCc1ccccc1 ; NCCN(CCNC(=O)c1cccc(OCc2ccccc2)c1OCc1ccccc1)CCNC(=O)c1cccc(OCc2ccccc2)c1OCc1ccccc1 ; CC(C)(C)OC(=O)NCCOCCOCCOCCNC(=O)c1ccc(C(=O)O)c(OCc2ccccc2)c1OCc1ccccc1]

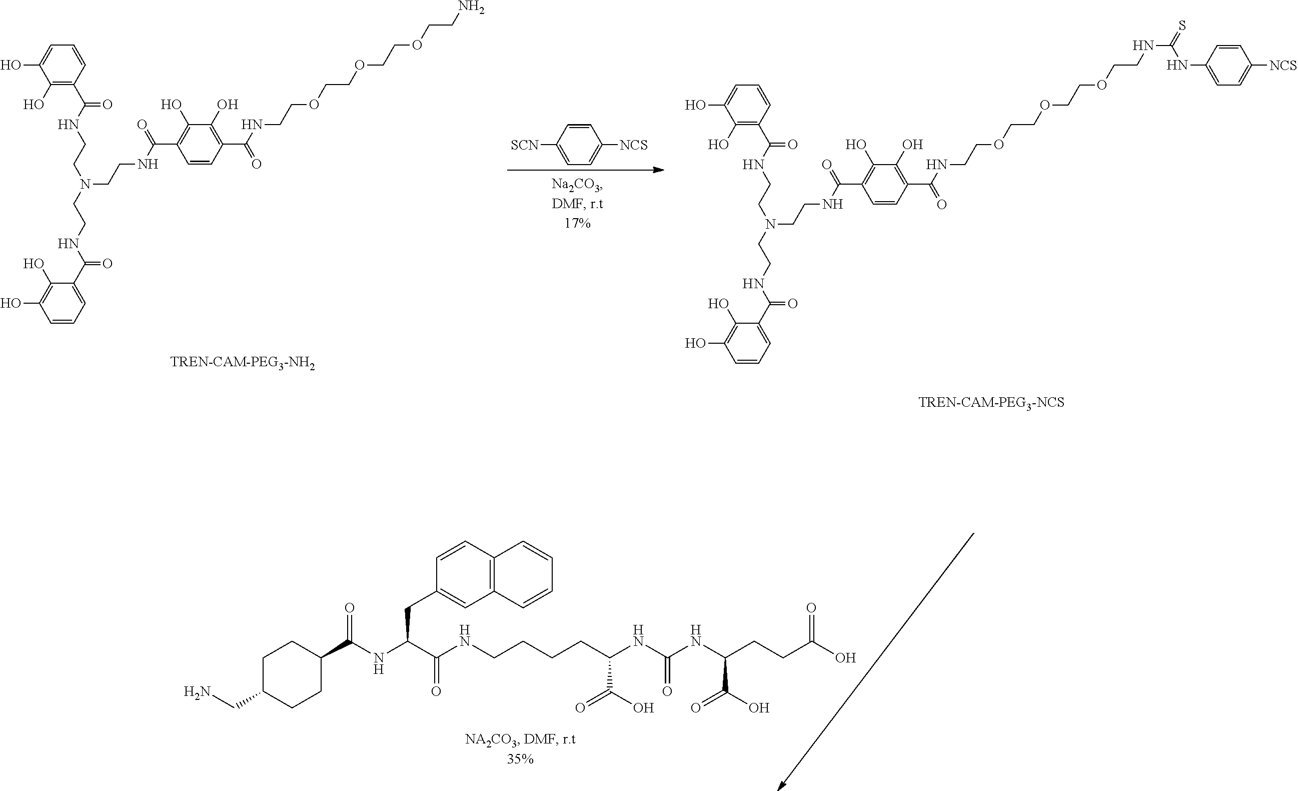

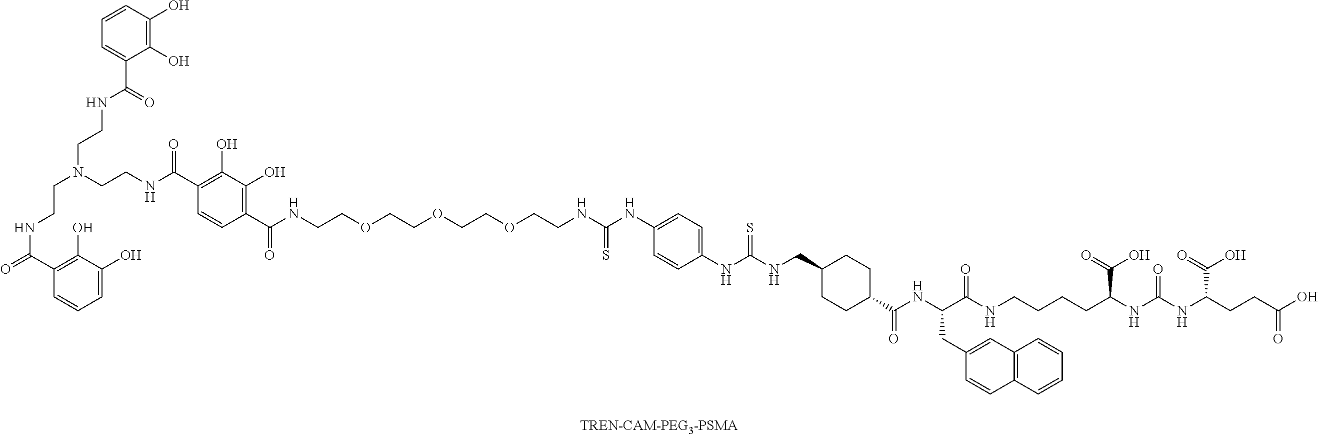 [SMILES: O=C(O)CC[C@H](NC(=O)N[C@@H](CCCCNC(=O)[C@H](Cc1ccc2ccccc2c1)NC(=O)[C@H]1CC[C@H](CNC(=S)Nc2ccc(NC(=S)NCCOCCOCCOCCNC(=O)c3ccc(C(=O)NCCN(CCNC(=O)c4cccc(O)c4O)CCNC(=O)c4cccc(O)c4O)c(O)c3O)cc2)CC1)C(=O)O)C(=O)O]

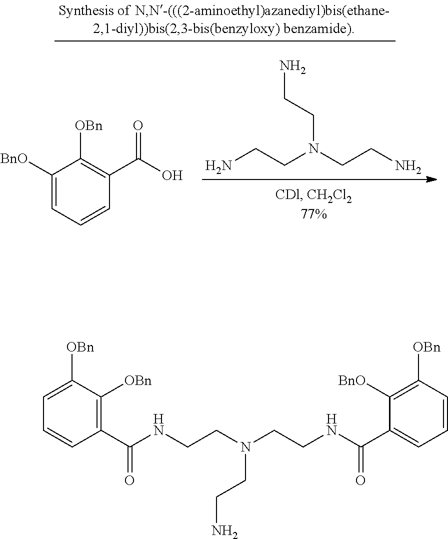 [SMILES: O=C(O)c1cccc(OCc2ccccc2)c1OCc1ccccc1 ; NCCN(CCN)CCN ; NCCN(CCNC(=O)c1cccc(OCc2ccccc2)c1OCc1ccccc1)CCNC(=O)c1cccc(OCc2ccccc2)c1OCc1ccccc1]

2,3-bis(benzyloxy)benzoic acid (8.17 g, 24.4 mmol) and CDI (4.00 g, 24.7 mmol) were refluxed in CH₂Cl₂ (250 mL) for 2 h. The mixture was allowed to cool to r.t. and then slowly added to a solution of tris(2-aminoethyl)amine (TREN, 1.83 mL, 12.2 mmol) in CH₂Cl₂ (100 mL) over the course of 3 h. The resulting mixture was allowed to stir at r.t. overnight. Upon completion, the reaction mixture was washed with 4 M NaOH (aq). The organic layer was concentrated, and the product was isolated using an automated column (220 g Gold prepacked silica, 0%-20% MeOH in CH₂Cl₂ gradient) to yield a pale yellow solid (7.32 g, 77%). ¹H NMR (400 MHz, CDCl₃) δ 8.26 (br s, 2H), 7.98 (t, J=5.9 Hz, 2H), 7.48-7.27 (m, 22H), 7.05 (dd, J=8.2, 1.8 Hz, 2H), 6.99 (t, J=7.9 Hz, 2H), 5.10 (s, 4H), 5.05 (s, 4H), 3.19 (q, J=6.1 Hz, 4H), 2.69 (m, 4H), 2.31 (t, J=6.1 Hz, 4H). ¹³C NMR (101 MHz, CDCl₃) δ 166.19, 151.61, 146.46, 136.54, 136.43, 128.87, 128.78, 128.68, 128.24, 127.75, 127.65, 124.46, 122.72, 116.83, 76.49, 71.22, 54.46, 51.71, 38.31, 37.82.

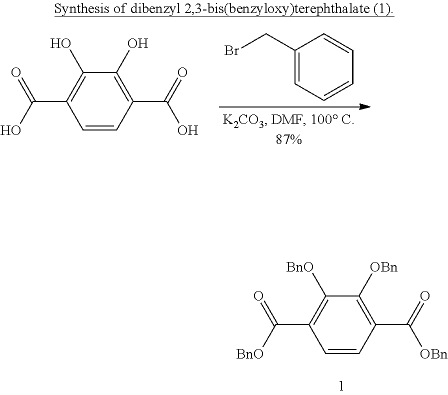 [SMILES: O=C(O)c1ccc(C(=O)O)c(O)c1O ; BrCc1ccccc1 ; O=C(OCc1ccccc1)c1ccc(C(=O)OCc2ccccc2)c(OCc2ccccc2)c1OCc1ccccc1]

In a 1 L round-bottom flask, 2,3-dihydroxyterephthalic acid (7.00 g, 35.33 mmol) was dissolved in dry DMF (300 mL). K₂CO₃ (48.82 g, 353.3 mmol) was added, and the mixture was stirred at 100° C. under N₂ for 3 h. Then, a solution of benzyl bromide (33.5 mL, 282.64 mmol) in dry DMF (50 mL) was added, and the reaction mixture was allowed to stir at 100° C. under nitrogen for 2 days. The mixture was then filtered, and the filtrate was concentrated under rotary evaporation. The product was isolated using an automated column (80 g normal phase prepacked silica, 0%-10% MeOH in CH₂Cl₂ gradient) to yield an amber oil (12.31 g, 87%). ¹H NMR (400 MHz, CDCl₃) δ 7.59 (s, 2H), 7.45-7.29 (m, 20H), 5.34 (s, 4H), 5.12 (s, 4H). ¹³C NMR (101 MHz, CDCl₃) δ 165.04, 152.86, 136.62, 135.52, 130.31, 128.67, 128.58, 128.44, 128.36, 128.31, 128.11, 125.65, 76.35, 67.20.

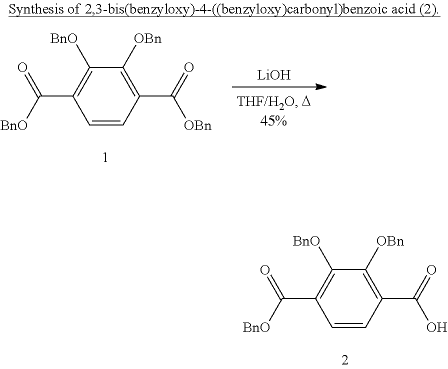 [SMILES: O=C(OCc1ccccc1)c1ccc(C(=O)OCc2ccccc2)c(OCc2ccccc2)c1OCc1ccccc1 ; "LiOH" ; O=C(O)c1ccc(C(=O)OCc2ccccc2)c(OCc2ccccc2)c1OCc1ccccc1]

Dibenzyl 2,3-bis(benzyloxy)terephthalate (8.00 g, 14.32 mmol) was added to a 300 mL round-bottom flask and dissolved in THF (100 mL). LiOH (343 mg, 14.32 mmol) was dissolved in 5 mL H₂O and added to the THF solution. The reaction was stirred at 40° C. overnight, then refluxed for 1 h the following day. Afterwards, the reaction mixture was cooled to r.t., neutralized with HCl (1 M), and extracted into DCM (3×50 mL). The organic layers were concentrated under rotary evaporation. The product was isolated using an automated column (80 g prepacked silica, 0%-10% MeOH in CH₂Cl₂ gradient) (3.02 g, 45%). ¹H NMR (400 MHz, CDCl₃) δ 10.96 (s, 1H), 7.91 (d, J=8.4 Hz, 1H), 7.66 (d, J=8.3 Hz, 1H), 7.44-7.27 (m, 15H), 5.35 (s, 2H), 5.29 (s, 2H), 5.11 (s, 2H).

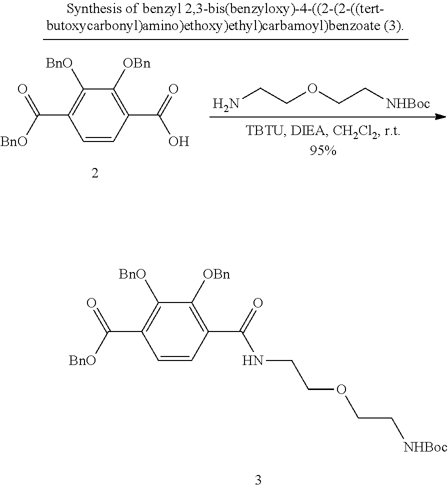 [SMILES: O=C(O)c1ccc(C(=O)OCc2ccccc2)c(OCc2ccccc2)c1OCc1ccccc1 ; CC(C)(C)OC(=O)NCCOCCN ; CC(C)(C)OC(=O)NCCOCCNC(=O)c1ccc(C(=O)OCc2ccccc2)c(OCc2ccccc2)c1OCc1ccccc1]

In a 250 mL round-bottom flask, 2,3-bis(benzyloxy)-4-((benzyloxy)carbonyl)benzoic acid (569 mg, 1.21 mmol) was dissolved in dry CH₂Cl₂ followed by addition of DIEA (232 mL, 1.34 mmol). Tert-butyl (2-(2-aminoethoxy)ethyl)carbamate (273 mg, 1.34 mmol) was then dissolved in dry CH₂Cl₂ (10 mL) and added to the previous solution followed by addition of TBTU (467 mg, 1.46 mmol). The mixture was allowed to stir at r.t. under N₂ overnight. Upon completion, the reaction mixture was concentrated under rotary evaporation and the product was isolated using an automated column (40 g prepacked silica, 0%-10% MeOH in CH₂Cl₂ gradient) (753 mg, 95%). ¹H NMR (400 MHz, CDCl₃) δ 8.13 (t, J=5.2 Hz, 1H), 7.92 (d, J=8.3 Hz, 1H), 7.65 (d, J=8.3 Hz, 1H), 7.44-7.28 (m, 15H), 5.33 (s, 2H), 5.12 (d, J=2.7 Hz, 4H), 4.78 (s, 1H), 3.52-3.48 (m, 2H), 3.44 (t, J=5.0 Hz, 2H), 3.37 (t, J=5.2 Hz, 2H), 3.17 (d, J=5.5 Hz, 2H), 1.40 (s, 9H).

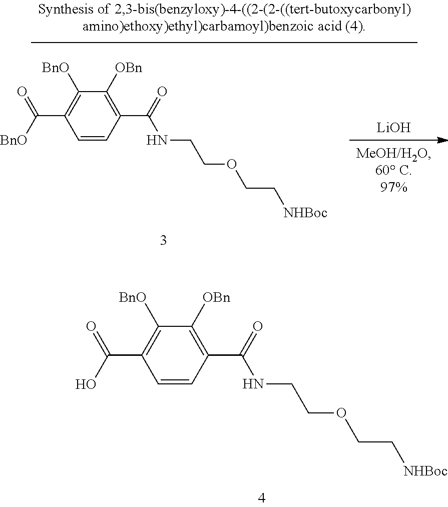

In a 250 mL round-bottom flask, benzyl 2,3-bis(benzyloxy)-4-((2-(2-((tert-butoxycarbonyl)amino)ethoxy)ethyl)carbamoyl)benzoate (600 mg, 0.916 mmol) was dissolved in THF (30 mL). Then, LiOH (219 mg, 9.16 mmol) was dissolved in H₂O (5 mL) and added to the THF solution. The reaction mixture was allowed to stir at 60° C. overnight. Upon completion, the reaction mixture was cooled to r.t., neutralized with HCl (1 M), and the product was extracted into CH₂Cl₂ (3×30 mL). The organic layers were dried with MgSO₄, filtered, and concentrated under rotary evaporation. The product was isolated using an automated column (24 g Gold prepacked silica, 0%-5% MeOH in CH₂Cl₂ gradient) (501 mg, 97%). ¹H NMR (400 MHz, CDCl₃) δ 8.13 (t, J=5.2 Hz, 1H), 7.92 (d, J=8.3 Hz, 1H), 7.65 (d, J=8.3 Hz, 1H), 7.44-7.28 (m, 15H), 5.33 (s, 2H), 5.12 (d, J=2.7 Hz, 4H), 4.78 (s, 1H), 3.52-3.48 (m, 2H), 3.44 (t, J=5.0 Hz, 2H), 3.37 (t, J=5.2 Hz, 2H), 3.17 (d, J=5.5 Hz, 2H), 1.40 (s, 9H).

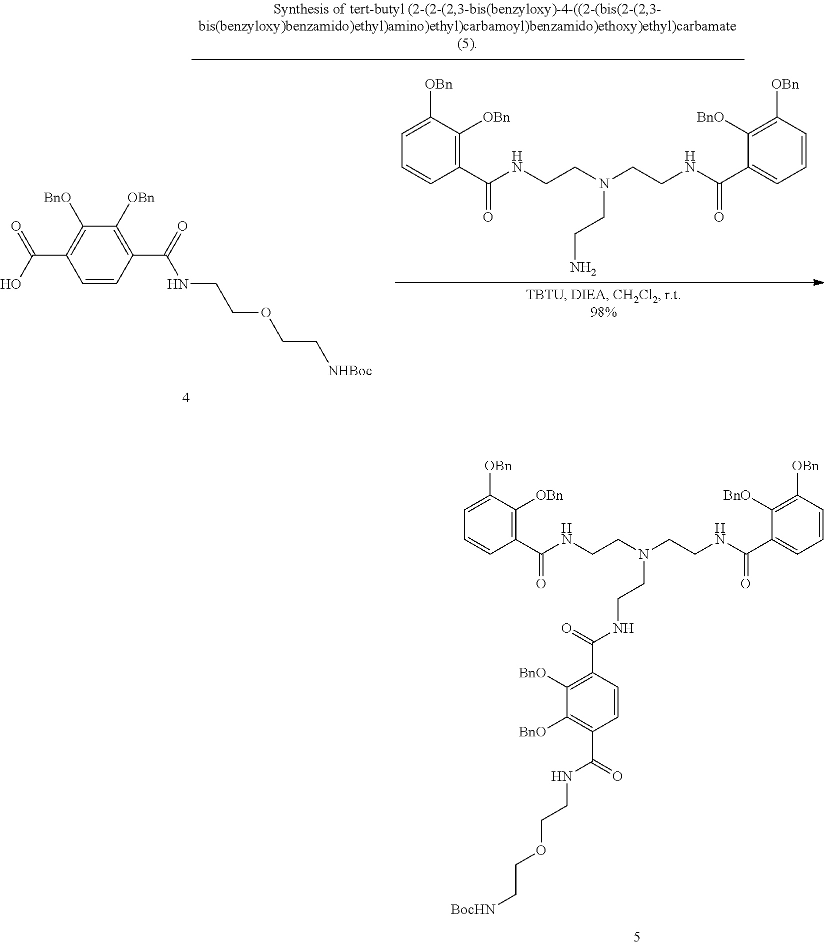

In a 250 mL round-bottom flask, N,N′-(((2-aminoethyl)azanediyl)bis(ethane-2,1-diyl))bis(2,3-bis(benzyloxy)benzamide) (501 mg, 0.644 mmol) was dissolved in dry CH₂Cl₂ (30 mL), followed by addition of DIEA (0.133 mL, 0.773 mmol). Then, 2,3-bis(benzyloxy)-4-((2-(2-((tert-butoxycarbonyl)amino)ethoxy)ethyl)carbamoyl)benzoic acid (400 mg, 0.708 mmol) was dissolved in dry CH₂Cl₂ (20 mL) and added to the previous solution followed by addition of TBTU (227 mg, 0.708 mmol). The mixture was allowed to stir at r.t. under N₂ overnight. Upon completion, the reaction mixture was concentrated under rotary evaporation and the product was isolated using an automated column (40 g Gold prepacked silica, 0%-10% MeOH in CH₂Cl₂) (890 mg, 98%). ¹H NMR (400 MHz, DMSO-d₆) δ 8.46-8.60 (m, 1H), 8.35 (s, 1H), 8.24-8.09 (m, 2H), 7.49 (d, J=8.4 Hz, 4H), 7.43-7.21 (m, 31H), 7.16-7.04 (m, 4H), 6.77 (d, J=5.1 Hz, 1H), 5.19 (d, J=9.8 Hz, 4H), 4.96-5.07 (m, 8H), 3.58 (s, 2H), 3.49-3.35 (m, 8H), 3.12-3.32 (m, 8H), 3.04 (q, J=5.9 Hz, 2H), 1.34 (s, 9H).

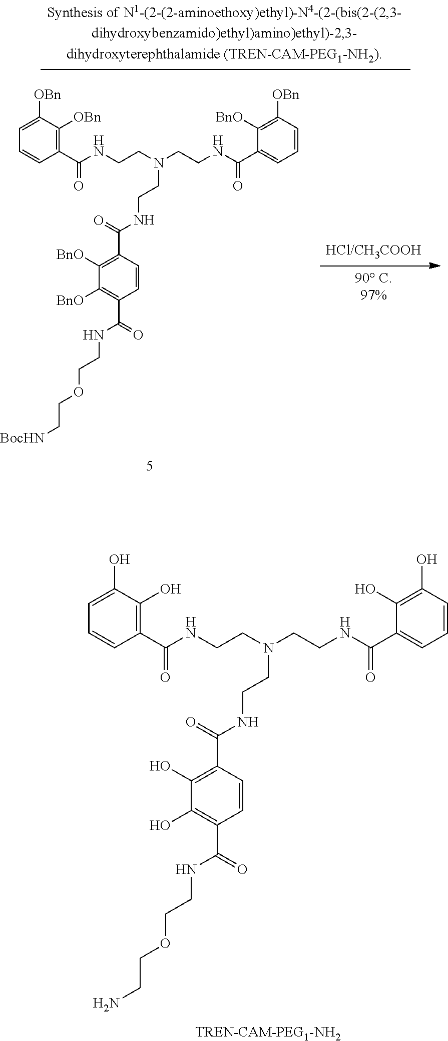 [SMILES: CC(C)(C)OC(=O)NCCOCCNC(=O)c1ccc(C(=O)NCCN(CCNC(=O)c2cccc(OCc3ccccc3)c2OCc2ccccc2)CCNC(=O)c2cccc(OCc3ccccc3)c2OCc2ccccc2)c(OCc2ccccc2)c1OCc1ccccc1 ; "90° C.97%" ; "HCl/CH3COOH" ; NCCOCCNC(=O)c1ccc(C(=O)NCCN(CCNC(=O)c2cccc(O)c2O)CCNC(=O)c2cccc(O)c2O)c(O)c1O]

In a 50 mL round-bottom flask, tert-butyl (2-(2-(2,3-bis(benzyloxy)-4-((2-(bis(2-(2,3-bis(benzyloxy)benzamido)ethyl)amino)ethyl)carbamoyl)benzamido)ethoxy)ethyl)carbamate (500 mg, 0.377 mmol) was dissolved in glacial CH₃COOH (10 mL). Concentrated HCl (aq, 1 mL) was then added, and the reaction mixture was allowed to stir at 90° C. The reaction was monitored by HPLC and had gone to completion after 8 h. Upon completion, the reaction mixture was concentrated under rotary evaporation and purified using preparatory HPLC to isolate the title compound (TREN-CAM-PEG₁-NH₂, 250 mg, 97%). ¹H NMR (400 MHz, DMSO) δ 12.69 (s, 1H), 12.21 (s, 1H), 12.10 (s, 2H), 9.86 (s, 1H), 9.35 (s, 1H), 9.09 (s, 1H), 8.95 (s, 3H), 7.83 (s, 3H), 7.37 (d, J=8.8 Hz, 1H), 7.30 (d, J=8.8 Hz, 1H), 7.23 (dd, J=8.2, 1.6 Hz, 2H), 6.94 (dd, J=7.8, 1.5 Hz, 2H), 6.69 (t, J=8.0 Hz, 2H), 3.70 (s, 5H), 3.61 (q, J=5.1 Hz, 5H), 3.57-3.43 (m, 6H), 2.99 (p, J=5.6 Hz, 2H).

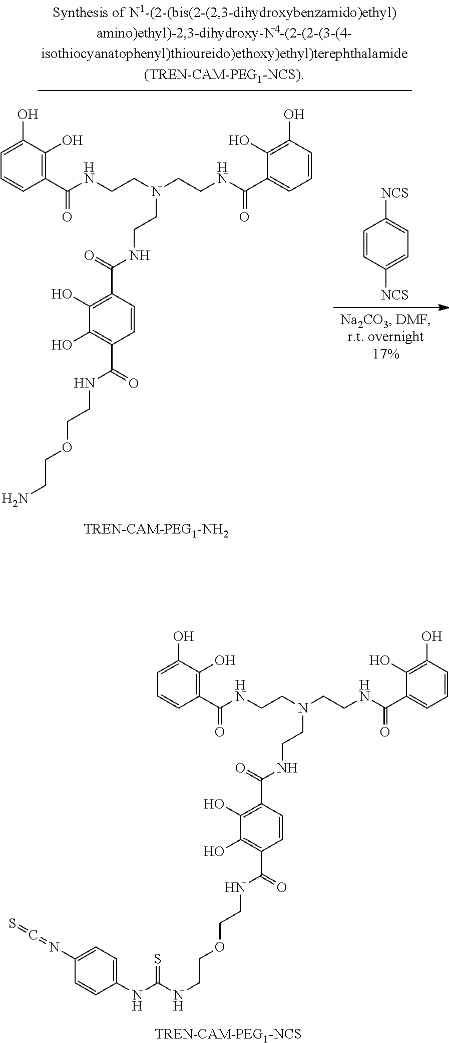

In a 20 mL vial, N1-(2-(2-aminoethoxy)ethyl)-N4-(2-(bis(2-(2,3-dihydroxybenzamido)ethyl)amino)ethyl)-2,3-dihydroxyterephthalamide (42 mg, 61.3 μmol) was dissolved in dry DMF (2 mL) followed by addition of Na₂CO₃ (65 mg, 613 μmol). The suspension was allowed to stir at r.t. under Ar for 30 min. Afterwards, p-phenylene diisothiocyanate (118 mg, 613 μmol) was added and the mixture was allowed to stir at r.t. sealed under Ar overnight. Upon completion, the reaction mixture was centrifuged, and the supernatant was collected. The residual solid was resuspended in DMF (2 mL). The mixture was centrifuged, and the supernatants were combined. Diethyl ether (60 mL) was added to the combined supernatant, and this mixture was placed in the freezer overnight. Afterward, the solution was centrifuged, the supernatant was decanted, and the remaining solid was dried under reduced pressure. The resulting residue was dissolved in MeCN:H₂O (1:1, 700 μL) and the product was isolated using preparatory HPLC (5 μm C18 column, 10% MeCN(aq)-100% MeCN gradient over 40 min with a constant TFA concentration of 0.1%) to yield a white solid (TREN-CAM-PEG₁-NCS, 9 mg, 86.8% pure, 17%). ESI-MS: Measured: m/z 877.2631. Calcd for [C₄₀H₄₅N₈O₁₁S₂]⁺: m/z 877.2644.

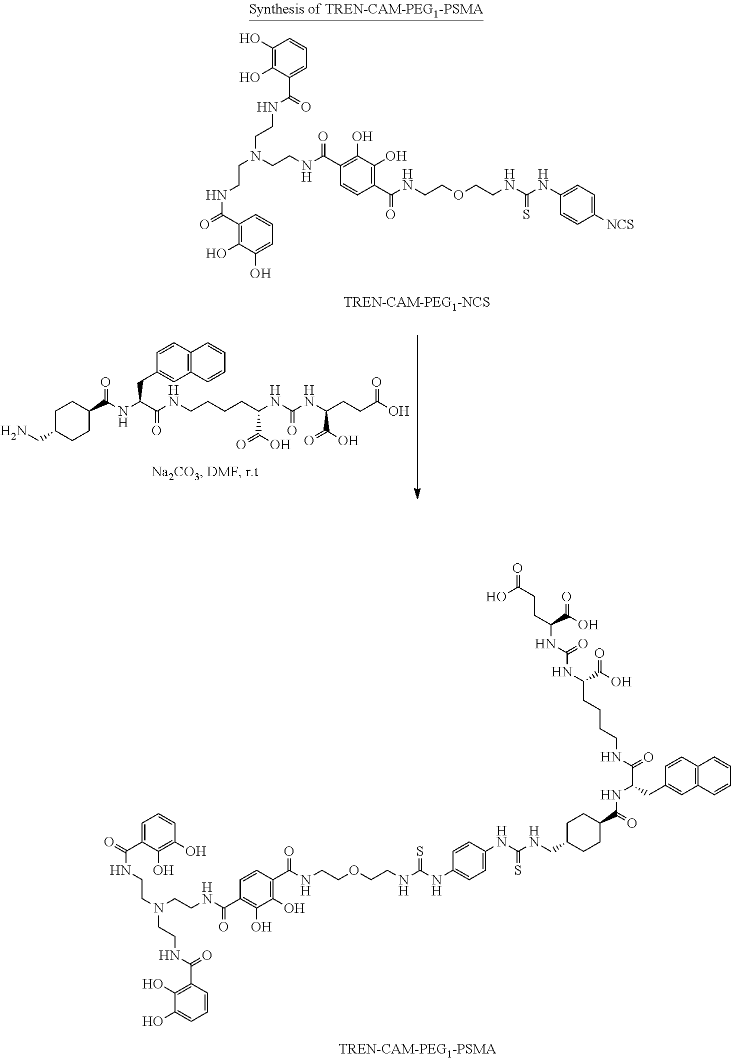

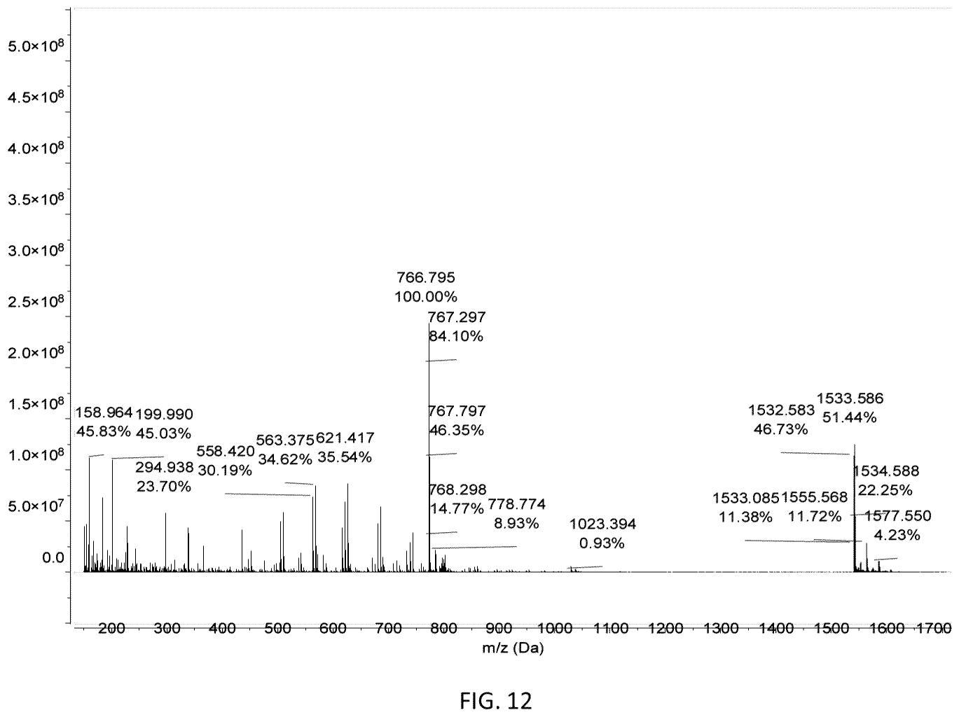

In a 20 mL vial, N1-(2-(bis(2-(2,3-dihydroxybenzamido)ethyl)amino)ethyl)-2,3-dihydroxy-N4-(2-(2-(3-(4-isothiocyanatophenyl)thioureido)ethoxy)ethyl)terephthalamide (TREN-CAM PEG₁-NCS, 12 mg, 13.68 μmol) was dissolved in 2 mL of anhydrous DMF. Then, the solution was basified with Na₂CO₃ (14.41 mg, 136.8 μmol, 10 equiv). The suspension was stirred vigorously for 10 min at RT before addition of vipivotide tetraxetan ligand-linker conjugate (PSMA-617 ligand-linker conjugate, MedChemExpress, 8.89 mg, 13.6 μmol, 1 eq). The resulting mixture was stirred in the dark overnight. Upon completion, the reaction mixture was separated via centrifugation and the insoluble salts washed with DMF (2×3 mL). The combined supernatant was diluted with diethyl ether (50 mL) and kept at −20° C. for 2 h to induce precipitation. The crude product was isolated via centrifugation and washed with diethyl ether (2×20 mL). The crude material was then purified via preparative HPLC (A: H₂O (0.1% TFA), B: MeCN (0.1% TFA); 0-5 min: 90% A, 10% B, 5-45 min: 90% A to 100% B, 45-55 min: 100% B, 14 mL/min). Appropriate product fractions were pooled and lyophilized to give the title compound as a white solid (8.8 mg, 50%). ESI-MS: Measured: m/z 1532.5865. Calcd for [C₇₃H₉₀N₁₃O₂₀S₂]⁺: m/z 1532.5861 (FIG. 12).

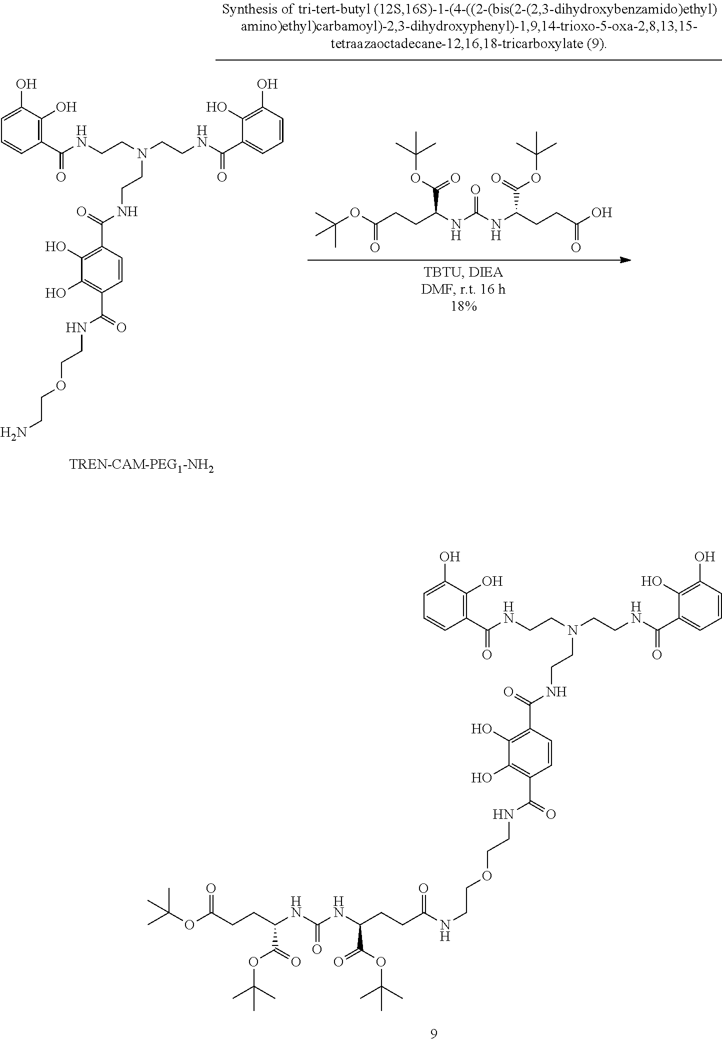 [SMILES: NCCOCCNC(=O)c1ccc(C(=O)NCCN(CCNC(=O)c2cccc(O)c2O)CCNC(=O)c2cccc(O)c2O)c(O)c1O ; CC(C)(C)OC(=O)CC[C@H](NC(=O)N[C@@H](CCC(=O)O)C(=O)OC(C)(C)C)C(=O)OC(C)(C)C ; "TBTU, DIEADMF, r.t. 16 h18%" ; CC(C)(C)OC(=O)CC[C@H](NC(=O)N[C@@H](CCC(=O)NCCOCCNC(=O)c1ccc(C(=O)NCCN(CCNC(=O)c2cccc(O)c2O)CCNC(=O)c2cccc(O)c2O)c(O)c1O)C(=O)OC(C)(C)C)C(=O)OC(C)(C)C]

N¹-(2-(2-aminoethoxy)ethyl)-N⁴-(2-(bis(2-(2,3-dihydroxybenzamido)ethyl)amino)ethyl)-2,3-dihydroxyterephthalamide (0.040 g, 58.4 μmol), (S)-5-(tert-butoxy)-4-(3-((S)-1,5-di-tert-butoxy-1,5-dioxopentan-2-yl)ureido)-5-oxopentanoic acid (0.031 g, 64.3 μmol), TBTU (0.023 g, 70.1 μmol), and DIEA (0.012 mL, 70.1 μmol) were added to a 20 mL vial followed by dry DMF (3 mL). The solution was allowed to stir sealed under Ar overnight. The reaction mixture was concentrated under rotary evaporation at 50° C. The resulting residue was dissolved in MeCN:H₂O (1:1, 800 μL) and the product was isolated via preparatory HPLC (5 μm C18 column, 10% MeCN(aq)-100% MeCN gradient over 1 h with a constant TFA concentration of 0.1%) to yield a white solid (12 mg, 99.7% pure, 18%). ESI-MS: Measured: m/z 1155.5457. Calcd for [C₅₅H₈₀N₈O₁₉]⁺: m/z 1155.5432.

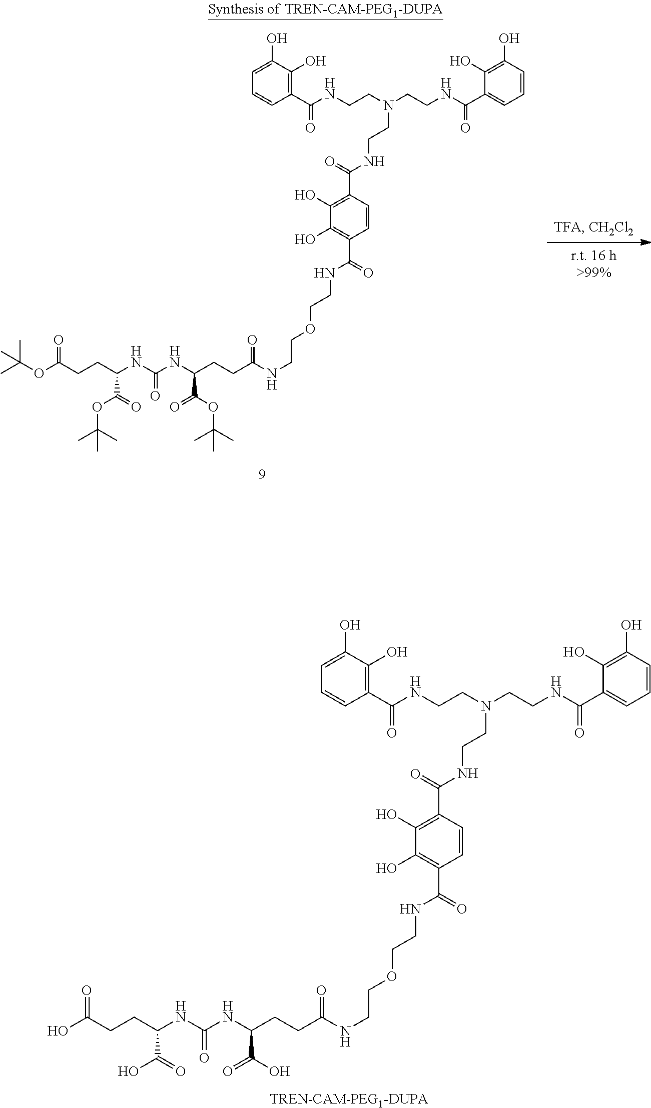

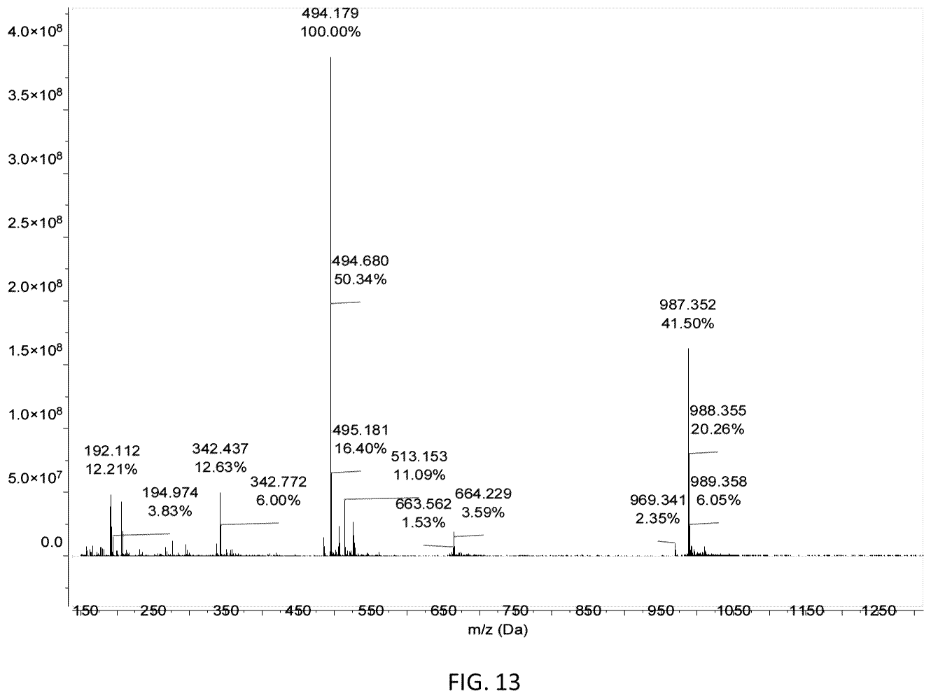

tri-tert-butyl (12S,16S)-1-(4-((2-(bis(2-(2,3-dihydroxybenzamido)ethyl)amino)ethyl) carbamoyl)-2,3-dihydroxyphenyl)-1,9,14-trioxo-5-oxa-2,8,13,15-tetraazaoctadecane-12,16,18-tricarboxylate (10.9 mg, 9.4 μmol) was dissolved in a 2:1 (TFA:dry CH₂Cl₂) mixture (180 μL) and allowed to stir sealed under Ar overnight. The reaction mixture was then concentrated under rotary evaporation. Water (1 mL) was added, the mixture was frozen in liquid N₂ and lyophilized to yield a white solid (TREN-CAM-PEG₁-DUPA, 10.2 mg, 98.9% pure, >99%). ESI-MS: Measured: m/z 494.1823. Calcd for [C₄₃H₅₆N₈O₁₉]²⁺: m/z 494.1826 (FIG. 13).

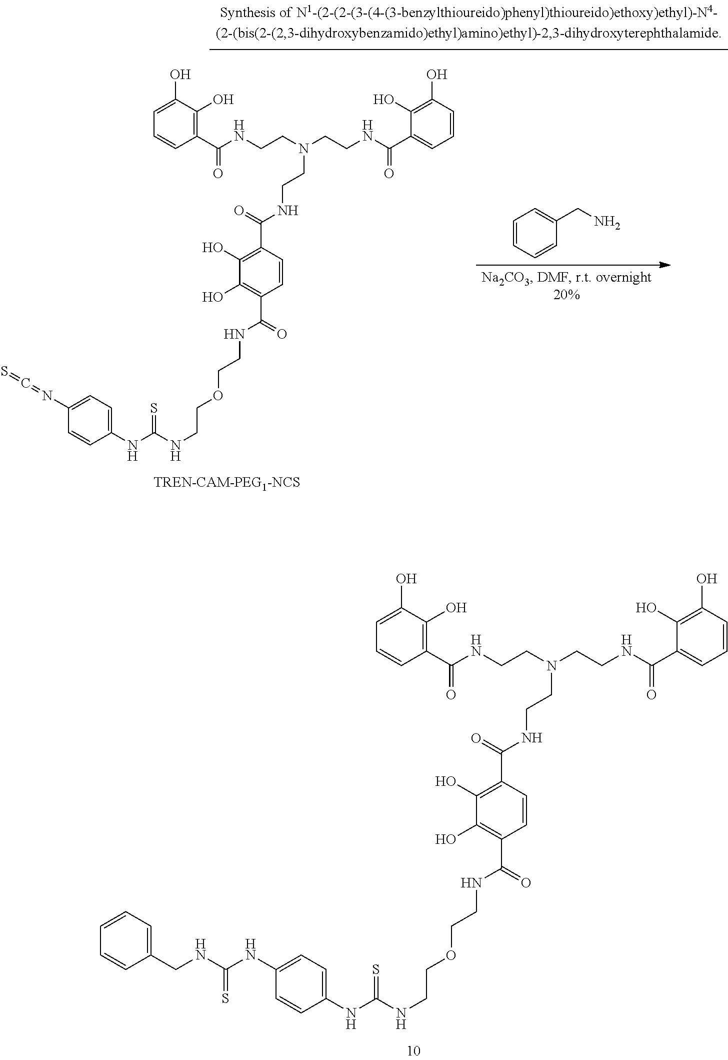 [SMILES: O=C(NCCN(CCNC(=O)c1cccc(O)c1O)CCNC(=O)c1ccc(C(=O)NCCOCCNC(=S)Nc2ccc(N=C=S)cc2)c(O)c1O)c1cccc(O)c1O ; NCc1ccccc1 ; "Na2CO3, DMF, r.t. overnight20%" ; O=C(NCCN(CCNC(=O)c1cccc(O)c1O)CCNC(=O)c1ccc(C(=O)NCCOCCNC(=S)Nc2ccc(NC(=S)NCc3ccccc3)cc2)c(O)c1O)c1cccc(O)c1O]

In a 20 mL vial, TREN-CAM-PEG₁-NCS (9 mg, 10.3 μmol) was dissolved in dry DMF (1.5 mL) followed by addition of benzylamine (1.1 μL, 10.2 μmol) and Na₂CO₃ (10.8 mg, 102 μmol). The reaction mixture was allowed to stir at r.t. sealed under Ar overnight. Upon completion, the reaction mixture was concentrated to dryness under rotary evaporation at 50° C. The resulting residue was dissolved in MeCN:H₂O (1:1, 600 μL) and the product was isolated via preparatory HPLC (5 μm C18 column, 10% MeCN(aq)-100% MeCN gradient over 1 h with a constant TFA concentration of 0.1%) to yield a white solid (2 mg, 96.5% pure, 20%). ESI-MS: Measured: m/z 492.6728. Calcd for [C₄₇H₅₄N₉O₁₁S₂]²⁺: m/z 492.6726.

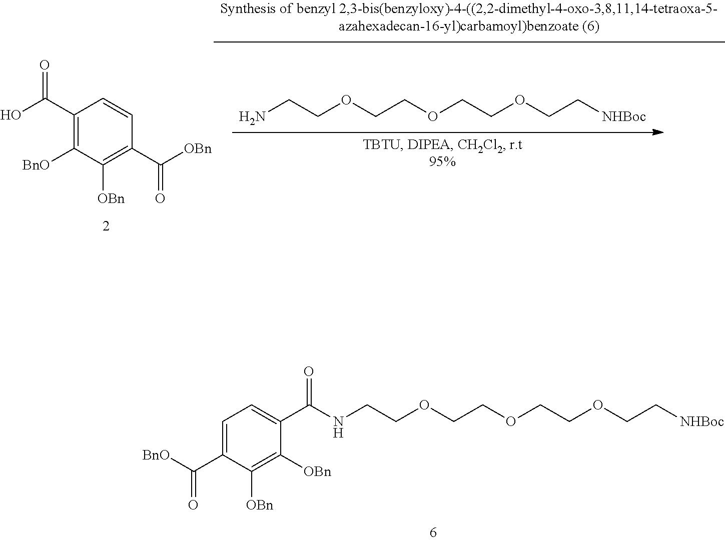

In a 250 mL round-bottom flask, 2,3-bis(benzyloxy)-4-((benzyloxy)carbonyl)benzoic acid (524 mg, 1.12 mmol) was dissolved in dry CH₂Cl₂ followed by addition of DIEA (0.23 mL, 1.34 mmol). Tert-butyl (2-(2-(2-(2-aminoethoxy)ethoxy)ethoxy)ethyl)carbamate (359 mg, 1.23 mmol) was then dissolved in dry CH₂Cl₂ (10 mL) and added to the previous solution followed by addition of TBTU (394 mg, 1.29 mmol). The mixture was allowed to stir at r.t. under N₂ overnight. Upon completion, the reaction mixture was concentrated under rotary evaporation and the product was isolated using an automated column (40 g prepacked silica, 0%-10% MeOH in CH₂Cl₂ gradient) (630 mg, 76%). ¹H NMR (400 MHz, CDCl₃) δ 8.12 (s, 1H), 7.88 (d, J=8.3 Hz, 1H), 7.63 (d, J=8.3 Hz, 1H), 7.43-7.30 (m, 15H), 5.32 (s, 2H), 5.12 (s, 2H), 5.09 (s, 2H), 4.98 (s, 1H), 3.59-3.50 (m, 12H), 3.47 (t, J=5.2 Hz, 2H), 3.26 (q, J=5.5 Hz, 2H), 1.42 (s, 9H).

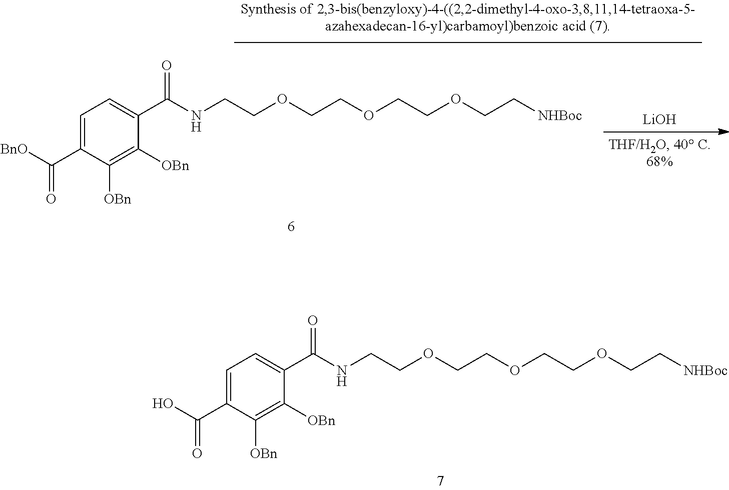 [SMILES: CC(C)(C)OC(=O)NCCOCCOCCOCCNC(=O)c1ccc(C(=O)OCc2ccccc2)c(OCc2ccccc2)c1OCc1ccccc1 ; "LiOH" ; "THF/H2O, 40° C.68%" ; CC(C)(C)OC(=O)NCCOCCOCCOCCNC(=O)c1ccc(C(=O)O)c(OCc2ccccc2)c1OCc1ccccc1]

In a 250 mL round-bottom flask, benzyl 2,3-bis(benzyloxy)-4-((2,2-dimethyl-4-oxo-3,8,11,14-tetraoxa-5-azahexadecan-16-yl)carbamoyl)benzoate (6) (500 mg, 0.673 mmol) was dissolved in THF (20 mL). Then, LiOH (161 mg, 6.73 mmol) was dissolved in H₂O (5 mL) and added to the THF solution. The reaction mixture was allowed to stir at 60° C. overnight. Upon completion, the reaction mixture was cooled to r.t., neutralized with HCl (1 M), and the product was extracted into CH₂Cl₂ (3×30 mL). The organic layers were dried over MgSO₄, filtered, and concentrated under rotary evaporation. The product was isolated using an automated column (24 g Gold prepacked silica, 0%-5% MeOH in CH₂Cl₂ gradient) (300 mg, 68%). ¹H NMR (400 MHz, DMSO-d₆) δ 13.24 (s, 1H), 8.41 (t, J=5.6 Hz, 1H), 7.49 (d, J=8.1 Hz, 1H), 7.46-7.41 (m, 2H), 7.40-7.31 (m, 9H), 6.75 (t, J=5.4 Hz, 1H), 5.05 (d, J=2.3 Hz, 4H), 3.46 (d, J=3.3 Hz, 9H), 3.42-3.30 (m, 6H), 3.03 (q, J=6.0 Hz, 2H), 1.36 (s, 9H).

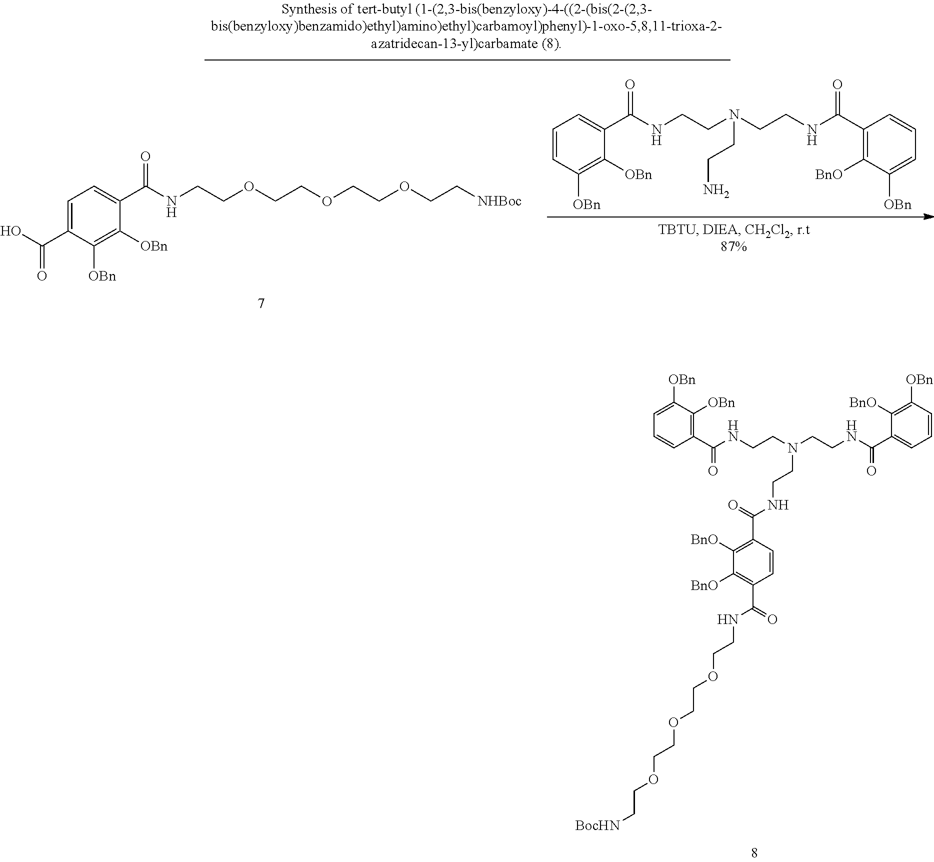 [SMILES: CC(C)(C)OC(=O)NCCOCCOCCOCCNC(=O)c1ccc(C(=O)O)c(OCc2ccccc2)c1OCc1ccccc1 ; NCCN(CCNC(=O)c1cccc(OCc2ccccc2)c1OCc1ccccc1)CCNC(=O)c1cccc(OCc2ccccc2)c1OCc1ccccc1 ; "TBTU, DIEA, CH2Cl2, r.t87%" ; CC(C)(C)OC(=O)NCCOCCOCCOCCNC(=O)c1ccc(C(=O)NCCN(CCNC(=O)c2cccc(OCc3ccccc3)c2OCc2ccccc2)CCNC(=O)c2cccc(OCc3ccccc3)c2OCc2ccccc2)c(OCc2ccccc2)c1OCc1ccccc1]

In a 250 mL round-bottom flask, N,N′-(((2-aminoethyl)azanediyl)bis(ethane-2,1-diyl))bis(2,3-bis(benzyloxy)benzamide) (216 mg, 0.289 mmol) was dissolved in dry CH₂Cl₂ (30 mL), followed by addition of DIEA (60 μL, 0.346 mmol). Then, 2,3-bis(benzyloxy)-4-((2,2-dimethyl-4-oxo-3,8,11,14-tetraoxa-5-azahexadecan-16-yl)carbamoyl)benzoic acid (7) (200 mg, 0.306 mmol) was dissolved in dry CH₂Cl₂ (20 mL) and added to the previous solution followed by addition of TBTU (98 mg, 0.346 mmol). The mixture was allowed to stir at r.t. under N₂ overnight. Upon completion, the reaction mixture was concentrated under rotary evaporation and the product was isolated using an automated column (40 g Gold prepacked silica, 0%-10% MeOH in CH₂Cl₂) (340 mg, 87%). ¹H NMR (400 MHz, DMSO-d₆) δ 8.35 (t, J=5.6 Hz, 1H), 8.20 (t, J=5.6 Hz, 1H), 8.13 (t, J=5.6 Hz, 2H), 7.49 (d, J=6.8 Hz, 4H), 7.42-7.22 (m, 30H), 7.14-7.05 (m, 4H), 6.75 (t, J=5.8 Hz, 1H), 5.18 (s, 4H), 5.03 (d, J=3.2 Hz, 4H), 5.00 (s, 4H), 3.50-3.37 (m, 12H), 3.35 (s, 1H), 3.32 (s, 1H), 3.23 (q, J=6.4 Hz, 6H), 3.04 (q, J=6.1 Hz, 2H), 1.36 (s, 9H).

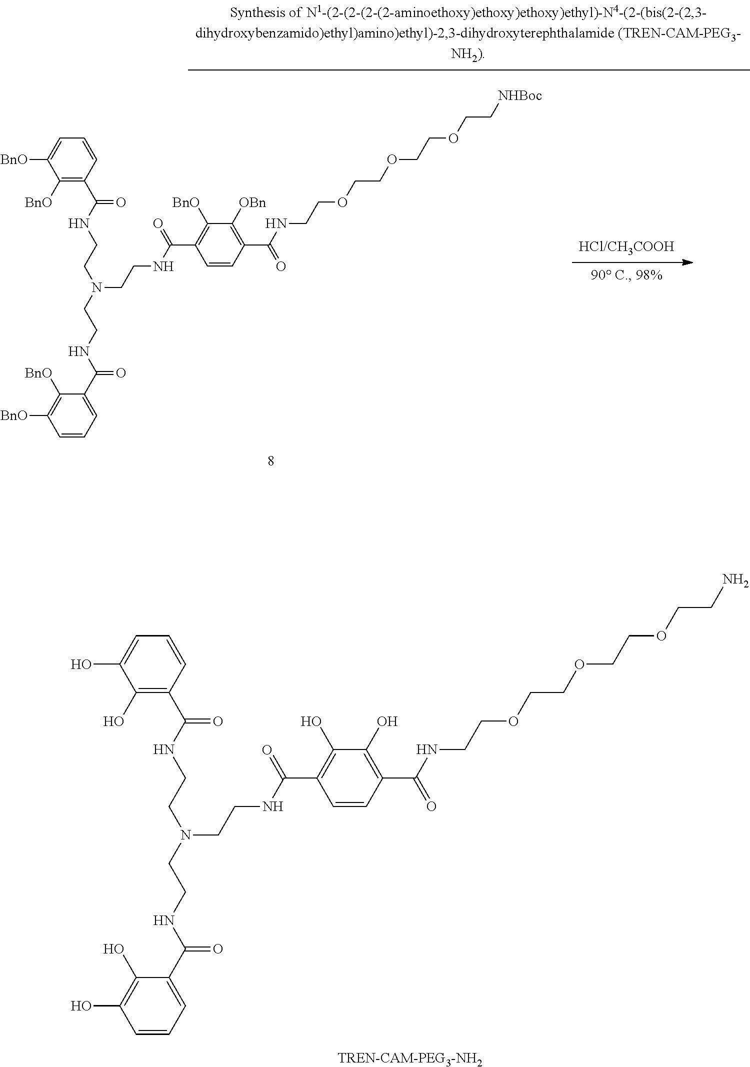 [SMILES: CC(C)(C)OC(=O)NCCOCCOCCOCCNC(=O)c1ccc(C(=O)NCCN(CCNC(=O)c2cccc(OCc3ccccc3)c2OCc2ccccc2)CCNC(=O)c2cccc(OCc3ccccc3)c2OCc2ccccc2)c(OCc2ccccc2)c1OCc1ccccc1 ; "90° C., 98%" ; "HCl/CH3COOH" ; NCCOCCOCCOCCNC(=O)c1ccc(C(=O)NCCN(CCNC(=O)c2cccc(O)c2O)CCNC(=O)c2cccc(O)c2O)c(O)c1O]

In a 50 mL round-bottom flask, tert-butyl (1-(2,3-bis(benzyloxy)-4-((2-(bis(2-(2,3-bis(benzyloxy)benzamido)ethyl)amino)ethyl)carbamoyl)phenyl)-1-oxo-5,8,11-trioxa-2-azatridecan-13-yl) carbamate (340 mg, 0.24 mmol) was dissolved in glacial CH₃COOH (10 mL). Concentrated HCl (aq, 1 mL) was then added, and the reaction mixture was allowed to stir at 90° C. The reaction was monitored by HPLC and had gone to completion after 8 h. Upon completion, the reaction mixture was concentrated under rotary evaporation and purified using preparatory HPLC to isolate the title compound (TREN-CAM-PEG₃-NH₂, 250 mg, 47%). ¹H NMR (400 MHz, DMSO-d₆) δ 12.78 (s, 1H), 12.17 (s, 1H), 12.08 (s, 1H), 9.82 (s, 1H), 9.34 (s, 2H), 9.15-8.86 (m, 4H), 7.78 (s, 2H), 7.38 (d, J=8.8 Hz, 1H), 7.30 (d, J=8.8 Hz, 1H), 7.23 (dd, J=8.3, 1.6 Hz, 2H), 6.93 (d, J=7.9 Hz, 2H), 6.69 (t, J=8.0 Hz, 2H), 3.71 (s, 6H), 3.56 (m, 14H), 3.47 (q, J=5.9 Hz, 6H), 2.97 (q, J=5.6 Hz, 2H).

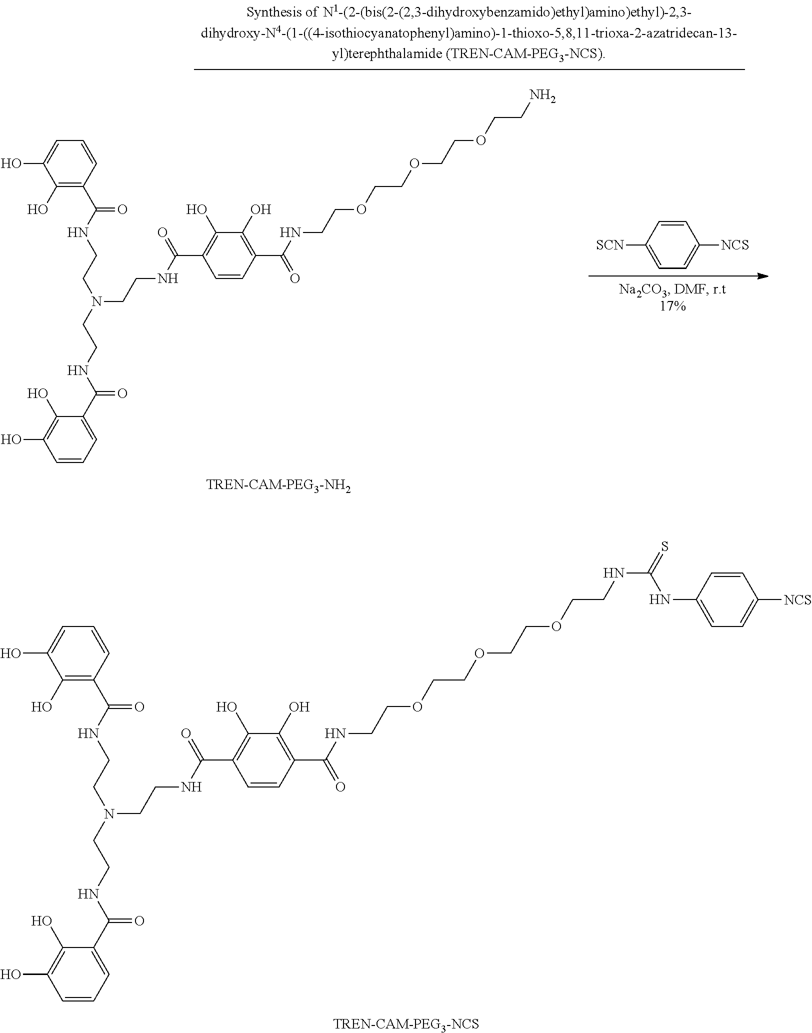

In a 20 mL vial, N¹-(2-(2-(2-(2-aminoethoxy)ethoxy)ethoxy)ethyl)-N⁴-(2-(bis(2-(2,3-dihydroxybenzamido)ethyl)amino)ethyl)-2,3-dihydroxyterephthalamide (TREN-CAM-PEG₃-NH₂) (60 mg, 77.6 μmol) was dissolved in dry DMF (2 mL) followed by addition of Na₂CO₃ (149 mg, 776 μmol). The suspension was allowed to stir at r.t. under Ar for 30 min. Afterwards, p-phenylene diisothiocyanate (82.3 mg, 776 μmol) was added and the mixture was allowed to stir at r.t. sealed under Ar overnight. Upon completion, the reaction mixture was centrifuged, and the supernatant was collected. The residual solid was resuspended in DMF (2 mL). The mixture was centrifuged again, and the supernatants were combined. Diethyl ether (60 mL) was added to the combined supernatant and this mixture was placed in the freezer overnight. Afterward, the solution was centrifuged, the supernatant was decanted, and the remaining solid was dried under reduced pressure. The resulting residue was dissolved in MeCN:H₂O (1:1, 700 μL) and the product was isolated using preparatory HPLC (5 μm C18 column, 10% MeCN(aq)-100% MeCN gradient over 40 min with a constant TFA concentration of 0.1%) to yield a white solid (TREN-CAM-PEG₃-NCS, 12 mg, 17%). ¹H NMR (400 MHz, DMSO-d₆) δ 12.83 (s, 1H), 12.06 (d, J=14.2 Hz, 2H), 9.82 (s, 1H), 9.65 (s, 1H), 9.34 (s, 1H), 8.98 (m, 3H), 7.92 (s, 1H), 7.57 (d, J=8.9 Hz, 2H), 7.37 (dd, J=9.3, 2.5 Hz, 3H), 7.29 (d, J=8.8 Hz, 1H), 7.22 (m, 2H), 6.93 (s, 2H), 6.69 (d, J=7.8 Hz, 2H), 3.65-3.75 (m, 8H), 3.51-3.58 (m, 13H), 3.49-3.44 (m, 8H).

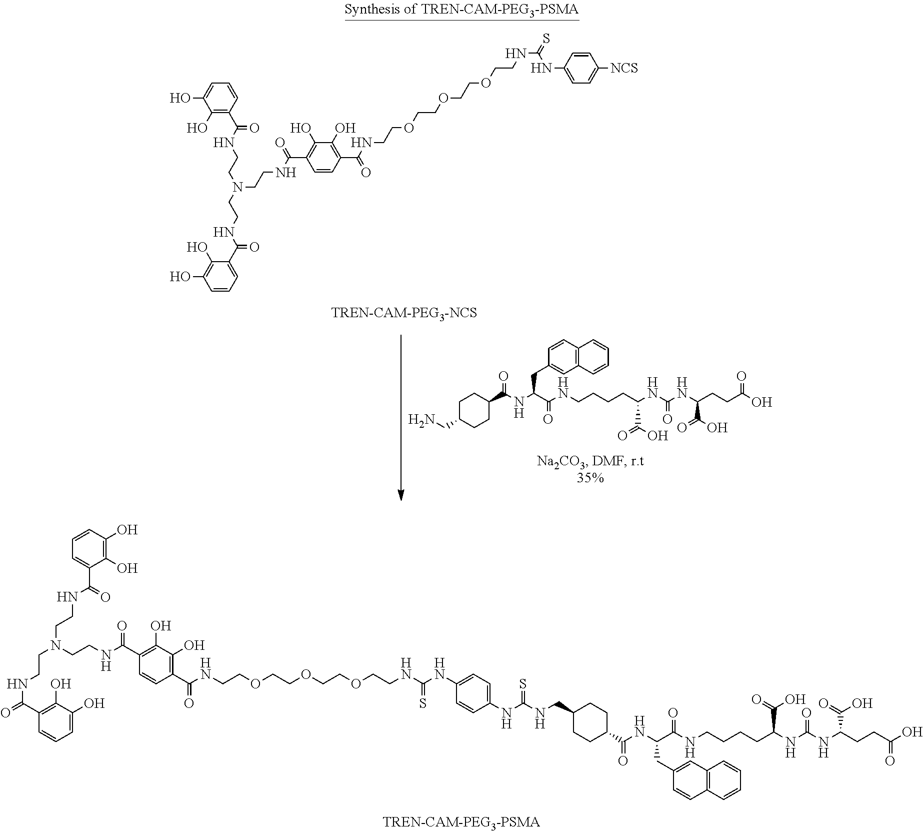

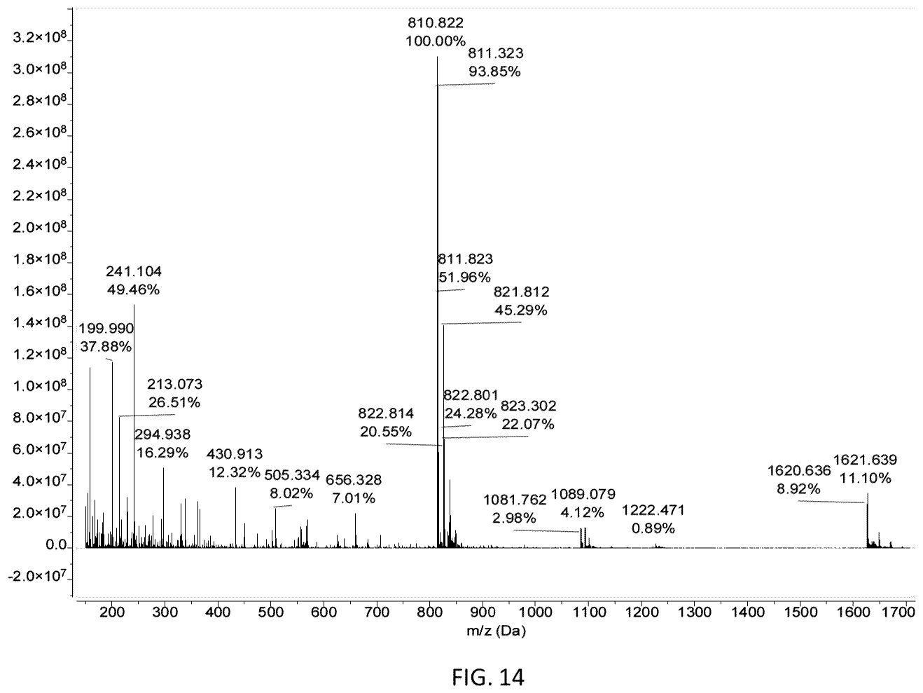

In a 20 mL vial, N1-(2-(bis(2-(2,3-dihydroxybenzamido)ethyl)amino)ethyl)-2,3-dihydroxy-N4-(1-((4-isothiocyanatophenyl)amino)-1-thioxo-5,8,11-trioxa-2-azatridecan-13-yl) terephthalamide (12 mg, 12.43 μmol) was dissolved in 1 mL of anhydrous DMF. Then, the solution was basified with Na₂CO₃ (14 mg, 124.3 μmol, 10 equiv). The suspension was stirred vigorously for 10 min at RT before addition of vipivotide tetraxetan ligand-linker conjugate (PSMA-617 ligand-linker conjugate, MedChemExpress, 8.15 mg, 12.4 μmol, 1 eq). The resulting mixture was stirred in the dark overnight. Upon completion, the reaction mixture was separated via centrifugation and the insoluble salts washed with DMF (2×3 mL). The combined supernatant was diluted with diethyl ether (50 mL) and kept at −20° C. for 2 h to induce precipitation. The crude product was isolated via centrifugation and washed with diethyl ether (2×20 mL). The crude material was then purified via preparative HPLC (A: H₂O (0.1% TFA), B: MeCN (0.1% TFA); 0-5 min: 90% A, 10% B, 5-45 min: 90% A to 100% B, 45-55 min: 100% B, 14 mL/min). Appropriate product fractions were pooled and lyophilized to give the title compound as a white solid (6.9 mg, 35%). ESI-MS: Measured: m/z 1620.6361. Calcd for [C₇₇H₉₈N₁₃O₂₂S₂]⁺: m/z 1620.6386 (FIG. 14).

The word “illustrative” is used herein to mean serving as an example, instance, or illustration. Any aspect or design described herein as “illustrative” is not necessarily to be construed as preferred or advantageous over other aspects or designs.

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

If not already included, all numeric values of parameters in the present disclosure are proceeded by the term “about” which means approximately. This encompasses those variations inherent to the measurement of the relevant parameter as understood by those of ordinary skill in the art. This also encompasses the exact value of the disclosed numeric value and values that round to the disclosed numeric value.

What is claimed is:

1. An antimony chelate comprising an antimony (Sb(V)) radionuclide bound to a chelating ligand having the chemical structure: 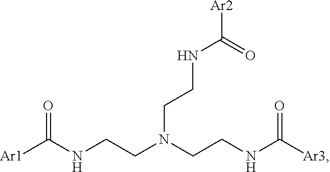
or a pharmaceutically acceptable salt thereof, where Ar1, Ar2, and Ar3 are independently selected from substituted or unsubstituted catechol groups and substituted or unsubstituted hydroxypyridinone (HOPO) groups.

2. The antimony chelate of claim 1, wherein at least one of Ar1, Ar2, and Ar3 comprises a substituted or unsubstituted catechol group.

3. The antimony chelate of claim 1, wherein Ar1, Ar2, and Ar3 each comprises a substituted or unsubstituted catechol group.

4. The antimony chelate of claim 1, wherein at least one of Ar1, Ar2, and Ar3 comprises a substituted or unsubstituted hydroxypyridinone (HOPO) group.

5. The antimony chelate of claim 1, wherein each of Ar1, Ar2, and Ar3 comprises a substituted or unsubstituted hydroxypyridinone (HOPO) group.

6. The antimony chelate of claim 1, wherein at least one of Ar1, Ar2, and Ar3 is a substituted catechol group having the structure: 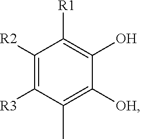
where R1, R2, and R3 are independently selected from H atoms, ring substituents comprising a functional group capable of forming a covalent bond to a biological targeting vector, ring substituents comprising a biological targeting vector, electron withdrawing ring substituents, electron donating ring substituents, and solubilizing ring substituents.

7. The antimony chelate of claim 1, wherein at least one of Ar1, Ar2, and Ar3 has the structure: 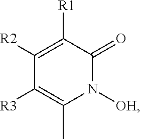
or the structure 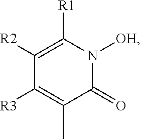
where R1, R2, and R3 are independently selected from H atoms, ring substituents comprising a functional group capable of forming a covalent bond to a biological targeting vector, ring substituents comprising a biological targeting vector, electron withdrawing ring substituents, electron donating ring substituents, and solubilizing ring substituents.

8. The antimony chelate of claim 1, wherein at least one of Ar1, Ar2, and Ar3 is a substituted Me-3,2-HOPO group having the structure: 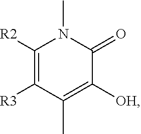
where R2 and R3 are independently selected from H atoms, ring substituents comprising a functional group capable of forming a covalent bond to a biological targeting vector, ring substituents comprising a biological targeting vector, electron withdrawing ring substituents, electron donating ring substituents, and solubilizing ring substituents.

9. The antimony chelate of claim 1, wherein at least one of Ar1, Ar2, and Ar3 has a ring substituent comprising an amine group, a carboxylic acid group, an NCS group, an acyl chloride, an NCO group, an alkyne, an azide, a tetrazine, a maleimide, or an activated ester.

10. The antimony chelate of claim 1, wherein at least one of Ar1, Ar2, and Ar3 is conjugated to a biological targeting vector.

11. The antimony chelate of claim 10, wherein the biological targeting vector is a hormone, a signaling molecule, an antibody, a peptide, an enzyme, a nucleobase-containing moiety, or a lectin.

12. The antimony chelate of claim 1, wherein the antimony (Sb(V)) radionuclide is ¹¹⁹Sb.

13. The antimony chelate of claim 1, wherein the antimony (Sb(V)) radionuclide is ¹¹⁷Sb or ^{118m}Sb.

14. The antimony chelate of claim 9, wherein the ring substituent comprises an amine group attached to Ar1, Ar2, or Ar3 via on organic linker chain comprising an ether group and the organic linker chain is covalently bonded to Ar1, Ar2, or Ar3 via an amide bond.

15. The antimony chelate of claim 14, wherein the organic linker chain comprising an ether group comprising a polyether polyol chain.

16. The antimony chelate of claim 10, wherein the biological targeting vector is attached to Ar1, Ar2, or Ar3 via on organic linker chain comprising an ether group, the organic linker chain is covalently bonded to Ar1, Ar2, or Ar3 via an amide bond, and the organic linker chain is bonded to the biological targeting vector via an amide bond.

17. The antimony chelate of claim 16, wherein the biological targeting vector is Vipivotide tetraxetan or 2-[3-(1,3-dicarboxypropyl)ureido]pentanedioic acid.

18. The antimony chelate of claim 10, wherein the biological targeting vector is attached to Ar1, Ar2, or Ar3 via on organic linker chain comprising an ether group, the organic linker chain is covalently bonded to Ar1, Ar2, or Ar3 via an amide bond, and the organic linker chain is bonded to the biological targeting vector via a thiourea bond.

19. The antimony chelate of claim 18, wherein the biological targeting vector is Vipivotide tetraxetan or 2-[3-(1,3-dicarboxypropyl)ureido]pentanedioic acid.

20. A method of treating a subject in need of treatment, the method comprising administering a therapeutically effective amount of the antimony chelate comprising an antimony (Sb(V)) radionuclide bound to a chelating ligand having the chemical structure: 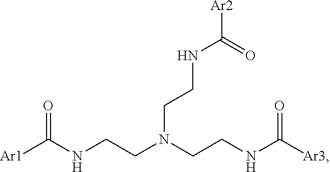
or a pharmaceutically acceptable salt thereof, where Ar1, Ar2, and Ar3 are independently selected from substituted or unsubstituted catechol groups and substituted or unsubstituted hydroxypyridinone (HOPO) groups.
